(19) United States
(12) Patent Application Publication
(10) Pub. No.: US 2024/0029819 A1
(43) Pub. Date: Jan. 25, 2024

(54) AGENTS BINDING MODIFIED ANTIGEN PRESENTED PEPTIDES AND USE OF SAME

(71) Applicant: Yeda Research and Development Co. Ltd., Rehovot (IL)

(72) Inventors: Yifat MERBL, Rehovot (IL); Assaf KACEN, Rehovot (IL); Yishai LEVIN, Rehovot (IL); David MORGENSTERN, Rehovot (IL)

(73) Assignee: Yeda Research and Development Co. Ltd., Rehovot (IL)

(21) Appl. No.: 18/140,095

(22) Filed: Apr. 27, 2023

Related U.S. Application Data
(63) Continuation of application No. PCT/IL2021/051275, filed on Oct. 27, 2021.

(30) Foreign Application Priority Data
Oct. 29, 2020 (IL) 278394

(51) Int. Cl. G16B 15/20; G16B 15/30; G16B 40/20

(52) U.S. Cl. CPC G16B 15/20; G16B 15/30; G16B 40/20

(57) ABSTRACT

Agents binding modified antigen dependent peptides and use of same are provided. Accordingly, there is provided an agent capable of specifically binding an MHC presented peptide comprising a post translational modification (PTM), wherein the agent does not bind a peptide having the same amino acid sequence as said peptide but does not comprise said modification. Also provided are polynucleotides encoding the agent, cells expressing same and methods of use thereof. Also provided is a computer implemented method for generating a dataset of PTM on MHC bound peptides.

### RELATED APPLICATIONS

This application is a Continuation (CON) of PCT Patent Application No. PCT/IL2021/051275 filed on Oct. 27, 2021, which claims the benefit of priority of Israel Patent Application No. 278394 filed on Oct. 29, 2020. The contents of the above applications are all incorporated by reference as if fully set forth herein in their entirety.

### SEQUENCE LISTING STATEMENT

The XML file, entitled 95815 Sequence Listing.xml, created on Apr. 27, 2023, comprising 53,760 bytes, submitted concurrently with the filing of this application is incorporated herein by reference.

### FIELD AND BACKGROUND OF THE INVENTION

The present invention, in some embodiments thereof, relates to agents binding modified antigen dependent peptides and use of same.

The major histocompatibility complex (MHC) molecule serve as a shuttle to transport and display peptide antigens on the surface of cells as an indication to the immune system of the health state of the cells. The species-specific MHC homologues in humans are termed human leukocyte antigens (HLA). MHC bound peptides (i.e., peptides bound to and presented by MHC molecules) originate from proteolysis of most of the proteins expressed in the cells. Therefore, unique sets of peptides are displayed by each of the different MHC haplotypes according to the protein expression and degradation schemes of the cells and according to the peptide binding motifs of the MHC molecules [reviewed e.g. in Neefjes et al. (2011) Nat Rev Immunol 11(12):823-36]. Therefore, thousands of different peptides are presented by the MHC molecules and each of the peptides is presented in different copy number per cell [de Verteuil et al. (2012) Autoimmun Rev. 11(9):627-35].

Targeting tumor antigens that are presented by MHC molecules holds great promise for cancer T cell therapies and immunotherapies. Typically, preferred tumor specific antigens are those present uniquely in tumor cells but are completely absent in non-cancerous tissues and therefore pose minimal risk of inducing autoimmune reactions. Less optimal, but more abundant, are peptides that are expressed at low levels in normal tissues but are over-expressed in tumors, preferably those involved with transformation or cancer progression [Rammensec and Singh-Jasuja (2013) Expert Rev Vaccines 12(10): 1211-1217].

In recent years, post-translational modifications (PTMs), such as phosphorylations, citrullinations or glycosylations¹⁰⁻¹⁶, have also been reported to modulate antigen presentation and recognition. These may be affected by changes in signaling pathways or in the activity of modifying enzymes in the cancerous state. However, due to the difficulties in detecting them, whether and to what extent such PTM alterations expand the landscape of antigenic targets in cancer, remained under-explored.

Current technologies for target antigen discovery rely mostly on genomic or transcriptomic data²⁷ combined with computational prediction tools for HLA binding²⁸⁻³⁰. Such data lacks information on the state of modification of the peptides. Mass Spectrometry (MS) based immunopeptidomics allows for the identification of MHC-bound peptides by immunoprecipitation of the MHC-peptide complex from the surface of cells and eluting the bound peptides. Detection of PTMs on such peptides generally still requires biochemical enrichment of the modification of interest^{15,31-34}. For example, phosphopeptides were identified through dedicated protocols¹¹, or specialized prediction software³⁵. However, even if one captures modified peptides with MS, they cannot be identified with the standard algorithms, which search against the canonical amino acid sequence. Adding potential modifications and non-canonical sequences to the theoretical search space exponentially increases the number of peptide possibilities, making search times impractical. Therefore, the vast majority of PTMs, and combination thereof, have not been examined to date.

### SUMMARY OF THE INVENTION

According to an aspect of some embodiments of the present invention there is provided an agent capable of specifically binding an MHC presented peptide comprising a post translational modification (PTM), wherein the peptide amino acid sequence is selected from the group consisting of SEQ ID NO: 1-10746, 10817, 10819, 10820, 10823, 10824, 10826 and 10827 having the corresponding modification according to Table 3, and wherein the agent does not bind a peptide having the same amino acid sequence as the peptide but does not comprise the modification.

According to an aspect of some embodiments of the present invention there is provided an agent capable of binding an MHC presented peptide, wherein the peptide comprises a ubiquitin or a ubiquitin-like (UBL) modifier tail, and wherein the agent does not bind a peptide having the same amino acid sequence as the peptide but does not comprise the tail.

According to some embodiments of the invention, the peptide amino acid sequence is selected from the group of sequences listed in Table 5.

According to an aspect of some embodiments of the present invention there is provided an agent capable of specifically binding an MHC presented peptide selected from the group consisting of SEQ ID NO: 10747-10816 and 10822.

According to some embodiments of the invention, the agent binds the peptide in an MHC-restricted manner.

According to some embodiments of the invention, the MHC is MHC class I.

According to some embodiments of the invention, the MHC is HLA class I.

According to some embodiments of the invention, the HLA class I comprises a haplotype selected from the group consisting of HLA-A0201, HLA-B5401, HLA-B5101, HLA-A6802. HLA-B4402, HLA-B4403 and HLA-A3101.

According to some embodiments of the invention, the agent is an antibody.

According to some embodiments of the invention, the agent is a T cell receptor (TCR) or a chimeric antigen receptor (CAR).

According to some embodiments of the invention, the agent comprises a therapeutic moiety.

According to some embodiments of the invention, the therapeutic moiety is selected from the group consisting of a toxin, a drug, a chemical, a protein and a radioisotope.

According to some embodiments of the invention, the therapeutic moiety is capable of eliciting an immune response to a cell presenting the peptide.

According to an aspect of some embodiments of the present invention there is provided a polynucleotide encoding the agent.

According to an aspect of some embodiments of the present invention there is provided a cell expressing the agent.

According to some embodiments of the invention, the cell is an immune cell.

According to some embodiments of the invention, the immune cell is a T cell.

According to an aspect of some embodiments of the present invention there is provided a method of eliciting an immune response in a subject in need thereof, the method comprising administering to the subject an effective amount of the agent or the cell, thereby eliciting an immune response in the subject.

According to an aspect of some embodiments of the present invention there is provided a method of treating cancer in a subject in need thereof, the method comprising administering to the subject a therapeutically effective amount of the agent or the cell, thereby treating the cancer in the subject.

According to an aspect of some embodiments of the present invention there is provided the agent or the cell, for use in treating cancer in a subject in need thereof.

According to an aspect of some embodiments of the present invention there is provided a method of eliciting an immune response in a subject in need thereof, the method comprising administering to the subject an effective amount of a peptide comprising an amino acid sequence selected from the group consisting of SEQ ID NO: 1-10746, 10817, 10819, 10820, 10823, 10824, 10826 and 10827 having the corresponding modification according to Table 3, thereby eliciting an immune response to a cell presenting the amino acid sequence having the corresponding modification in the subject.

According to an aspect of some embodiments of the present invention there is provided a method of eliciting an immune response in a subject in need thereof, the method comprising administering to the subject an effective amount of a peptide comprising an amino acid sequence having a ubiquitin or a ubiquitin-like (UBL) modifier tail, thereby eliciting an immune response to a cell presenting the amino acid sequence having the ubiquitin or the UBL modifier tail in the subject.

According to an aspect of some embodiments of the present invention there is provided a method of eliciting an immune response in a subject in need thereof, the method comprising administering to the subject an effective amount of a peptide comprising an amino acid sequence selected from the group consisting of SEQ ID NO: 10747-10816 and 10822, thereby eliciting an immune response to a cell presenting the amino acid sequence in the subject.

According to an aspect of some embodiments of the present invention there is provided a method of treating cancer in a subject in need thereof, the method comprising administering to the subject a therapeutically effective amount of a peptide comprising an amino acid sequence selected from the group consisting of SEQ ID NO: 1-10746, 10817, 10819, 10820, 10823, 10824, 10826 and 10827 having the corresponding modification according to Table 3, thereby treating the cancer in the subject.

According to an aspect of some embodiments of the present invention there is provided a method of treating cancer in a subject in need thereof, the method comprising administering to the subject a therapeutically effective amount of a peptide comprising an amino acid sequence having a ubiquitin or a ubiquitin-like (UBL) modifier tail, thereby treating the cancer in the subject.

According to an aspect of some embodiments of the present invention there is provided a method of treating cancer in a subject in need thereof, the method comprising administering to the subject a therapeutically effective amount of a peptide comprising an amino acid sequence selected from the group consisting of SEQ ID NO: 10747-10816 and 10822, thereby treating the cancer in the subject.

According to an aspect of some embodiments of the present invention there is provided a peptide comprising an amino acid sequence selected from the group consisting of SEQ ID NO: 1-10746, 10817, 10819, 10820, 10823, 10824, 10826 and 10827 having the corresponding modification according to Table 3, for use in treating cancer in a subject in need thereof.

According to an aspect of some embodiments of the present invention there is provided a peptide comprising an amino acid sequence having a ubiquitin or a ubiquitin-like (UBL) modifier tail, for use in treating cancer in a subject in need thereof.

According to an aspect of some embodiments of the present invention there is provided a peptide comprising an amino acid sequence selected from the group consisting of SEQ ID NO: 10747-10816 and 10822, for use in treating cancer in a subject in need thereof.

According to some embodiments of the invention, the amino acid sequence is selected from the group of sequences listed in Table 5.

According to some embodiments of the invention, the peptide is capable of eliciting an immune response to a cell presenting the amino acid sequence having the corresponding modification or the ubiquitin or UBL modifier tail.

According to some embodiments of the invention, the peptide is capable of eliciting an immune response to a cell presenting the amino acid sequence.

According to some embodiments of the invention, the peptide is capable of being presented by a MHC molecule.

According to some embodiments of the invention, the peptide amino acid sequence consists of the amino acid sequence.

According to some embodiments of the invention, the peptide is administered in a composition comprising an adjuvant.

According to some embodiments of the invention, the peptide is administered in a composition comprising an antigen presenting cell for presenting the peptide.

According to some embodiments of the invention, the antigen presenting cell is a dendritic cell.

According to an aspect of some embodiments of the present invention there is provided a method of detecting a cancer cell in a subject, the method comprising determining in a biological sample of the subject a cell surface level of a peptide having an amino acid sequence selected from the group consisting of SEQ ID NO: 1-10746, 10817, 10819, 10820, 10823, 10824, 10826 and 10827 and the corresponding modification according to Table 3, wherein a level of the peptide above a predetermined threshold and/or increased level relative to a reference biological sample of a healthy subject is indicative of presence of cancer cell in the subject, thereby detecting cancer cell in the subject.

According to an aspect of some embodiments of the present invention there is provided a method of detecting a cancer cell in a subject, the method comprising determining in a biological sample of the subject a cell surface level of a peptide selected from the group consisting of SEQ ID NO: 10747-10816 and 10822, wherein a level of the peptide above a predetermined threshold and/or increased level relative to a reference biological sample of a healthy subject is indicative of presence of cancer cell in the subject, thereby detecting cancer cell in the subject.

According to some embodiments of the invention, the cancer is selected from the group consisting of glioblastoma, B cell leukemia, meningioma, melanoma, colon cancer and breast cancer.

According to some embodiments of the invention, when the peptide amino acid sequence is selected from the group consisting of SEQ ID NO: 1-209 and 10819; the cancer is B cell leukemia, when the peptide amino acid sequence is selected from the group consisting of SEQ ID NO: 210-943; the cancer is breast cancer, when the peptide amino acid sequence is selected from the group consisting of SEQ ID NO: 944-1117 and 10820; the cancer is colon cancer, when the peptide amino acid sequence is selected from the group consisting of SEQ ID NO: 1118-1691 and 10817: the cancer is glioblastoma, when the peptide amino acid sequence is selected from the group consisting of SEQ ID NO: 1962-8276; the cancer is melanoma cancer and/or when the peptide amino acid sequence is selected from the group consisting of SEQ ID NO: 8277-8897; the cancer is meningioma.

According to an aspect of some embodiments of the present invention there is provided a computer implemented method for generating a dataset of post translations modifications (PTM) on major histocompatibility complex (MHC) bound peptides, comprising:
receiving a mass spectrometry (MS) dataset obtained from a sample of cells associated with a target disease for treatment, the MS dataset storing a plurality of spectra data elements outputted by a MS device analyzing MHC bound peptides to generate a plurality of amino acid sequences, each spectra data element for a respective amino acid sequence of the MHC bound peptides;
   receiving a reference sequence dataset storing amino acid sequences of proteins;
   receiving a variable modification dataset storing a plurality of modifications each including a respective amino acid and expected mast shift;
   generating a plurality of combination, each combination including a respective amino acid sequence selected from the reference sequence dataset and at least one modification selected from the variable modification dataset;
   searching using a plurality of processors connected in parallel, wherein each processor searches for a respective spectra element on the plurality of combinations to identify a plurality of best peptide to spectra matches (PSMs), wherein each respective processor assigns a ranking score to respective PSM according to the respective search performed by the respective processor;
   aggregating the plurality of PSMs from the plurality of processors connected in parallel to generate a main PSM list with main ranking score by computing the main ranking score from the ranking score of each respective PSM of each respective search;
   selecting highest ranking PSMs according to respective main ranking scores;
   storing in a modified sequence dataset, a plurality of modified sequences each including the PTM and sequences corresponding to the selected highest ranking PSMs, wherein the modified sequence dataset stores an indication of binding motifs defined by a plurality of identified PTM and corresponding sequence; and
   providing the modified sequence dataset for selecting a certain binding motif having a certain PTM and corresponding amino acid sequence from the modified sequence dataset capable of specifically binding an MHC presented peptide for treatment of the target disease.

According to some embodiments of the invention, the method further comprising:
creating a training dataset by labelling each modified sequence for each respective motif of the modified sequence dataset, each modified sequence including an amino acid sequence. PTM type, and position of the PTM on the amino acid sequence, each label including an indication of one or more of: an MHC type, parent gene, and position of the motif within a full protein length; and
training a machine learning (ML) model using the training dataset, wherein for an input of a certain modified sequence defined by a combination of an amino acid sequence and at least one PTM into the ML model, an indication of whether the certain modified sequence is predicted to fit a binding motif that binds to a cell of the MHC type is obtained as an outcome of the ML model, and
   for an input of an amino acid sequence of a full protein length and PTMs into the ML model, at least one modified sequence predicted to fit a binding motif is obtained as an outcome of the ML model.

According to some embodiments of the invention, at least one of:
the modified sequence dataset stores peptides selected from the group consisting of SEQ ID NO: 1-10746, 10817, 10819, 10820, 10823, 10824, 10826 and 10827,
the target disease comprises cancer, and the certain binding motif is selected for treating the cancer using immunotherapy, and
the MHC comprises HLA I.

According to some embodiments of the invention, searching comprises:
allocating a respective subset of the plurality of combinations to a plurality of processors connected for parallel processing, each respective processors searching the respective spectra element on the respective subset to identify a respective set of PSM,
   merging the respective set of PSM of each respective processor to create a PSM aggregation dataset,
wherein the highest ranking PSMs are selected from the PSM aggregation dataset.

According to some embodiments of the invention, statistical parameters used in a subsequent false discovery rate (FDR) calculation are distorted by a plurality of searches of a same reference dataset over different software instances executed by the plurality of processors, and wherein merging further comprises:
removing duplicated PSM from the PSM aggregation dataset by using unmodified hits combined histogram to evaluate a number of duplicated PSM and identify the duplicated PSM for removal thereof, and
   recalculating an expectation based on a restored score histogram for each PSM.

According to some embodiments of the invention, the method further comprising:
computing a plurality of quality assignment measures, and performing the following using the quality assignment measures:
validating the PTM of each member of the PSM aggregation dataset according to the quality measures;
filtering ambiguous assignments and isobaric decoys of the PSM aggregation dataset according to a filtering threshold;
ranking members of the PSM aggregation dataset; and
selecting the highest ranking PSMs according to the highest ranked member of the PSM aggregation dataset.

According to some embodiments of the invention, the method further comprising:
computing a probability score indicative of match accuracy for each PSM, wherein the highest ranking PSMs are selected according to highest probability.

According to some embodiments of the invention, the method further comprising:
dividing the PSM aggregation dataset into groups including: unmodified, standard search modification types, and other modification types, using a threshold cutoff based on respective abundance in the PSM aggregation dataset;
for each group the PSM are sorted by probability score and a threshold is set for assuring false identification is below the FDR limits.

According to some embodiments of the invention, a difference in probability scores is below a defined percentage of the average probability score, the lower-ranked PSM are obtained and added to the modified sequence dataset.

According to some embodiments of the invention, a certain PSM is identified as the highest ranking PSMs when the certain PSM is identified as having a highest probability score in one respective set of PSM and a lower ranked probability score in another respective set of PSM.

According to some embodiments of the invention, the method further comprising:
extracting the peaks from the PSM;
for each peak, computing a plurality of theoretical fragment ions for an unmodified version of the respective peptide and adjust each theoretical fragment ion according to the modification mass shift, and annotating the respective peak with the theoretical fragment ions.

According to some embodiments of the invention, the plurality of theoretical fragment ions includes a, b, y precursor and diagnostic ions with potential ammonium and water lost in expected peptide charges.

According to some embodiments of the invention, the method further comprising: for each PSM, searching for modification reporter ions, providing a number of b and y ions, and computing a proportion of ion current (PIC),

wherein unassigned peaks with significant intensity indicate a discrepancy between an observed spectrum defined by the respective spectra element of the plurality of PSMs and a matched peptide of the PSM.

According to some embodiments of the invention, the method further comprising:
for each PTM of each PSM, creating a window of potential site positions based on the annotated peaks.

According to some embodiments of the invention, at least one of: (i) including alternative site positions within the window, and (ii) including alternative combinations of modifications with equivalent mass.

According to some embodiments of the invention, for each respective PTM of each identified PSM:
searching for identical masses or combination of masses that match the respective PTM mass shift indicative of mass decoy and/or isobaric masses, and in response to finding the identical masses or combination of masses, removing the ambiguous respective identified PSM corresponding to the respective PTM.

According to some embodiments of the invention, the method further comprising excluding PSM with total peptide mass greater than average mass of a maximum peptide length plus a tolerance value.

According to some embodiments of the invention, the method further comprising, for each respective PSM, searching in a dataset of known PSM of healthy cells and cells with the target disease for a match, and increasing likelihood of the respective PSM being included in the modified sequence dataset when the PSM is found in the dataset of known PSM.

According to an aspect of some embodiments of the present invention there is provided a method for creating a ML model for predicting when a modified sequence binds to MHC, comprising:
creating a training dataset by labelling each modified sequence for each respective motif of the modified sequence dataset, each modified sequence including an amino acid sequence. PTM type, and position of the PTM on the amino acid sequence, the modified sequence dataset created as described, each label including an indication of one or more of: an MHC type, parent gene, and position of the motif within a full protein length; and
   training a machine learning (ML) model using the training dataset,
wherein for an input of a certain modified sequence defined by a combination of an amino acid sequence and at least one PTM into the ML model, an indication of whether the certain modified sequence is predicted to fit a binding motif that binds to a cell of the MHC type is obtained as an outcome of the ML model, and
   for an input of an amino acid sequence of a full protein length and PTMs into the ML model, at least one modified sequence predicted to fit a binding motif is obtained as an outcome of the ML model.

According to an aspect of some embodiments of the present invention there is provided a computer implemented method of predicting a motif on a target HLA complex, comprising
receiving an input of one of: (i) a certain modified sequence defined by an amino acid sequence and a PTM, and (ii) an amino acid sequence of a full protein length and PTMs;
feeding the input into an ML model; and
obtaining as an outcome of the ML model, for the input of (i) an indication of whether the certain modified sequence is predicted to fit a motif that binds to a cell of the MHC type, and for the input of (ii) obtaining at least one motif predicted to be created from the full protein length and PTMs.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H demonstrate that the computation pipeline for global search of PTMs on HLA-bound peptides enriches identifications by 11%. FIG. 1A is a schematic representation demonstrating that the protein Modification Integrated Search Engine (PROMISE) allows for the systematic detection of modifications on HLA peptides. FIG. 1B is a pie chart of peptides identified in the standard and multi-modification search performed on multiple immunopeptidomics datasets. Modified peptides identified only with the PROMISE analysis enriched total peptide identification by 11% (red line) compared to the original search (grey line). Enriched peptides were either matched to previously unassigned spectra (dark red) or improved an existing match with an assignment to a higher scoring peptide (light red). FIG. 1C-D are graphs demonstrating comparison of the amino acid composition of peptides identified in the standard or PROMISE search (FIG. 1C) or the unmodified and modified subsets of peptides in the PROMISE search (FIG. 1D). Circle size and color indicate the log 2 transformed ratio of amino acid abundance between the two subsets. FIG. 1E demonstrates the distribution of the lengths of modified and unmodified peptides. In FIGS. 1F-H the modifications are divided into those that may arise during sample processing (“technical”-shades of orange) and those that reflect the cellular state (“Biological”—blues). Peptides identified in standard search (FIG. 1F) or PROMISE (FIG. 1G) are binned by number and type of modification. When viewed by modification site, 33,481 positions were uniquely identified by PROMISE in the immunopeptidomics datasets analyzed. These sites are then presented in a pie chart divided by modification type, and amino acid modified (FIG. 1H).

FIGS. 2A-G demonstrate PTM driven binding preference highlighted through unbiased search of 29 modifications. FIG. 2A shows all the modified peptides identified with the re-analysis of the Bassani et al¹ dataset by PROMISE (n=12.268 peptides), sorted by the modification type and position in the peptide. Each line represents a distinct peptide in grey with the modification(s) site colored. For the peptides with more than one modification, the leading modification was defined by prioritizing biological modification over a technical one. The modification position can be evenly distributed in the peptide or reveal a distinct location tendency. FIG. 2B demonstrates length distribution of the percentage of peptides (density) at the indicated lengths with acetylation from the protein n-terminus (“nAcetylation”, blue) and length distribution of the other modified peptides (grey). Dotted line indicates mean length. In FIG. 2C-G the modified amino acid position distribution (“Modified”, red) was compared to the distribution of the unmodified amino acid that carries this modification in the analyzed datasets (“background”, grey) or identified in the IEDB² database (“IEDB”, blue). Major differences between those distributions suggest that the modified amino acid has position preferences not solely determined by the properties of the unmodified amino acid. Below each histogram, the fold change between the modified AA and unmodified AA distribution is presented as a heatmap bar (red indicates overrepresentation of the modified AA relative to the unmodified distribution). FIG. 2C demonstrates that the correlation between oxidized methionine position distribution and the un-modified methionine distribution is very high (Pearson 0.96, p value 1.05e-6), and as expected from a technical artifact the distributions are not significantly different (F-test; p value=0.1339). FIG. 2D shows the distribution of serine demonstrating that the phosphorylated form falls predominantly falls in the 4th position and significantly different from the unmodified serine distribution (F-test; p value=1.022e-14). In FIG. 2E the modification distributions are sorted by the correlation between the modified amino acid and the un-modified background. A low correlation means the PTM distribution is distinct from the unmodified background, suggesting a PTM-driven motif. FIG. 2F demonstrates that lysine residues are underrepresented at the second position of the peptide, however the distribution of the dimethylated form is enriched at the second position compared to the background (F-test; p value=2.2e-16). FIG. 2G demonstrates that methylated arginine is enriched in positions 3 to 7 compared to background arginine (F-test; p value=2.643e-13).

FIGS. 3A-G demonstrate the PTM driven HLA motif. In FIG. 3A, a recognition area score was calculated to determine the tendency of a given modification to be located in the MHC anchor position (purple) or center of the peptide (green) for a given HLA haplotype. FIGS. 3B-E demonstrates motif of the reported unmodified epitopes in the IEDB database for the indicated haplotype (top). The canonical modified motif was then compared to the amino acid motif for a given modification (middle). The histogram then represents the modified amino acid frequency in each position (red) compared to the unmodified amino acid background (grey). Each motif/histogram contains positions 1-7 from the N-terminus and the C-terminus and the preceding position (C-1). Overall, 9 mer epitopes are presented naturally with all their positions, positions 7 and C-1 are identical for 8 mer epitopes and peptides longer than 9 are truncated accordingly. FIG. 3B demonstrates Chemical mimics motif: Aspartic acid is favored in the A0101 binding motif at position 3. Because deamidated asparagine is chemically similar to aspartic acid, it has a similar distribution, while unmodified asparagine is not found in position 2. FIG. 3C demonstrates Binding interference: acetylated lysine is under-represented in the C-terminus of haplotype A0301 and altering the peptide to become an unfavorable binder. Figures D-E demonstrates novel motif: methylated glutamine at the peptide C-terminus in haplotype B5401 and oxidized proline at the anchor position 2 of haplotype A0201 create favorable binder peptides, which are different from the known unmodified motif. FIG. 3F-G show Rosetta FlexPepDock structural models of the interactions between the modified peptide (yellow sticks) and the MHC molecule (grey surface cartoon). The modified amino acid (green) creates a more stable interaction with the MHC molecule as compared to the unmodified form. The effect of the modified amino acid is shown in detail in the zoom-in picture. FlexPepDock reweighted score was calculated for the interaction between the MHC and modified or unmodified peptide. More negative score indicates a more stable interaction. FIG. 3D demonstrates the interaction between K(ac)P(ox)SLEQSPAVL (SEQ ID NO: 10817 having the recited modifications) and haplotype HLA-A0201: the proline hydroxyl group at position 2 forms a stabilizing hydrogen bond with MHC receptor residue E-87, while the lysine acetyl group at position 1 forms a hydrogen bond with K-90 (both shown as dashed green lines left and right, respectively). Other hydrogen bonds between peptide and receptor are shown in yellow dashed lines. FIG. 3G demonstrates the interaction between MPTLPPYQ(me) (SEQ ID NO: 10818 having the recited modification) and haplotype HLA-B5401: Methylation reduces the polar character of the glutamine side chain, allowing for stabilizing interaction with the c-terminal anchor pocket. The glutamine methyl group is shown as green sphere, MHC interacting residues shown as gray spheres. The modified peptide shows significant lower predicted affinity (measured as FlexPepDock reweighted score).

FIGS. 4A-F demonstrate that modified HLA-bound peptides create cancer-specific signatures. In FIG. 4A modified peptides from the Bassani et al¹ dataset (n=8700 peptides), were clustered, revealing a cancer-specific signature (left heatmap). For each modified peptide, the signal intensity ratio as compared to the unmodified peptide is presented using the same coordinates as the modified heatmap (right heatmap; grey indicates signal ratio, red indicates only the modified peptide was identified). Each modification type was then clustered as a separate group and a correlation was measured between the modified and unmodified peptide abundance for that group (“corr”, green). The order of modification types is sorted by the correlation value. A list of peptides of interest with their parent protein is shown on the left (SEQ ID NOs: 86, 10819, 10820, 139, 10821, 10822, 2192 having the recited modifications), colored blocks indicate the cell line in which the peptide was detected. In FIG. 4B the percent of immunopeptides identified with each of the indicated modifications was calculated for a cohort of triple-negative breast cancer tumors and adjacent tissue (Temette, N. et al³). The modifications are sorted from the most enriched in the tumor tissue at the top to the most enriched in adjacent tissue at the bottom. A students T-test was used to determine significance of the observed change in percentage: Cysteine cysteinylation is significantly enriched in the tumor (***p=0.00045) while histidine oxidation (*p=0.044), arginine citrullination (*p=0.013), lysine ubiquitination (**p=0.0031) and cysteine carbamidomethylation (**p=0.0078) are significantly enriched in the normal tissue. In FIGS. 4C-D each list of antigens is sorted by the modification of the peptide. For each peptide the cancer annotation is marked (driver, oncogene, tumor suppressor) as documented in CancerMine⁴ if the peptide was reported in IEDB 2 in its unmodified state, and if it is a cancer-testis antigens. For a cohort of patient samples (orange) the color indicates the percentage of the patients the peptide was identified in. For cancer cell lines (blue) the color indicates that the peptide was detected. FIG. 4C shows modified a list of cancer-testis antigens (n=244) and a list of shared antigens (n=400) identified through the modified state. FIG. 4D shows a list of HLA-A0201 bound modified peptides that were not reported in the IEDB database. FIG. 4E shows Rosetta FlexPepDock structural model of the interactions between TLIESK(me)LPV (SEQ ID NO: 10823 having the recited modification, yellow sticks) and the HLA-A0201 molecule (grey surface/cartoon). The methylated lysine (green) is packed against hydrophobic residues of the MHC molecule (gray spheres). The modification created a more stable interaction with the MHC molecule. In FIG. 4F, 6 modified peptides and their matching unmodified form from the list in FIG. 4D were tested for binding affinity through ProImmune in-vitro binding assay (SEQ ID NOs 10824, 10823, 9194, 9827, 10825, 10826 having the recited modifications). TLN(d)SLIYTL (SEQ ID NO: 10824 having the recited modification) was found to bind more strongly in its unmodified form. By contrast. TLIESK(me)LPV (SEQ ID NO: 10823 having the recited modification) and K(me)VMDEVAGI (SEQ ID NO: 9194 having the recited modification) were both found to bind the HLA-A0201 more strongly than the unmodified form. TLE(me)NCLLPD(me) (SEQ ID NO: 10825 having the recited modifications) bound the MHC only in its modified form.

FIG. 5 demonstrates KP(ox)LKVIFV (SEQ ID NO: 10827 having the recited modification) and HLA-A0201 3D interaction. Shown a Rosetta FlexPepDock structural model of the interaction between the modified peptide KP(ox)LKVIFV (SEQ ID NO: 10827 having the recited modification, yellow sticks) and the MHC molecule haplotype HLA-A0201 (grey surface\cartoon). The modified amino acid (green) creates a more stable interaction with the MHC molecule as compared to the unmodified form. The effect of the modified amino acid is shown in detail in the zoom-in picture. The proline hydroxyl group at position 2 forms a stabilizing hydrogen bond with MHC receptor residue E-87 (shown as dashed yellow line, as well as other hydrogen bonds between peptide and receptor). FlexPepDock reweighted score was calculated for the interaction between the MHC and modified or unmodified peptide. A more negative score indicates a more stable interaction.

FIGS. 6A-B shows example of peptides that were detected by analysis of Bassani et al¹ dataset with PROMISE (SEQ ID NOs: 86, 10819, 10820, 139, 10821, 10822, 3069 having the recited modifications). The modified form of the peptides was detected and the unmodified form was not. These peptides were uniquely detected in a specific cancer cell line. SPAG9 and ZNF165 are testis antigens, germline genes that are cancer-specific and are not expressed in healthy adult tissues. RASAL3 and RASIP1 are RAS GTPase-activating proteins that play a role in an important regulation pathway, often disturbed in cancer cell lines. BRCA2 is involved in DNA repair mechanisms. Spectra visualization for each modified peptide was created using PDV software² with default parameters. The modified amino acid is colored in the peptides sequence as it appear at the top of the annotated spectra.

FIG. 7 is a schematic representation of the PROtein Modification Integrated Search Engine (PROMISE) pipeline.

FIG. 8 is a schematic representation indicating PTMs as an additional regulatory layer modulating antigen presentation and recognition.

FIG. 9 is a flowchart of an exemplary process for generating a modified sequence dataset storing an indication of binding motifs defined by multiple PTM and corresponding sequence, in accordance with some embodiments of the present invention.

FIG. 10 is a flowchart of an exemplary process for generating an ML model using the modified sequence dataset, in accordance with some embodiments of the present invention.

FIG. 11 is a flowchart of an exemplary process for using the ML model trained using the modified sequence dataset, in accordance with some embodiments of the present invention.

FIG. 12 is a block diagram of a system for generating the modified sequence dataset and/or training the ML model on the modified sequence dataset and/or using the ML model trained on the modified sequence dataset, in accordance with some embodiments of the present invention.

FIGS. 13A-P demonstrates PTM-HLA haplotype motif extracted from the mono-allelic dataset. HLA haplotype motifs from NetMHCpan are presented at the top of the page, followed by the histogram of the site distribution for each identified modification type. The histogram represents the modified amino acid frequency in each position (red) compared to the unmodified amino acid background (grey). Each histogram contains positions 1-7 from the N-terminus and the C-terminus and the preceding position (C-1). Overall, 9 mer epitopes are presented naturally with all their positions, positions 7 and C-1 are identical for 8 mer epitopes and peptides longer than 9 are truncated accordingly.

FIG. 14 is a schematic representation demonstrating the search of ubiquitin tail on endogenous HLA peptides defines any tail length as a variable mass shift.

### DESCRIPTION OF SPECIFIC EMBODIMENTS OF THE INVENTION

The present invention, in some embodiments thereof, relates to agents binding modified antigen dependent peptides and use of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

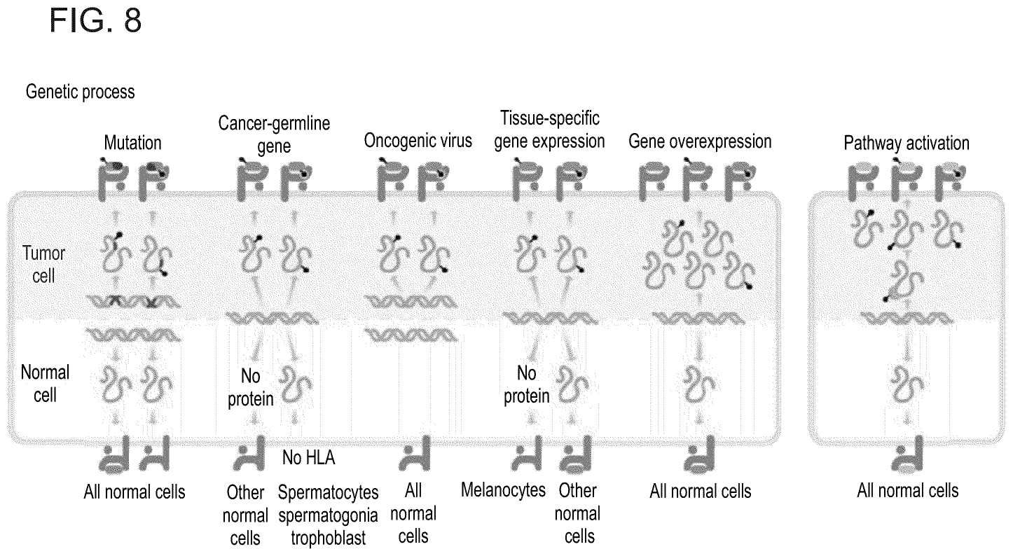

Targeting tumor antigens that are presented by MHC molecules (termed human leukocyte antigens (HLA) in human) holds great promise for cancer T cell therapies and immunotherapies. Typically, antigenic peptides are classified by their genetic origin, including mutations, cancer-germline genes expressed outside of their biological context, oncogenic virus genes, genes with highly tissue specific expression patterns, or overexpression of genes with low endogenous expression (FIG. 8, left block). In recent years, post-translational modifications (PTMs) have also been reported to modulate antigen presentation and recognition (FIG. 8, right block).

As is illustrated hereinunder and in the examples section, which follows, the present inventors developed a PROtein Modification Integrated Search Engine (PROMISE) in order to address the challenges and examine the potential landscape of modified peptides that are presented by MHC in a systematic and unbiased manner allowing rapid and combinatorial detection of multiple PTMs without prior biochemical enrichment (Example 1 hereinbelow). Utilizing this novel computational pipeline the present inventors uncovered and characterized HLA-bound PTM peptides across 210 samples including patient-derived tumor samples and cancer cell lines (Example 2 hereinbelow). Further, the present inventors revealed thousands of modified peptides which are expressed on cancer cells, creating cancer type-specific signatures (Example 3 hereinbelow). Furthermore, some of the identified modified peptides presented by the HLA molecules reside within known cancer-associated antigens or cancer driver genes. In addition, some of the identified peptides comprised remnants from ubiquitin and ubiquitin-like (UBL) modifiers, an observation never disclosed before. By systematic analysis of the locations of peptide modifications on specific HLA, combined with structural 3D modeling and HLA-binding assays, the present inventors further uncovered PTM-driven motifs across many haplotypes, in many cases altering peptide binding or the T cell recognition region of the peptide (Examples 2-3 hereinbelow).

In addition, using this methodology, the present inventors have identified novel HLA-I bound peptides presented on cancerous cells (Example 4 hereinbelow).

Taken together, the present teachings have identified several HLA-restricted modified and un-modified peptides that can be used e.g. as targets for cancer therapy.

Alternatively or additionally, these modified and un-modified peptides can be used as therapeutics per-se as e.g. anti-cancer vaccines.

Thus, according to an aspect of the present invention, there is provided an agent capable of specifically binding an MHC presented peptide comprising a post translational modification (PTM), wherein said peptide amino acid sequence is selected from the group consisting of SEQ ID NO: 1-10746, 10817, 10819, 10820, 10823, 10824, 10826 and 10827 having the corresponding modification according to Table 3 hereinbelow, and wherein said agent does not bind a peptide having the same amino acid sequence as said peptide but does not comprise said modification.

According to an additional or an alternative aspect of the present invention, there is provided an agent capable of binding an MHC presented peptide, wherein said peptide comprises a ubiquitin or a ubiquitin-like (UBL) modifier tail, and wherein said agent does not bind a peptide having the same amino acid sequence as said peptide but does not comprise said tail.

According to an additional or an alternative aspect of the present invention, there is provided an agent capable of specifically binding an MHC presented peptide selected from the group consisting of SEQ ID NO: 10747-10816 and 10822.

As used herein, the term “post-translational modification (PTM)” refers to a chemical modification naturally added to an amino acid residue of a protein or a peptide following its translation. Non-limiting Examples of a post-translational modification include acetylation, amidation, deamidation, alkylation, butyrylation, glycosylation, malonylation, hydroxylation, iodination, nucleotide addition, oxidation, phosphorylation, sulfation, succinylation, ubiquitination, myristolyation, palmitoylation, isoprenylation, methylation, citrullination, sumoylation, cysteinylation.

It will be appreciated that, the post-translation modification can be added synthetically to a peptide.

According to specific embodiments, the PTM is selected from the group of modifications listed in Table 2 hereinbelow.

According to specific embodiments, the modified peptide is selected from the group of peptides listed in Table 3 hereinbelow, wherein each possibility represents a separate embodiment of the present invention.

According to specific embodiments, the modified peptide amino acid sequence is selected from the group of consisting of SEQ ID NO: 1-10746, 10817, 10819, 10820, 10823, 10824, 10826 and 10827 having the corresponding modification according to Table 3 hereinbelow, wherein each possibility represents a separate embodiment of the present invention.

According to specific embodiments, the modified peptide amino acid sequence is selected from the group of consisting of SEQ ID NO: 1-209 and 10819 having the corresponding modification according to Table 3 hereinbelow, wherein each possibility represents a separate embodiment of the present invention.

According to specific embodiments, the modified peptide amino acid sequence is selected from the group of consisting of SEQ ID NO: 210-943 having the corresponding modification according to Table 3 hereinbelow, wherein each possibility represents a separate embodiment of the present invention.

According to specific embodiments, the modified peptide amino acid sequence is selected from the group of consisting of SEQ ID NO: 944-1117 and 10820 having the corresponding modification according to Table 3 hereinbelow, wherein each possibility represents a separate embodiment of the present invention.

According to specific embodiments, the modified peptide amino acid sequence is selected from the group of consisting of SEQ ID NO: 1118-1691 and 10817 having the corresponding modification according to Table 3 hereinbelow, wherein each possibility represents a separate embodiment of the present invention.

According to specific embodiments, the modified peptide amino acid sequence is selected from the group of consisting of SEQ ID NO: 1692-8276 having the corresponding modification according to Table 3 hereinbelow, wherein each possibility represents a separate embodiment of the present invention.

According to specific embodiments, the modified peptide amino acid sequence is selected from the group of consisting of SEQ ID NO: 8277-8897 having the corresponding modification according to Table 3 hereinbelow, wherein each possibility represents a separate embodiment of the present invention.

According to a specific embodiment, the PTM comprises a ubiquitin or a ubiquitin-like (UBL) modifier tail.

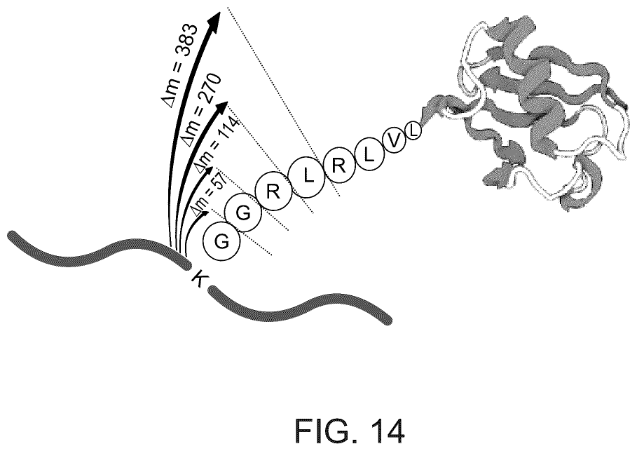

As used herein, the phrase “ubiquitin or a ubiquitin-like (UBL) modifier tail” refers to attachment of ubiquitin (pfam PF00240) or a fragment thereof to a lysine residue of a peptide (see FIG. 14). “A fragment of ubiquitin”, as used herein, refers to at least one amino acid (i.e. at least G) from the C-terminus of ubiquitin.

Thus, according to specific embodiments, the modified peptide amino acid sequence is selected from the group of sequences listed in Table 5 hereinbelow.

According to specific embodiments, the modified peptide amino acid sequence is selected from the group of sequences listed in Table 5 hereinbelow having the corresponding ubiquitin or a ubiquitin-like (UBL) modifier tail according to Table 5 hereinbelow.

According to specific embodiments, the modified peptide amino acid sequence is selected from the group of sequences listed in Table 5 hereinbelow having the corresponding modification according to Table 5 hereinbelow.

According to specific embodiments the modified peptide is further qualified by spectral validation by e.g. mass spectrometry; MHC binding assays such as flow cytometry, immunoprecipitation, immunostaining; and/or reactivity assays such as in-vitro or in-vivo assessment of CD8+ T cells activation, viability and/or killing by methods known in the art.

Lengthy table referenced here
US20240029819A1-20240125-T00001
Please refer to the end of the specification for access instructions.

According to specific embodiments, the peptide is selected from the group of peptides listed in Table 4 hereinbelow, wherein each possibility represents a separate embodiment of the present invention.

According to specific embodiments, the peptide is selected from the group of consisting of SEQ ID NO: 10747-10816 and 10822, wherein each possibility represents a separate embodiment of the present invention.

According to specific embodiments, the peptide is selected from the group of consisting of SEQ ID NO: 10747-10748, wherein each possibility represents a separate embodiment of the present invention.

According to specific embodiments, the peptide is selected from the group of consisting of SEQ ID NO: 10749-10756 and 10822, wherein each possibility represents a separate embodiment of the present invention.

According to specific embodiments, the peptide is as set forth in SEQ ID NO: 10757, wherein each possibility represents a separate embodiment of the present invention.

According to specific embodiments, the peptide is selected from the group of consisting of SEQ ID NO: 10758-10796, wherein each possibility represents a separate embodiment of the present invention.

According to specific embodiments, the peptide is selected from the group of consisting of SEQ ID NO: 10797-10806, wherein each possibility represents a separate embodiment of the present invention.

TABLE 4
list of HLA-1 bound peptides
expressed on tumor cells

| SEQ | | | |
|---|---|---|---|
| ID | | | |
| NO: | Peptide | Gene | Cancer type |
| 10747 | CQICITYI | CARD16 | B-cell-leukemia |
| 10748 | KLNQKRAELK | DNAH3 | B-cell-leukemia |
| 10749 | GDLCRICQM | 7 Mar | Breast |
| 10750 | TQELQQAK | CENPF | Breast |
| 10751 | QAMQFGQLL | | Breast |
| 10752 | QEIDFLQQLY | | Breast |
| 10753 | GELIIWDALDW | WDR41 | Breast |
| 10754 | GYSNGVIN | SMARCA2 | Breast |
| 10755 | QDCAVLQQSSL | HARBI1 | Breast |
| 10756 | KKLLQLKNEN | RASGRP3 | Breast |
| 10757 | HQQAEVFIV | CATIP | Colon |
| 10758 | LPVSICRSCETL | SASH1 | Melanoma |
| 10759 | LTECPEIEICY | PARP14 | Melanoma |
| 10760 | DVIGDEICCW | KCTD10 | Melanoma |
| 10761 | TLQPGCGRPQV | | Melanoma |
| 10762 | THSHTCQCF | ELP1 | Melanoma |
| 10763 | PYVCQVCQF | ZNF280C | Melanoma |
| 10764 | PQLPQSSQL | GLG1 | Melanoma |
| 10765 | DVLGDEICCW | | Melanoma |
| 10766 | TQVIKVLNP | AP1G1 | Melanoma |
| 10767 | AVCGKVKCK | SNX14 | Melanoma |
| 10768 | QSNETALHYF | SELIL | Melanoma |
| 10769 | KLWACNFCF | SEC23B | Melanoma |
| 10770 | LSVAPQQSLVL | BCS1L | Melanoma |
| | LED | | |
| 10771 | VCTIISDPTCE | GPNMB | Melanoma |
| | ITQN | | |
| 10772 | QLLPNSQSFI | TAF4B | Melanoma |
| 10773 | VQQAGQLAR | HPS1 | Melanoma |
| 10774 | SNSTARNVTW | SERPINH1 | Melanoma |
| 10775 | QNLSFGAT | LRRC40 | Melanoma |
| 10776 | LQKLVRIQL | | Melanoma |
| 10777 | TRHDCPVCL | CREBBP | Melanoma |
| 10778 | RTFCKKCGK | RPL36A | Melanoma |
| 10779 | LINNDLYRI | ZCRB1 | Melanoma |
| 10780 | GVSGVCVCK | IGFBP7 | Melanoma |
| 10781 | KMKLKQQRV | | Melanoma |
| 10782 | KSREDCCTKF | GPM6B | Melanoma |
| 10783 | CTDCYSNEY | FHL2 | Melanoma |
| 10784 | THDCCYDHL | PLA2G2D | Melanoma |
| 10785 | THIQQAPAL | RERE | Melanoma |
| 10786 | YCKNKPYPKS | RPLIOL | Melanoma |
| | RFC | | |
| 10787 | TNAVIFSQKI | ORC2 | Melanoma |
| 10788 | TQLTMNVPFQ | SLC25A28 | Melanoma |
| 10789 | TRCGCVTML | MED16 | Melanoma |
| 10790 | GPHQQSHQES | FLG | Melanoma |
| | ARD | | |
| 10791 | FLEDVLNEIQ | RARS2 | Melanoma |
| 10792 | GEIICKCGQAW | IFIH1 | Melanoma |
| 10793 | EHCGCYTLL | MYLK | Melanoma |
| 10794 | PEGQPGPWGQAL | FBN3 | Melanoma |
| 10795 | RQNVPRKV | CPXM2 | Melanoma |
| 10796 | LYAKCIPCI | FCF1 | Melanoma |
| 10797 | TGGDNQLLLY | PDE10A | Meningioma |
| 10798 | YSQEIENHY | NWD2 | Meningioma |
| 10799 | YHCHCRIVL | NEU1 | Meningioma |
| 10800 | KTNISHNGTY | FCGRIB | Meningioma |
| 10801 | TDQVIQNEMP | | Meningioma |
| 10802 | QSDCSCSTV | TYROBP | Meningioma |
| 10803 | RSLSNSTARN | SERPINH1 | Meningioma |
| | VTW | | |
| 10804 | RVQDVACRCR | STABI | Meningioma |
| 10805 | PNNHIGISF | FLNB | Meningioma |
| 10806 | LAQAVSTQLY | FAM120C | Meningioma |
| 10807 | KICCGIIYK | SINHCAF | B-cell-leukemia, |
| | | | Melanoma |
| 10808 | NIHSIVVQV | PCDH15 | Breast, |
| | | | Glioblastoma, |
| | | | Meningioma, |
| | | | Melanoma |
| 10809 | GADGNIFVEN | | Glioblastoma, |
| | | | Melanoma |
| 10810 | QSNEMVLQ | | Glioblastoma, |
| | | | Meningioma |
| 10811 | STNHTVNHTY | GPNMB | Meningioma, |
| | | | Melanoma |
| 10812 | FTDCYKCFY | PMF1 | Meningioma, |
| | | | Melanoma |
| 10813 | TGQILKQTY | CSH2 | Meningioma, |
| | | | Melanoma |
| 10814 | CSDEASGCHY | NR3C1 | Meningioma, |
| | | | Melanoma |
| 10815 | TRCGCVTML | MED16 | Meningioma, |
| | | | Melanoma |
| 10816 | KKDSKNDNFK | NUP153 | Meningioma, |
| | | | Melanoma |
| 10822 | RPLDEKDTSM | SPAG9 | Breast |

TABLE 5
list of modified HLA-1 bound peptides having
an ubiquitin or ubiquitin-like (UBL)
modifier tail expressed on tumor cells

| SEQ | | |
|---|---|---|
| ID | | |
| NO: | Peptide | Peptide modificaiton |
| 15 | GTDEHVVCK | 8,C,Cysteinylation; |
| | | 9,K,Ubiquitylation |
| 22 | GTDEHVVCK | 9,K,Ubiquitylation |
| 52 | SVFDNSIKTFGV | 8,K,Ubiquitylation |
| 57 | DIIKHIVAK | 4,K,Ubiquitylation; |
| | | 5,H,Oxidation |
| 75 | DIIKHIVAK | 4,K,Ubiquitylation |
| 123 | KKGWPKGKS | 6,K,Oxidation; |
| | | 8,K,Ubiquitylation |
| 125 | AQCGKAFPK | 5,K,Ubiquitylation |
| 127 | NTQIFKTNTQTYREN | 3,Q,Deamidation; |
| | | 6,K,Ubiquitylation |
| 148 | NTQIFKTNTQTYREN | 6,K,Ubiquitylation |
| 150 | SSCGKFQTK | 5,K,Ubiquitylation |
| 181 | SLKYPDENGFDAFLK | 3,K,Ubiquitylation; |
| | | 8,N,Deamidation |
| 185 | RHRKKLYV | 3,R,Citrullination; |
| | | 5,K,Ubiquitylation |
| 209 | SLKYPDENGEDAFLK | 3,K,Ubiquitylation |
| 212 | RPKDYEVDATLKSLN | 12,K,Ubiquitylation; |
| | | 15,N,Deamidation |
| 213 | RPKDYEVDATLKSLN | 12,K,Ubiquitylation |
| 243 | SAQGSDVSLTACKV | 12,C,Cysteinylation; |
| | | 13,K,Ubiquitylation |
| 245 | TTAFQYIIDNKGIDS | 10,N,Deamidation; |
| | | 11,K,Ubiquitylation |
| 255 | SAQGSDVSLTACKV | 13,K,Ubiquitylation |
| 256 | TTAFQYIIDNKGIDS | 11,K,Ubiquitylation |
| 264 | FIDLLHDK | 4,L,Methylation; |
| | | 8,K,Ubiquitylation |
| 288 | GHQQLYWSHPRKFGQ | 12,K,Ubiquitylation |
| 291 | KSPAKPKAV | 3,P,Oxidation; |
| | | 5,K,Oxidation; |
| | | 6,P,Oxidation; |
| | | 7,K,Ubiquitylation |
| 345 | KTDQAQKAEGAGDAK | 1,K,Ubiquitylation; |
| | | 3,D,Methylation |
| 347 | YKDPLFKKLEQLKEV | 2,K,FAT10; |
| | | 7,K,FAT10; |
| | | 8,K,Ubiquitylation |
| 350 | VAKKKDKVKKGGP | 10,K,Ubiquitylation |
| 360 | SKMEFMTI | 2,K,Ubiquitylation |
| 403 | DGTFQKWASVVVPSG | 6,K,Ubiquitylation |
| 448 | AVMDSDTTGKLGF | 10,K,Ubiquitylation |
| 457 | DASKGDDLLPAGTED | 1,D,Methylation; |
| | | 4,K,Ubiquitylation |
| 469 | QVQLVESGGGLVKPG | 13,Q,Deamidation; |
| | | 3,K,Ubiquitylation |
| 475 | QPLDGLKTY | 1,Q,Methylation; |
| | | 7,K,Ubiquitylation |
| 482 | DATKGDDLLPAGTED | 4,K,Ubiquitylation |
| 485 | KQTALVELVKHK | 10,K,Ubiquitylation |
| 490 | KVQWKVDNALQSGNS | 1,K,Ubiquitylation; |
| | | 3,Q,Methylation |
| 503 | EDFDVKTY | 6,K,Ubiquitylation |
| 505 | RYISKYELDKAFS | 1,R,Citrullination; |
| | | 5,K,Ubiquitylation |
| 519 | SFDVVTKCV | 7,K,Ubiquitylation |
| 524 | GENIKQIF | 5,K,Ubiquitylation; |
| | | 6,Q,Methylation |
| 530 | LFLLPSLK | 8,K,Ubiquitylation |
| 539 | KESTLHLVL | 1,K,Ubiquitylation |
| 548 | KDLVQDCGF | 1,K,Ubiquitylation |
| 558 | VEAKDCLNVL | 4,K,Ubiquitylation |
| 562 | PGLARQAPKPRK | 11,R,Methylation; |
| | | 12,K,Ubiquitylation |
| 566 | PGKNVVTTL | 3,K,Ubiquitylation |
| 666 | QVQLVESGGGLVKPG | 3,K,Ubiquitylation |
| 685 | DLVEGGKYEFR | 7,K,Ubiquitylation |
| 690 | KTAKPKAAK | 4,K,Ubiquitylation; |
| | | S,P,Oxidation |
| 694 | AAQTKATFLKLAGPQ | 10,K,Ubiquitylation; |
| | | S,T,Phosphorylation; |
| | | 7,K,Ubiquitylation |
| 698 | EEEKIVKKL | 4,K,Ubiquitylation |
| 721 | VYCGKKAQLNI | 5,K,Ubiquitylation |
| 732 | VNVVPTFGKKKGPN | 10,K,Sumoylation; |
| | | 11,K,Ubiquitylation |
| 738 | NPGGYVAYSKAATVT | 10,K,Ubiquitylation |
| 741 | KAMKALESI | 4,K,Ubiquitylation |
| 751 | KEKFEKDKSEKED | 2,B,Methylation; |
| | | 6,K,Ubiquitylation; |
| | | 8,K,Ubiquitylation |
| 765 | PNMVTPGHACTQK | 3,K,Ubiquitylation |
| 766 | PGVLDRMMKKLDTNS | 10,K,Ubiquitylation; |
| | | 14,N,Deamidation |
| 773 | BYGGSVTGATCK | 13,K,Ubiquitylation |
| 793 | KKEGKIYRL | 5,K,Ubiquitylation |
| 794 | KKKKQVLKFTLD | 3,K,Ubiquitylation |
| 796 | KIGAVVGGVL | 1,K,Ubiquitylation |
| 826 | KVVSETNDTKVLRH | 1,K,Ubiquitylation; |
| | | 7,N,Deamidation |
| 844 | GSPVKAGVETTKPSK | 12,K,Ubiquitylation; |
| | | 15,K,Methylation |
| 847 | RQKDVKDGKYSQV | 9,K,Ubiquitylation |
| 927 | PGVLDRMMKKLDINS | 10,K,Ubiquitylation |
| 939 | KVVSETNDTKVLRH | 1,K,Ubiquitylation |
| 972 | TEEEKNFKA | 8,K,Ubiquitylation |
| 990 | STDKQMGY | 4,K,Ubiquitylation |
| 1045 | STDKQMGY | 4,K,Ubiquitylation |
| 1052 | APAQKAPAPKASGKK | 14,K,Methylation; |
| | | 15,K,Ubiquitylation |
| 1070 | KAMEEKLEA | 6,K,Ubiquitylation |
| 1079 | KGGKGLGKGGAK | 4,K,Ubiquitylation |
| 1080 | KGGKGLGK | 4,K,Ubiquitylation |
| 1090 | RGKAGKGLGKGGAK | 1,R,Citrullination; |
| | | 3,K,Ubiquitylation; |
| | | 6,K,Ubiquitylation |
| 1118 | FVTPLTSMVVTKPDD | 12,K,Ubiquitylation; |
| | | 14,D,Methylation |
| 1120 | FVTPLTSMVVTKPED | 12,K,Ubiquitylation |
| 1140 | FVTPLTSMVVTKPED | 8,K,Ubiquitylation |
| 1158 | TPGKKGAAIPAKGAK | 15,K,Ubiquitylation |
| 1163 | AGAGKVTKSAQKAQK | 14,Q,Methylation; |
| | | 15,K,Ubiquitylation |
| 1164 | HFDLSHGSAQVKGHG | 12,K,Ubiquitylation; |
| | | 14,H,Methylation |
| 1175 | SLIQTKCADDAMTL | 6,K,Ubiquitylation |
| 1182 | SNKGAIIGLMVGGVV | 2,N,Deamidation; |
| | | 3,K,Ubiquitylation |
| 1189 | IGFPGPPGPKG | 10,K,Ubiquitylation |
| 1195 | SNKGAIIGLMVGGVV | 3,K,Ubiquitylation |
| 1205 | KGAAIPAKGAKNGKN | 1,K,Ubiquitylation |
| 1206 | VLDELKNMKC | 10,K,Ubiquitylation; |
| | | 9,C,Cysteinylation |
| 1216 | SLIQTKCADDAMTL | 12,K,Ubiquitylation |
| 1217 | SPNIVIALAGNKADL | 12,K,Ubiquitylation; |
| | | 14,D,Methylation |
| 1227 | VLDELKNMKC | 10,K,Ubiquitylation |
| 1233 | KGAAIPAKGAKNGKN | 12,K,Ubiquitylation; |
| | | 1,N,Deamidation |
| 1238 | LAFEGTPEQK | 10,K,Ubiquitylation |
| 1259 | PVPEPEPEPEPEPVK | 13,P,Oxidation; |
| | | 15,K,Ubiquitylation |
| 1266 | FMGPLKKDRIAKEE | 12,K,Ubiquitylation; |
| | | 13,E,Methylation |
| 1288 | KGAAIPAKGAKNGKN | 12,K,Ubiquitylation |
| 1293 | VGSNKGAIIGLMVGG | 4,N,Deamidation; |
| | | 5,K,Ubiquitylation |
| 1305 | MNWNKGGPGTKR | 11,K,Ubiquitylation; |
| | | 1,M,Acetylation |
| 1312 | FTKFNADEFEDMVAE | 3,K,Ubiquitylation |
| 1314 | GGYVKLFPNSLDQTD | 5,K,Ubiquitylation |
| 1341 | DKPDMGEIASFDKAK | 2,K,Ubiquitylation |
| 1345 | KRTKKVGIVGKYG | 1,K,Ubiquitylation; |
| | | 2,R,Methylation |
| 1361 | VGSNKGAIIGLMVGG | 5,K,Ubiquitylation |
| 1365 | GYVKLFPNSLDQTDM | 4,K,Ubiquitylation |
| 1376 | IAGFLQKN | 6,Q,Deamidation; |
| | | 7,K,Ubiquitylation |
| 1383 | KRAKEAAEQDVEKKK | 4,K,Ubiquitylation; |
| | | 5,E,Methylation |
| 1387 | EKKQPVDLGLLEEDD | 2,K,Oxidation; |
| | | 3,K,Ubiquitylation |
| 1389 | KRTKKVGIVGKYG | 2,R,Methylation; |
| | | 4,K,Ubiquitylation |
| 1392 | EDDDVDTKKQKTDED | 10,Q,Deamidation; |
| | | 11,K,Ubiquitylation |
| 1403 | SGKVTFPK | 3,K,Ubiquitylation |
| 1420 | AKPEPVIEEVDLANL | 2,K,Ubiquitylation; |
| | | 4,E,Methylation |
| 1427 | KEADLAAQEEAAKK | 1,K,Ubiquitylation |
| 1431 | VLCPPPVKK | 8,K,Ubiquitylation |
| 1446 | VRKPVVSTISKGGYL | 11,K,Ubiquitylation; |
| | | 15,L,Methylation |
| 1490 | IAGFLQKN | 7,K,Ubiquitylation |
| 1492 | EDDDVDTKKQKTDED | 11,K,Ubiquitylation |
| 1517 | EDDDVDTKKQKTDED | 10,K,Ubiquitylation; |
| | | 11,Q,Deamidation; |
| | | 9,K,Ubiquitylation |
| 1539 | AEKCSQSNNQF | 3,K,Ubiquitylation |
| 1554 | NSQTKPGGLFGTSSF | 5,K,Ubiquitylation |
| 1564 | QKLSELDDRADALQ | 1,Q,Deamidation; |
| | | 2,K,Ubiquitylation |
| 1573 | KLHTGVKPH | 3,H,Oxidation; |
| | | 7,K,Ubiquitylation; |
| | | 9,H,Oxidation |
| 1575 | KKTATAVAHCK | 10,C,Cysteinylation; |
| | | 11,K,Ubiquitylation |
| 1576 | MKTVQKKCEKLQKNK | 10,K,Ubiquitylation; |
| | | 12,K,Ubiquitylation; |
| | | 13,K,Ubiquitylation; |
| | | 14,Q,Methylation; |
| | | 15,K,Ubiquitylation; |
| | | 2,N,Methylation; |
| | | 7,K,Ubiquitylation |
| 1589 | KHPPENIIDGNPETF | 1,K,Ubiquitylation; |
| | | 3,P,Oxidation; |
| | | 4,P,Oxidation |
| 1590 | LHYDPNKRIS | 7,K,Ubiquitylation; |
| | | 8,R,Methylation |
| 1605 | SNYGPMKSGNF | 7,K,Ubiquitylation |
| 1620 | RILPKPTRK | 5,K,Ubiquitylation |
| 1676 | EDDDVDTKKQKTDED | 10,K,Ubiquitylation; |
| | | 9,K,Ubiquitylation |
| 1683 | QKLSELDDRADALQ | 2,K,Ubiquitylation |
| 1686 | KLHTGVKPH | 7,K,Ubiquitylation |
| 1687 | KKTATAVAHCK | 11,K,Ubiquitylation |
| 1703 | KVNVDEVGGEALGRL | 1,K,Ubiquitylation |
| 1712 | TLVQTKGTGASGSFK | 6,K,Ubiquitylation |
| 1718 | PAYHSSLMDPDTKLI | 13,K,Ubiquitylation |
| 1759 | HPKYKTEL | 3,K,Ubiquitylation |
| 1788 | IPGHLNSYTIKGLKP | 14,K,Ubiquitylation |
| 1811 | GSEMVVAGKLQDRGP | 11,K,Ubiquitylation; |
| | | 9,Q,Deamidation |
| 1821 | GSEMVVAGKLQDRGP | 11,K,Ubiquitylation |
| 1860 | PAYHSSLMDPDTKLI | 13,K,Ubiquitylation |
| 1862 | PAYHSSLMDPDTKLI | 8,K,Ubiquitylation |
| 1896 | GERIEKVEHSDLSFS | 6,K,Ubiquitylation |
| 1900 | SSHKTFRIKRFL | 10,K,Ubiquitylation; |
| | | 9,R,Methylation |
| 1901 | SSHKTFRIKRFL | 9,K,Ubiquitylation; |
| | | 10,R,Methylation |
| 1963 | KYPDRVPVI | 1,K,Ubiquitylation |
| 2013 | KDSTYSLSSTLTLSK | 13,L,Methylation; |
| | | 15,K,Ubiquitylation |
| 2064 | KRGVAIAR | 1,K,Ubiquitylation; |
| | | 2,R,Citrullination |
| 2103 | GILNVSAVDKSTGKE | 14,K,Ubiquitylation |
| 2154 | YASTAKCL | 6,K,Ubiquitylation |
| 2158 | KIVPFFKL | 2,1,Methylation; |
| | | 7,K,Ubiquitylation |
| 2169 | QTVDLFEGKDMAA | 11,K,Ubiquitylation |
| 2171 | TYMRIYKKGDIVDIK | 5,1,Methylation; |
| | | 7,K,Ubiquitylation |
| 2176 | YLRGGAGVGSMTKIY | 13,K,Ubiquitylation |
| 2223 | DVKKEPLGR | 3,K,Ubiquitylation |
| 2234 | EGLTFQMKKNAEELK | 14,L,Methylation; |
| | | 15,K,Ubiquitylation |
| 2247 | QKKVEELEGEITT | 1,Q,Deamidation; |
| | | 2,K,Ubiquitylation |
| 2255 | TKRKWEAVHAAEQRR | 2,K,Dimethyl; |
| | | 3,R,Dimethyl; |
| | | 4,K,Ubiquitylation |
| 2305 | QKKVEELEGEITT | 2,K,Ubiquitylation |
| 2315 | ARGPKKHLKRV | 10,K,Ubiquitylation; |
| | | 9,R,Methylation |
| 2317 | ARGPKKHLKRV | 9,K,Ubiquitylation; |
| | | 10,R,Methylation |
| 2330 | RGKFAVVR | 3,K,Ubiquitylation |
| 2331 | VYSRHPAENGKSNFL | 11,K,Ubiquitylation |
| 2344 | TFQKWAAVVVPSG | 4,K,Ubiquitylation |
| 2435 | DLQKKLVPFATELHE | 4,K,Ubiquitylation |
| 2554 | FKRGADPGMPEPTVL | 2,K,Ubiquitylation |
| 2557 | SSHKTFRIKRFL | 12,K,Ubiquitylation; |
| | | 9,L,Methylation |
| 2559 | SSHKTFRIKRFL | 9,K,Ubiquitylation; |
| | | 12,L,Methylation |
| 2584 | RSWTAADMAAQITKR | 14,K,Ubiquitylation; |
| | | 15,R,Methylation |
| 2587 | KGKQSISK | 3,K,Ubiquitylation |
| 2682 | HKAVLTIDEKGTEA | 10,K,Ubiquitylation; |
| | | 13,E,Methylation |
| 2686 | PEAAVGLLKGTAL | 2,E,Methylation; |
| | | 9,K,Ubiquitylation |
| 2687 | RGKTYISK | 1,R,Methylation; |
| | | 3,K,Ubiquitylation |
| 2693 | RKLFYVHY | 2,K,Ubiquitylation |
| 2703 | KQKTFIVK | 2,Q,Methylation; |
| | | 3,K,Ubiquitylation |
| 2708 | GTNKVASQK | 4,K,Ubiquitylation |
| 2724 | ERQSAEDYEKEE | 10,E,Methylation; |
| | | 1,D,Methylation; |
| | | 7,K,Ubiquitylation |
| 2726 | EESEKLSKMSSLLE | 11,K,Ubiquitylation; |
| | | 5,S,Phosphorylation; |
| | | 7,K,Ubiquitylation; |
| | | 8,S,Phosphorylation |
| 2748 | KAVDKKAAGAGKVTK | 1,K,Dimethyl; |
| | | 5,K,Dimethyl; |
| | | 6,K,Ubiquitylation |
| 2761 | KGRLSKDDIDRMVQE | 1,K,Ubiquitylation; |
| | | 3,R,Citrullination |
| 2762 | DVKKEPLGR | 3,K,Ubiquitylation |
| 2867 | GQKDSYVGDEAQSKR | 12,Q,Deamidation; |
| | | 14,K,Ubiquitylation |
| 2907 | AEIRLVSKDGKSKGI | 13,K,Ubiquitylation; |
| | | 15,I,Methylation |
| 2919 | GTRYVQKGEYRTNPE | 6,Q,Deamidation; |
| | | 7,K,Ubiquitylation |
| 2930 | ILYGKIIHL | 5,K,Ubiquitylation |
| 2936 | FLEQVHQGIKGM | 10,1,Methylation; |
| | | 9,K,Ubiquitylation |
| 2954 | KLRKRLAPL | 2,L,Methylation; |
| | | 4,K,Ubiquitylation; |
| | | 6,L,Methylation; |
| | | 9,L,Methylation |
| 2955 | PNNLKPVVAEFYGSK | 4,L,Methylation; |
| | | 5,K,Ubiquitylation |
| 2975 | GKQLEDGRTL | 2,K,Ubiquitylation |
| 2976 | IAEERDKRLAAKQSS | 12,K,Ubiquitylation |
| 3121 | GQKDSYVGDEAQSKR | 14,K,Ubiquitylation |
| 3125 | GTRYVQKGEYRTNPE | 7,K,Ubiquitylation |
| 3136 | KEFTPPVQAAYQKVV | 12,Q,Methylation; |
| | | 13,K,Ubiquitylation |
| 3150 | PDLKLVPPMEEDYPQ | 13,K,Ubiquitylation; |
| | | 4,Y,Phosphorylation |
| 3158 | PEEDKKTYGEIFEKF | 2,E,Methylation; |
| | | S,K,Ubiquitylation |
| 3248 | VKKQKKPLVGKKAAA | 2,K,Ubiquitylation |
| 3259 | SPADKTNVKAAWGKV | 14,K,Ubiquitylation |
| 3282 | TPGAEDKGK | 6,D,Methylation; |
| | | 7,K,Ubiquitylation |
| 3431 | STDVKGCSMY | 5,K,Ubiquitylation |
| 3437 | ETRPAGDGTFQK | 11,Q,Deamidation; |
| | | 12,K,Ubiquitylation |
| 3444 | VIQHFQEKVESLEQE | 12,K,Ubiquitylation; |
| | | 8,L,Methylation |
| 3454 | KYLQAKLTQF | 6,K,Ubiquitylation; |
| | | 7,L,Methylation |
| 3459 | KYPDRVPVI | 1,K,Ubiquitylation |
| 3477 | LGEEKGGASLSPQYV | 1,L,Methylation; |
| | | 5,K,Ubiquitylation |
| 3492 | AVFEWHITKGGNI | 12,K,Ubiquitylation; |
| | | 9,N,Methylation |
| 3500 | TQIFKTNTQTYRESL | 5,K,Ubiquitylation |
| 3518 | KKWGKSKKK | 5,K,Ubiquitylation |
| 3533 | KIFNVAIPRF | 1,K,Ubiquitylation |
| 3542 | TGKTTFVK | 3,K,Ubiquitylation |
| 3555 | TEKLVTSKGDKELRT | 11,K,Ubiquitylation |
| 3556 | VDSKGFDEYMKELGV | 11,K,Ubiquitylation |
| 3574 | GPSVPKMMNLKGNPE | 7,K,Ubiquitylation |
| 3575 | ADADADLEERLKNLR | 12,K,Ubiquitylation; |
| | | 13,N,Deamidation |
| 3583 | ADKTNVKAAWGKVG | 2,D,Methylation; |
| | | 3,K,Ubiquitylation |
| 3584 | GHKPPGSSEPITVKF | 3,K,Ubiquitylation |
| 3592 | KTKDGVREV | 3,K,Ubiquitylation |
| 3597 | KTATAVAHCK | 10,K,Ubiquitylation |
| 3598 | RLAPDYDALDVANKI | 14,K,Ubiquitylation |
| 3599 | RKLVATKL | 2,K,Ubiquitylation |
| 3600 | HFDLSHGSAQVKGH | 10,Q,Methylation; |
| | | 12,K,Ubiquitylation |
| 3609 | RAQDLPLKK | 8,K,Ubiquitylation |
| 3611 | RSHTGKYSI | 1,R,Dimethyl; |
| | | 6,K,Ubiquitylation |
| 3618 | GTKDTVSTGLTGAVN | 3,K,Ubiquitylation; |
| | | 4,D,Methylation |
| 3619 | GTKDTVCSGVTGAAN | 3,K,Ubiquitylation; |
| | | 4,D,Methylation |
| 3627 | FKRGADPGMPEPTVL | 2,K,Ubiquitylation |
| 3636 | VTATALKT | 7,K,Ubiquitylation |
| 3641 | PDGIGKLKKL | 6,K,Ubiquitylation; |
| | | 8,K,Ubiquitylation; |
| | | 9,K,Ubiquitylation |
| 3669 | PFKLFEIDPTSGVVS | 3,K,Ubiquitylation |
| 3942 | ETRPAGDGTFQK | 12,K,Ubiquitylation |
| 3963 | ADADADLEERLKNLR | 12,K,Ubiquitylation |
| 4023 | VHKAVLTIDEKGTEA | 10,E,Methylation; |
| | | 11,K,Ubiquitylation |
| 4039 | VLNTNIDGRRKI | 10,R,Methylation; |
| | | 11,K,Ubiquitylation |
| 4054 | RLKNEGATVK | 3,K,Ubiquitylation; |
| | | 4,N,Deamidation |
| 4063 | SDSARSKTL | 7,K,Ubiquitylation |
| 4075 | SLSKLGDVYVNDAFG | 2,L,Methylation; |
| | | 4,K,Ubiquitylation |
| 4080 | MSRYELKLAIPEGKQ | 6,L,Methylation; |
| | | 7,K,Ubiquitylation |
| 4095 | RLKYALTGDEVK | 3,K,Ubiquitylation |
| 4098 | RKTIVVNF | 2,K,Ubiquitylation |
| 4111 | QQAADKYLYVDKNFI | 6,K,Ubiquitylation |
| 4121 | TKPPSLQWAW | 2,K,Ubiquitylation |
| 4126 | TLGSGVTGAAKVA | 11,K,Ubiquitylation |
| 4131 | TGKSLLHLH | 3,K,Ubiquitylation |
| 4142 | LSAAKSKPIIA | 7,K,Ubiquitylation |
| 4148 | LTDITKGVQY | 6,K,Ubiquitylation |
| 4157 | LTELCKQKPADPL | 6,K,Ubiquitylation; |
| | | 8,K,Sumoylation |
| 4160 | SQVMREWEEAERQAK | 4,K,Ubiquitylation |
| 4183 | TAADTAAQISKR | 11,K,Ubiquitylation; |
| | | 12,R,Citrullination |
| 4184 | TAAPAVAETPDIKLF | 13,K,Ubiquitylation |
| 4185 | TAFQYIIDNKGIDSD | 10,K,Ubiquitylation; |
| | | 12,I,Methylation |
| 4211 | GTVRIGVAK | 9,K,Ubiquitylation |
| 4217 | HQPHKVTQYKKGKDS | 10,K,Dimethyl; |
| | | 11,K,Ubiquitylation; |
| | | 13,K,Dimethyl |
| 4231 | FVKVVKNKAYFKRYQ | 3,K,Ubiquitylation |
| 4242 | KKKEADAIKL | 3,K,Ubiquitylation; |
| | | 6,D,Methylation |
| 4246 | KKAKAPGLSSK | 4,K,Ubiquitylation; |
| | | 6,P,Oxidation |
| 4247 | KISSKNVQIK | 5,K,Ubiquitylation |
| 4255 | KLEKAKAKELATKLG | 1,K,Dimethyl; |
| | | 4,K,Ubiquitylation |
| 4257 | KMVDQLFCKK | 9,K,Ubiquitylation |
| 4265 | KGQKYFDSGDYNMAK | 3,Q,Methylation; |
| | | 4,K,Ubiquitylation |
| 4278 | KFIDTTSKF | 1,K,Ubiquitylation |
| 4354 | DDGKIVIFQSKPEIQ | 1,D,Methylation; |
| | | 4,K,Ubiquitylation |
| 4358 | DRTFQKWAAVVVPSG | 6,K,Ubiquitylation |
| 4788 | RLKNEGATVK | 3,K,Ubiquitylation |
| 4842 | AVGKPHGIAI | 4,K,Ubiquitylation |
| 4867 | RLLNINPNK | 4,N,Methylation; |
| | | 8,N,Methylation; |
| | | 9,K,Ubiquitylation |
| 4868 | KYRKVLQL | 3,R,Citrullination; |
| | | 4,K,Ubiquitylation; |
| | | 7,Q,Deamidation |
| 4879 | HFDLSHGSAQVKGH | 12,K,Ubiquitylation |
| 4889 | TGKQLALLK | 3,K,Ubiquitylation; |
| | | 5,L,Methylation |
| 4907 | RPWKKHSTF | 4,K,Ubiquitylation |
| 4917 | THVTKSLHSI | 5,K,Ubiquitylation |
| 4919 | AYKAIPVAQDLNAPS | 3,K,Ubiquitylation |
| 4935 | RLKVKGDLAM | 3,K,Ubiquitylation |
| 4936 | RLKVKGDLAM | 3,K,Ubiquitylation |
| 4945 | KPLPQPVF | 1,K,Ubiquitylation; |
| | | 5,Q,Deamidation |
| 4970 | QGPKQASGAAAA | 1,Q,Methylation; |
| | | 4,K,Ubiquitylation |
| 4972 | IEVDGKQVEL | 6,K,Ubiquitylation |
| 4984 | HVPGGGNVKIDSQKL | 14,K,Ubiquitylation |
| 5005 | HKPGGGDVKIESQKL | 14,K,Ubiquitylation |
| 5014 | TNVKAAWGKV | 9,K,Ubiquitylation |
| 5017 | RKGTDDSMTL | 2,K,Ubiquitylation |
| 5024 | TPKTPKGPSSVEDIK | 14,1,Methylation; |
| | | 15,K,Ubiquitylation |
| 5056 | SLKDEVLKIMPV | 2,L,Methylation; |
| | | 3,K,Ubiquitylation |
| 5065 | FKHIAKPGWK | 2,K,Ubiquitylation; |
| | | 6,K,Dimethyl |
| 5066 | SKSPDPYRL | 2,K,Ubiquitylation; |
| | | 3,S,Phosphorylation |
| 5095 | DVFRDPALKR | 9,K,Ubiquitylation |
| 5100 | SKAVVQVF | 2,K,Ubiquitylation |
| 5112 | SHEDPEVKF | 8,K,Ubiquitylation |
| 5129 | EGKATSTTEL | 1,E,Methylation; |
| | | 3,K,Ubiquitylation |
| 5130 | EGKFPSAA | 3,K,Ubiquitylation |
| 5132 | EGKLESLEL | 3,K,Ubiquitylation |
| 5134 | SQLHKENL | 5,K,Ubiquitylation; |
| | | 6,E,Methylation |
| 5135 | SQKDILEEKRAVPDR | 3,K,Ubiquitylation |
| 5140 | EGSEIVVAGRIADNK | 14,N,Methylation; |
| | | 15,K,Ubiquitylation |
| 5142 | KRNKQTYSTEPNNLK | 1,K,Dimethyl; |
| | | 2,R,Dimethyl; |
| | | 4,K,Ubiquitylation |
| 5157 | EPKFLDEPYEAIVPE | 3,K,Ubiquitylation |
| 5162 | EPTKSAPAPKKGSK | 10,K,Oxidation; |
| | | 11,P,Oxidation; |
| | | 14,K,Oxidation; |
| | | 4,K,Ubiquitylation; |
| | | 7,K,Oxidation |
| 5171 | TDLLLKLL | 6,K,Ubiquitylation |
| 5180 | DIAPTLTLYVGKKQL | 12,K,Sumoylation; |
| | | 13,K,Ubiquitylation |
| 5185 | DIKCVLNEGMPIYR | 3,K,Ubiquitylation |
| 5186 | SAQGSDVSLTACKV | 12,C,Oxidation; |
| | | 13,K,Ubiquitylation |
| 5193 | KRYKSIVKY | 4,K,Ubiquitylation |
| 5226 | SGDTTAPKKTSF | 9,K,Ubiquitylation |
| 5242 | KVIETQLAK | 1,K,Methylation; |
| | | 9,K,Ubiquitylation |
| 5277 | YLRGGAGVGSMTKIY | 13,K,Ubiquitylation |
| 5297 | KGDKCLLKY | 4,K,Ubiquitylation; |
| | | 5,C,Oxidation |
| 5311 | KSQGVGPIRKV | 1,K,Ubiquitylation; |
| | | 3,Q,Methylation |
| 5313 | KFIDTTSKF | 8,K,Ubiquitylation |
| 5318 | MSRYELKLAIPEGKQ | 3,R,Dimethyl; |
| | | 7,K,Ubiquitylation |
| 5351 | LVDVEPKVKSKKRE | 11,K,Dimethyl; |
| | | 12,K,Ubiquitylation; |
| | | 13,R,Dimethyl |
| 5360 | KGGKLNSAK | 4,K,Ubiquitylation |
| 5372 | KKIKDLPSL | 2,K,Ubiquitylation |
| 5374 | KKPALKKLTLLPAVV | 2,K,Ubiquitylation; |
| | | 6,K,Ubiquitylation; |
| | | 7,K,Acetylation |
| 5386 | YDGKDYIALNEDLRS | 2,D,Methylation; |
| | | 4,K,Ubiquitylation |
| 5462 | PRVLKQVH | 2,R,Citrullination; |
| | | 5,K,Ubiquitylation; |
| | | 6,Q,Deamidation |
| 5469 | LQKKLVPFATELHER | 2,Q,Deamidation; |
| | | 3,K,Ubiquitylation; |
| | | 4,K,Ubiquitylation |
| 5477 | LRPYPKEEVGQYLKK | 1,L,Methylation; |
| | | 6,K,Ubiquitylation |
| 5482 | LRKYGKKVQTEVLQK | 1,L,Methylation; |
| | | 3,K,Ubiquitylation |
| 5498 | PFGGASHAKGIVLEK | 14,E,Methylation; |
| | | 15,K,Ubiquitylation |
| 5504 | IPLYLKGGI | 6,K,Ubiquitylation |
| 5519 | PDYDALDVANKIGI | 11,K,Ubiquitylation; |
| | | 12,I,Methylation |
| 5582 | VFHTLGQYFQKL | 11,K,Ubiquitylation |
| 5585 | LNRKGGGNL | 2,N,Deamidation; |
| | | 4,K,Ubiquitylation; |
| | | 8,N,Deamidation |
| 6316 | KYRKVLQL | 3,R,Citrullination; |
| | | 4,K,Ubiquitylation |
| 6324 | KPLPQPVF | 1,K,Ubiquitylation |
| 6376 | PRVLKQVH | 2,R,Citrullination; |
| | | 5,K,Ubiquitylation |
| 6377 | LQKKLVPFATELHER | 3,K,Ubiquitylation; |
| | | 4,K,Ubiquitylation |
| 6390 | LNRKGGGNL | 4,K,Ubiquitylation |
| 6435 | TAAAPKAGP | 6,K,Ubiquitylation |
| 6440 | EGDKYKLSKKELKEL | 1,E,Methylation; |
| | | 3,D,Methylation; |
| | | 6,K,Ubiquitylation |
| 6498 | STPTLVEVSRNLGKV | 14,K,Ubiquitylation |
| 6499 | YRFQLQATTKEGPGE | 10,K,Ubiquitylation; |
| | | 15,E,Methylation |
| 6504 | DVQHFKVLR | 6,K,Ubiquitylation |
| 6512 | DSLDYAKKNEPKHRL | 12,K,Ubiquitylation; |
| | | 14,R,Methylation |
| 6518 | AAGKRSYVL | 4,K,Ubiquitylation |
| 6535 | YLKQLLSDKQQKRQS | 12,K,Ubiquitylation |
| 6545 | KSPREPGYKAEGK | 9,K,Ubiquitylation |
| 6554 | TPLPRSWSPKDKYNY | 12,P,Oxidation; |
| | | 9,K,Ubiquitylation |
| 6567 | ASKCPKCDKTVYF | 3,K,Ubiquitylation; |
| | | 6,K,Ubiquitylation; |
| | | 1,A,Acetylation |
| 6571 | TQIFKTNTQTYRES | 5,K,Ubiquitylation |
| 6598 | TNVDKLVK | 2,N,Deamidation; |
| | | 8,K,Ubiquitylation |
| 6614 | KTNLDFKVPNG | 10,K,Acetylation; |
| | | 1,N,Deamidation; |
| | | 3,K,Ubiquitylation; |
| | | 7,N,Deamidation |
| 6633 | TVIKAPTSFGYDKPH | 4,K,Ubiquitylation |
| 6641 | TRKPPAPK | 2,R,Methylation; |
| | | 3,K,Ubiquitylation |
| 6647 | ASGGIFVLK | 9,K,Ubiquitylation |
| 6672 | VKAQYEDIAQKSK | 11,K,Ubiquitylation; |
| | | 13,K,FAT10 |
| 6677 | TEAPLNPKA | 8,K,Ubiquitylation |
| 6708 | AEITDKLGL | 6,K,Ubiquitylation |
| 6711 | VYVKEPPVF | 4,K,Ubiquitylation |
| 6717 | VVDNGSGMCK | 8,K,Ubiquitylation |
| 6723 | TATKGLIR | 4,K,Ubiquitylation |
| 6728 | TIRTKVFVW | 3,R,Methylation; |
| | | 5,K,Ubiquitylation |
| 6731 | TIDSSLKSKSL | 7,K,Dimethyl; |
| | | 9,K,Ubiquitylation |
| 6732 | TICKEANVY | 3,C,Oxidation; |
| | | 4,K,Ubiquitylation |
| 6740 | ALALPPGALAK | 11,K,Ubiquitylation |
| 6750 | ALDGGNKHFL | 7,K,Ubiquitylation |
| 6762 | TGGNFKPSQ | 6,K,Ubiquitylation |
| 6785 | NSQKDILEEKRAVP | 10,K,Ubiquitylation; |
| | | 11,R,Citrullination |
| 6828 | KEDALDFKKDKGAFY | 11,K,Ubiquitylation; |
| | | 1,E,Methylation; |
| | | 2,D,Methylation; |
| | | 3,K,Ubiquitylation; |
| | | 8,K,Ubiquitylation; |
| | | 9,K,Ubiquitylation |
| 6851 | KCHKKMGF | 4,K,Acetylation; |
| | | 5,K,Ubiquitylation |
| 6852 | KCEAAKEAL | 3,E,Methylation; |
| | | 6,K,Ubiquitylation |
| 6855 | KAVKAPGAK | 4,K,Ubiquitylation |
| 6907 | QVENQIVK | 1,Q,Deamidation; |
| | | 5,Q,Deamidation; |
| | | 8,K,Ubiquitylation |
| 6909 | QVSLKVSNDGPTLIG | 5,K,Ubiquitylation |
| 6924 | IRAAKEAKKAKQASK | 1,1,Methylation; |
| | | 5,K,Ubiquitylation |
| 6925 | LDRLAYIAHPKL | 11,K,Ubiquitylation |
| 6928 | PLGFLKVPIW | 6,K,Ubiquitylation |
| 6930 | PLVRLGLTETLGK | 13,K,Ubiquitylation |
| 6938 | IFDYDYDGLHDTEDK | 11,D,Methylation; |
| | | 15,D,Methylation; |
| | | 5,D,Methylation; |
| | | 7,K,Ubiquitylation |
| 6939 | QGPKGGSGSGPTIEE | 1,Q,Methylation; |
| | | 4,K,Ubiquitylation |
| 6946 | IKEVKEAKAKAKKES | 13,K,Ubiquitylation; |
| | | 14,K,Ubiquitylation; |
| | | 2,E,Methylation; |
| | | 5,K,Ubiquitylation; |
| | | 6,E,Methylation |
| 6951 | IIKFPLTTESAMKK | 1,I,Methylation; |
| | | 3,K,Ubiquitylation |
| 6974 | LTVTDLLGKCLLSPV | 10,K,Ubiquitylation; |
| | | 9,C,Oxidation |
| 6980 | MKHATKTAKDALSSV | 10,K,Ubiquitylation; |
| | | 9,D,Methylation |
| 6982 | MKLNISFPATGCQKL | 1,K,Ubiquitylation |
| 7015 | LSKVVNIVPVIAK | 1,L,Methylation; |
| | | 3,K,Ubiquitylation |
| 7034 | KKQQRKPLR | 5,R,Methylation; |
| | | 6,K,Ubiquitylation |
| 7083 | KGGGDILKSL | 5,D,Methylation; |
| | | 8,K,Ubiquitylation |
| 7086 | KKPKKAAGGATPK | 4,K,Dimethyl; |
| | | 5,K,Ubiquitylation |
| 7088 | LKAKKAVLKGVHSHK | L,L,Methylation; |
| | | 2,K,Ubiquitylation |
| 7098 | LKEAPEGWQTPK | 1,L,Methylation; |
| | | 2,K,Ubiquitylation |
| 7136 | SGPYGGGGQYFAKPQ | 13,K,Ubiquitylation |
| 7163 | SHEDPEVKF | 8,K,Ubiquitylation |
| 7176 | GFRTHFGGGKTTGF | 10,K,Ubiquitylation |
| 7184 | SAAKILADATAKMVE | 12,K,Ubiquitylation; |
| | | 15,E,Methylation |
| 7215 | SPKKAKAAA | 4,K,Dimethyl; |
| | | 6,K,Ubiquitylation |
| 7236 | EGKVATTVI | 3,K,Ubiquitylation |
| 7240 | SPTPQKTSAKSPGP | 10,P,Oxidation; |
| | | 12,K,Ubiquitylation; |
| | | 4,P,Oxidation |
| 7247 | SNRHGLIRKY | 9,K,Ubiquitylation |
| 7261 | SKNAVIRII | 2,K,Ubiquitylation |
| 7273 | EVEGLEANEGSKTL | 12,K,Ubiquitylation; |
| | | 14,L,Methylation |
| 7286 | KVNVFRKSRRQRK | 10,R,Citrullination; |
| | | 6,K,Ubiquitylation; |
| | | 7,R,Citrullination; |
| | | 9,R,Citrullination |
| 7299 | GHQQLYWSHPRKF | 12,K,Ubiquitylation; |
| | | 1,G,Acetylation |
| 7304 | HFELGGDKKRK | 9,K,Ubiquitylation |
| 7305 | HFDLSHGSAQVKGHG | 10,Q,Methylation; |
| | | 12,K,Ubiquitylation |
| 7310 | HGSAQVKGHGKKVAD | 12,K,Ubiquitylation; |
| | | 15,D,Methylation |
| 7312 | KYSKLLSM | 1,K,Ubiquitylation |
| 7315 | RLKGPLLNKF | 3,K,Ubiquitylation |
| 7338 | RKYVSQKK | 7,K,Ubiquitylation |
| 7339 | RKTVTAMDVVYALK | 1,R,Citrullination; |
| | | 2,K,Ubiquitylation |
| 7345 | HLVDGKSPR | 6,K,Ubiquitylation |
| 7357 | RKTGQAPGY | 2,K,Ubiquitylation |
| 7363 | RKEQKHIM | 2,K,Ubiquitylation |
| 7365 | RKKTATAV | 2,K,Ubiquitylation |
| 7367 | RKLGSHSV | 2,K,Ubiquitylation |
| 7377 | HLEDLIRK | 8,K,Ubiquitylation |
| 7379 | RTKAVGTITK | 3,K,Ubiquitylation |
| 7380 | RTKVHLPGHK | 10,L,Methylation; |
| | | 6,K,Ubiquitylation |
| 7445 | RSASPKRR | 6,K,Ubiquitylation; |
| | | 7,R,Citrullination |
| 8154 | TNVDKLVK | 8,K,Ubiquitylation |
| 8157 | KTNLDFKVPNG | 10,K,Acetylation; |
| | | 3,K,Ubiquitylation |
| 8196 | QVFNQIVK | 8,K,Ubiquitylation |
| 8279 | KKALLLYK | 2,K,Ubiquitylation |
| 8285 | NPEPKFGGKY | 2,P,Oxidation; |
| | | 4,P,Oxidation; |
| | | 5,K,Ubiquitylation |
| 8286 | SFEAQGALANIAVDK | 14,D,Methylation; |
| | | 15,K,Ubiquitylation |
| 8317 | GKRIQYQLVDISQDN | 2,K,Ubiquitylation; |
| | | 3,R,Citrullination |
| 8372 | TAYRVSKQAQLSAPT | 4,R,Citrullination; |
| | | 7,K,Ubiquitylation |
| 8381 | FSASYKTLPRGTAKE | 14,K,Ubiquitylation |
| 8385 | DVKGIKVQSVDKQYN | 12,K,Ubiquitylation; |
| | | 15,N,Deamidation |
| 8406 | DVKGIKVQSVDKQYN | 12,K,Ubiquitylation |
| 8407 | HEAVTIKCTF | 7,K,Ubiquitylation; |
| | | 8,C,Cysteinylation |
| 8408 | TKEICVVR | 2,K,Ubiquitylation |
| 8420 | TGDAYVILKTVQLRN | 9,K,Ubiquitylation |
| 8427 | HSKIIIIKKGHAKDS | 13,K,Ubiquitylation; |
| | | 14,D,Methylation |
| 8450 | STDNFNCKY | 7,C,Cysteinylation; |
| | | 8,K,Ubiquitylation |
| 8451 | STDVKGCSMY | 5,K,Ubiquitylation |
| 8481 | SERKMDPAEEDTNVY | 3,R,Citrullination; |
| | | 4,K,Ubiquitylation |
| 8487 | YPNFKDIRY | 3,N,Methylation; |
| | | 5,K,Ubiquitylation |
| 8492 | KKINNLNK | 2,K,Ubiquitylation; |
| | | 4,N,Methylation; |
| | | 5,N,Metbylation; |
| | | 7,N,Methylation |
| 8494 | YARFNKIKKLTAKDF | 3,R,Dimethyl; |
| | | 6,K,Dimethyl; |
| | | 8,K,Ubiquitylation |
| 8495 | KKFACNGTVIEH | 1,K,Ubiquitylation; |
| | | 2,K,Ubiquitylation |
| 8506 | LRPYPKEEVGQYLKK | 2,R,Methylation; |
| | | 6,K,Ubiquitylation |
| 8571 | HEAVTIKCTF | 7,K,Ubiquitylation |
| 8575 | STDNFNCKY | 8,K,Ubiquitylation |
| 8596 | KTADGKCAYR | 6,K,Ubiquitylation |
| 8604 | DTKIILETKSKTIYK | 3,K,Ubiquitylation |
| 8617 | TTKTADGKCAYR | 8,K,Ubiquitylation |
| 8624 | TYGKIWEGSSK | 4,K,Ubiquitylation |
| 8628 | TELGKLPAGGVLY | 3,L,Methylation; |
| | | 5,K,Ubiquitylation |
| 8631 | VVYVIDSCK | 8,C,Cysteinylation; |
| | | 9,K,Ubiquitylation |
| 8634 | KVFSGKSER | 1,K,Ubiquitylation |
| 8637 | KVFGGTVHKK | 9,K,Ubiquitylation |
| 8641 | VLCPPPVKK | 9,K,Ubiquitylation |
| 8642 | TKHKTILEAR | 2,K,Ubiquitylation |
| 8649 | KAFQATQQK | 1,K,Ubiquitylation |
| 8652 | PEKDIEFIYTAPSSA | 3,K,Ubiquitylation |
| 8668 | PRKVVGQQDL | 3,K,Ubiquitylation |
| 8670 | QAVLHMEQRKQQQQQ | 10,Q,Methylation; |
| | | 8,K,Ubiquitylation |
| 8690 | KHFELGGDKKRK | 11,R,Methylation; |
| | | 12,K,Ubiquitylation |
| 8693 | KGDKAFLCR | 4,K,Ubiquitylation; |
| | | 8,C,Cysteinylation |
| 8694 | KGKNIKIISKIENHE | 10,K,Ubiquitylation |
| 8704 | SKASKSSKGKD | 2,K,Sumoylation; |
| | | 8,K,Ubiquitylation |
| 8739 | RPKDYEVDATLKSLN | 12,K,Ubiquitylation; |
| | | 3,K,Oxidation |
| 8881 | VVYVIDSCK | 9,K,Ubiquitylation |
| 8885 | KGDKAFLCR | 4,K,Ubiquitylation |
| 8945 | YPFKPPKV | 4,K,Ubiquitylation |
| 8955 | SLKYPDENGFDAFLK | 3,K,Ubiquitylation |
| 8989 | ITGKPGVP | 4,K,Ubiquitylation |
| 9012 | KGEKVPKGK | 7,K,Ubiquitylation |
| 9019 | YPFKPPKV | 4,K,Ubiquitylation |
| 9067 | QKSYKVSTSGPRAFS | 2,K,Ubiquitylation; |
| | | 5,K,Sumoylation |
| 9069 | GKVTKSAQKAQKAK | 2,K,Ubiquitylation |
| 9070 | FINIPVLDIK | 10,N,Deamidation; |
| | | 3,K,Ubiquitylation |
| 9071 | FINIPVLDIK | 3,K,Ubiquitylation |
| 9082 | KPEPPAMPQPVPTA | 1,K,Ubiquitylation |
| 9093 | FPDKPITQY | 4,K,Ubiquitylation |
| 9114 | TKGGDAPAAGEDA | 2,K,Ubiquitylation |
| 9126 | TPKIQVYSRHPAENG | 3,K,Ubiquitylation; |
| | | 4,I,Methylation |
| 9147 | VHKAVLTIDEKGTEA | 11,K,Ubiquitylation; |
| | | 14,E,Methylation |
| 9170 | QGQKKVEELEGEITT | 1,Q,Methylation; |
| | | 4,K,Ubiquitylation |
| 9192 | KVFSGKSER | 1,K,Ubiquitylation |
| 9202 | LANIAVDKANLEIMT | 7,D,Methylation; |
| | | 8,K,Ubiquitylation |
| 9222 | SNLRKAFEEAEKNAP | 12,K,Ubiquitylation; |
| | | 13,N,Methylation |
| 9252 | ALADAKALV | 4,D,Methylation; |
| | | 6,K,Ubiquitylation |
| 9256 | EEIAFLKKL | 8,K,Ubiquitylation |
| 9319 | VDRYISKYELDKAFS | 7,K,Ubiquitylation |
| 9331 | PKVLANHLL | 2,K,Ubiquitylation |
| 9366 | LYAEKVATR | 5,K,Ubiquitylation; |
| | | 9,R,Citrullination |
| 9368 | TNKVASQKGMSVY | 3,K,Ubiquitylation |
| 9378 | AVHKAVLTIDEKGTE | 11,E,Methylation; |
| | | 12,K,Ubiquitylation; |
| | | 15,E,Methylation |
| 9438 | SQKPVMVKR | 8,K,Ubiquitylation |
| 9447 | KPLATKAAR | I,K,Ubiquitylation; |
| | | 2,P,Oxidation |
| 9454 | EAVYCKFHYK | 5,C,Cysteinylation; |
| | | 6,K,Ubiquitylation |
| 9455 | EAVYCKFHYK | 6,K,Ubiquitylation |
| 9465 | VDLLKLSV | 2,D,Methylation; |
| | | 5,K,Ubiquitylation |
| 9472 | RPKDYEVDATLKSLN | 12,K,Ubiquitylation |
| 9479 | VHKAVLTIDEKGTEA | 11,K,Ubiquitylation; |
| | | 14,E,Methylation |
| 9506 | KABAKAKAL | 3,E,Methylation; |
| | | 5,K,Ubiquitylation |
| 9512 | KINLLKRSL | 3,N,Deamidation; |
| | | 6,K,Ubiquitylation |
| 9514 | KINLLKRSL | 6,K,Ubiquitylation |
| 9520 | KESTLHLVL | 1,K,Ubiquitylation |
| 9555 | FIDLLHDK | 6,H,Methylation; |
| | | 8,K,Ubiquitylation |
| 9560 | AQLGGPEAAKSDETA | 10,K,Ubiquitylation; |
| | | 12,D,Methylation |
| 9569 | KRTKKVGIVGKY | 1,K,Ubiquitylation; |
| | | 2,R,Methylation |
| 9589 | TKGGDAPAAGEDA | 2,K,Ubiquitylation |
| 9608 | TPKIQVYSRHPAEN | 3,K,Ubiquitylation; |
| | | 4,1,Methylation |
| 9643 | AGKVTKSAQKAQKAK | 3,K,Ubiquitylation |
| 9650 | REAKKQGP | 5,K,Ubiquitylation |
| 9740 | FLLARKATIQK | 2,L,Methylation; |
| | | 6,K,Ubiquitylation |
| 9776 | VPPVQVSPLIKL | 11,K,Ubiquitylation |
| 9787 | GHQQLYWSHPRKF | 12,K,Ubiquitylation |
| 9792 | KVKVGVNGFG | 1,K,Ubiquitylation |
| 9798 | SILSLVTKI | 8,K,Ubiquitylation |
| 9799 | KVPKLLIY | 4,K,Ubiquitylation |
| 9804 | VETRPAGDGTFQKWA | 12,Q,Methylation; |
| | | 13,K,Ubiquitylation |
| 9836 | NKNISAIIQGIGKDK | 2,K,Ubiquitylation |
| 9894 | EESEKLSKMSSLLE | 10,K,Ubiquitylation; |
| | | 5,S,Phosphorylation; |
| | | 7,K,Ubiquitylation; |
| | | 8,S,Phosphorylation |
| 9952 | TKDVPITSV | 2,K,Ubiquitylation |
| 9995 | FVKEFSHIAFLTIKG | 13,I,Methylation; |
| | | 14,K,Ubiquitylation |
| 10022 | KGQKYFDSGDYNMAK | 1,K,Methylation; |
| | | 4,K,Ubiquitylation |
| 10033 | DKPDMAEIEKFDKSK | 1,D,Methylation; |
| | | 2,K,Ubiquitylation |
| 10038 | VLCPPPVKKR | 9,K,Ubiquitylation |
| 10061 | VLCPPPVKKR | 8,K,Ubiquitylation; |
| | | 9,K,Ubiquitylation |
| 10077 | AVYLSTCKDSK | 8,K,Ubiquitylation |
| 10083 | TGKTLIGK | 3,K,Ubiquitylation |
| 10096 | KKILKVMKK | 2,K,Ubiquitylation; |
| | | 4,L,Methylation; |
| | | 5,K,Ubiquitylation |
| 10106 | HVSGGLLK | 8,K,Ubiquitylation |
| 10157 | KGPPKALAYK | 5,K,Ubiquitylation |
| 10182 | HPKYKTEL | 3,K,Ubiquitylation |
| 10214 | SQVMREWEEAERQAK | 15,K,Ubiquitylation |
| 10235 | HKAVLTIDEKGTEAA | 10,K,Ubiquitylation |
| 10355 | HTDILKEKY | 8,K,Ubiquitylation |
| 10424 | TDKTPALISDY | 3,K,Ubiquitylation |
| 10433 | KRKIVLDPSGSMN | 2,R,Methylation; |
| | | 3,K,Ubiquitylation |
| 10509 | TDQQKLIY | 3,Q,Deamidation; |
| | | 4,Q,Deamidation; |
| | | 5,K,Ubiquitylation |
| 10547 | TDQQKLIY | 5,K,Ubiquitylation |
| 10574 | GSSSPLRK | 8,K,Ubiquitylation |
| 10617 | KTDGKKSY | 5,K,Ubiquitylation |
| 10633 | QHEKKYDI | 4,K,Ubiquitylation; |
| | | 5,K,Acetylation |
| 10637 | TKLPNSVLGR | 2,K,Ubiquitylation |
| 10646 | HTDILKEKY | 8,K,Ubiquitylation |
| 10826 | AK(GG)AETIQAL | 2,K,Ubiquitylation |

The agents of some embodiments of the invention are capable of specifically binding the peptide when is presented by (or bound to) an MHC molecule.

As used herein, the phrase “major histocompatibility complex (MHC)” refers to a complex of antigens encoded by a group of linked loci that plays a role in control of the cellular interactions responsible for physiologic immune responses, which are collectively termed H-2 in the mouse and “human leukocyte antigen (HLA)” in humans. The two principal classes of the MHC antigens, class I and class II, each comprise a set of cell surface glycoproteins which play a role in determining tissue type and transplant compatibility.

According to a specific embodiment, the MHC is a human MHC (i.e. HLA).

According to a specific embodiment, the MHC is a MHC class I.

According to a specific embodiment, the MHC is HLA class I.

MHC class I molecules are expressed on the surface of nearly all cells. These molecules function in presenting peptides which are mainly derived from endogenously synthesized proteins to CD8+ T cells via an interaction with the αβ T-cell receptor. The class I MHC molecule is a heterodimer composed of a 46-kDa heavy chain which is non-covalently associated with the 12-kDa light chain β-2 microglobulin. In humans, there are several MHC haplotypes, such as, for example, HLA-A2, HLA-A1, HLA-A3. HLA-A24, HLA-A26, HLA-A28, HLA-A31, HLA-A33, HLA-A34, HLA-A0201, HLA-A6802, HLA-A3101, HLA-B7, HLA-B27, HLA-B45, HLA-B5401, HLA-B5101, HLA-B4402, HLA-B4403 and HLA-Cw8, their sequences can be found for example at the kabbat data base, at htexttransferprotocol://immuno.bme.nwu.edu. Further information concerning MHC haplotypes can be found in Paul, B. Fundamental Immunology Lippincott-Rven Press.

According to specific embodiments, the MHC haplotype comprises a haplotype selected from the group consisting of HLA-A0201, HLA-B5401, HLA-B5101, HLA-A6802. HLA-B4402, HLA-B4403 and HLA-A3101.

According to other specific embodiments, the MHC is a MHC class II.

According to a specific embodiment, the MHC is HLA class II. According to specific embodiments, the agent binds the modified or the un-modified peptide in an MHC-restricted manner (i.e. does not bind the MHC in an absence of the peptide, and does not bind the peptide in an absence of the MHC).

According to a specific embodiment, the agent is capable of binding the MHC presented modified or un-modified peptide when naturally presented on cells.

As used herein, the term “specifically binding an MHC presented peptide comprising a PTM” refers to the ability to bind the modified peptide and not a peptide having the same amino acid sequence as said peptide that does not comprise the modification, which may be manifested as higher affinity (e.g., K_{d}) to the modified peptide as compared to the non-modified peptide.

According to specific embodiments, the agent is capable of binding the modified peptide and not a peptide having a different amino acid sequence or a peptide having a different modification, which may be manifested as higher affinity (e.g., K_{d}) to the modified peptide as compared to other peptides.

As used herein, the term “specifically binding an MHC presented peptide” refers to the ability to bind the peptide and not a peptide having a different amino acid sequence, which may be manifested as higher affinity (e.g., K_{d}) to the peptide as compared to other peptides.

Higher affinity can be, for examples, of at least 5, 10, 100, 1000 or 10000 fold.

Methods of determining binding of the agent to the peptide are well known in the art and include BiaCore, HPLC, Surface Plasmon Resonance assay (SPR) and flow cytometry.

According to specific embodiments, the agent binds the MHC presented peptide with an affinity higher than 10⁻⁶ M.

According to specific embodiments, the agent binds the MHC presented peptide with an affinity higher than about, 10⁻⁹ M, 10⁻¹⁰ M and as such is stable under physiological (e.g., in vivo) conditions.

According to a specific embodiment the affinity is between 0.1-10⁻⁹ M or 1-10×10⁻⁹ M or 0.1-10×10⁻⁹ M. According to specific embodiments affinity is of at least 100 nM, 50 nM, 10 nM, 1 nM or higher.

Non-limiting examples of agents capable of binding the MHC presented modified or un-modified peptides include, but are not limited to, antibodies, immune cells e.g. T cells NK cells, CAR-T cells, CAR-NK cells, PROTACS, small molecules, chemicals, toxins and drugs.

Thus, according to specific embodiments, the agent is an antibody.

The term “antibody” as used in this invention includes intact molecules as well as functional fragments thereof (such as Fab. F(ab′)2, Fv, scFv, dsFv, or single domain molecules such as VH and VL) that are capable of binding to an epitope of an antigen. According to specific embodiments, the antibodies of some embodiments of the present invention bind the peptide in an MHC restricted manner. These antibodies are referred to as T cell receptor like antibodies.

According to specific embodiments, the antibody is a whole or intact antibody.

According to specific embodiments, the antibody is an antibody fragment.

According to specific embodiments, the antibody comprises an Fc domain.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992. Sequences of Proteins of Immunological Interest, 5th ed., Public Health Service. NIH. Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989. Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and
(vii) Single domain antibodies or nanobodies are composed of a single VH or VL domains which exhibit sufficient affinity to the antigen.

According to specific embodiments the antibody heavy chain constant region is chosen from, e.g., IgG1, IgG2, IgG3, IgG4, IgM, IgA1, IgA2, IgD, and IgE.

According to a specific embodiment the antibody isotype is IgG1 or IgG4.

The choice of antibody type will depend on the immune effector function that the antibody is designed to elicit.

The antibody may be monoclonal or polyclonal.

Methods of producing polyclonal and monoclonal antibodies as well as fragments thereof are well known in the art (See for example, Harlow and Lane. Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg. U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al, and Boerner et al, are also available for the preparation of human monoclonal antibodies (Cole et al., Monoclonal Antibodies and Cancer Therapy. Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Once antibodies are obtained, they may be tested for activity, for example via ELISA.

The antibody may be soluble or non-soluble.

Non-soluble antibodies may be a part of a particle (synthetic or non-synthetic) or a cell.

According to other specific embodiments, the agent is a T cell receptor (TCR) or a chimeric antigen receptor (CAR).

As used herein the phrase “T cell receptor (TCR)” refers to variable α- and β-chains from T cells with specificity against a specific peptide presented in the context of MHC.

According to specific embodiments, the agent is not a naturally occurring TCR.

As used herein the phrase “chimeric antigen receptor (CAR)” refers to a recombinant or synthetic molecule which combines antibody-based specificity for a desired peptide with a T cell receptor-activating intracellular domain to generate a chimeric protein that exhibits cellular immune activity to the specific antigen.

According to other specific embodiments, the agent comprises a therapeutic moiety.

The therapeutic moiety can be proteinaceous or non-proteinaceous.

The Therapeutic moiety may be any molecule, including small molecule chemical compounds and polypeptides.

According to specific embodiments, the therapeutic moiety is capable of eliciting an immune response to a cell presenting the peptide upon binding of the agent.

As used herein, the phrase “eliciting an immune response” refers to stimulation of an immune cell (e.g. T cell, dendritic cell, NK cell, B cell) that results in cellular proliferation, maturation, cytokine production and/or induction of regulatory or effector functions.

According to specific embodiments, the immune response comprises a T cell response.

According to specific embodiments, the immune response comprises a dendritic cell response.

According to specific embodiments, the immune response is specific to a cell expressing the modified peptide with no cross reactivity with a cell not expressing the modified peptide.

According to specific embodiments, the immune response is specific to a cell expressing the un-modified peptide with no cross reactivity with a cell not expressing the un-modified peptide.

Methods of evaluating immune cell activation or function are well known in the art and include, but are not limited to, proliferation assays such as BRDU and thymidine incorporation, cytotoxicity assays such as chromium release, cytokine secretion assays such as intracellular cytokine staining ELISPOT and ELISA, expression of activation markers such as CD25, CD69 and CD69 using flow cytometry and multimer (e.g. tetramer) assays.

The therapeutic moiety can be an integral part of the agent e.g., in the case of a whole antibody, the Fc domain, which activates antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. Classical ADCC is mediated by natural killer (NK) cells; macrophages, neutrophils and eosinophils can also mediate ADCC. For example, eosinophils can kill certain parasitic worms known as helminths through ADCC mediated by IgE. ADCC is part of the adaptive immune response due to its dependence on a prior antibody response.

Alternatively or additionally, the agent may be a bispecific antibody (see e.g., Withoff, S., Helfrich. W., de Leij, L F., Molema, G. (2001) Curr Opin Mol Tier. 3,:53-62) in which the therapeutic moiety is a T cell engager for example, such as an anti CD3 antibody or an anti CD16a; alternatively the therapeutic moiety may be an anti-immune checkpoint molecule (anti PD-1).

Alternatively or additionally, according to specific embodiments, the therapeutic moiety is an immune cell expressing the agent. Non-limiting examples of immune cells that can be used with specific embodiments of the invention include T cells. NK cells. NKT cells. B cells, macrophages, dendritic cells (DCs) and granulocytes.

According to specific embodiments, the immune cell is a T cell.

Thus, according to specific embodiments, the agent is a T cell receptor (TCR) or a chimeric antigen receptor (CAR) and the therapeutic moiety is a T cell transduced with the agent.

Method of transducing with a TCR are known in the art and are disclosed e.g. in Nicholson et al. Adv Hematol. 2012; 2012:404081; Wang and Rivière Cancer Gene Ther. 2015 March; 22(2):85-94); and Lamers et al. Cancer Gene Therapy (2002) 9, 613-623.

Method of transducing with a CAR are known in the art and are disclosed e.g. in Davila et al. Oncoimmunology. 2012 Dec. 1; 1(9):1577-1583; Wang and Rivière Cancer Gene Ther. 2015 March; 22(2):85-94); and Maus et al. Blood. 2014 Apr. 24; 123(17):2625-35.

Alternatively or additionally the agent may be attached to a heterologous therapeutic moiety (methods of conjugation are described hereinbelow). The therapeutic moiety can be, for example, a cytotoxic moiety, a toxic moiety [e.g., Pseudomonas exotoxin (GenBank Accession Nos. AAB25018 and S53109); PE38KDEL; Diphtheria toxin (GenBank Accession Nos. E00489 and E00489); Ricin A toxin (GenBank Accession Nos. 225988 and A23903)], a cytokine moiety [e.g., interleukin 2 (GenBank Accession Nos. CAA00227 and A02159), interleukin 10 (GenBank Accession Nos. P22301 and M57627)], a drug, a chemical, a protein and/or a radioisotope.

According to specific embodiments, the therapeutic moiety is selected from the group consisting of a toxin, a drug, a chemical, a protein and a radioisotope.

According to some embodiments of the invention, the therapeutic moiety is conjugated by translationally fusing the polynucleotide encoding the agent of some embodiments of the invention with the nucleic acid sequence encoding the therapeutic moiety.

Additionally or alternatively, the therapeutic moiety can be chemically conjugated (coupled) to the agent of the invention, using any conjugation method known to one skilled in the art. For example, a peptide can be conjugated to an agent of interest, using a 3-(2-pyridyldithio)propionic acid Nhydroxysuccinimide ester (also called N-succinimidyl 3-(2-pyridyldithio) propionate) (“SDPD”) (Sigma, Cat. No. P-3415; see e.g., Cumber et al. 1985, Methods of Enzymology 112: 207-224), a glutaraldehyde conjugation procedure (see e.g., G. T. Hermanson 1996, “Antibody Modification and Conjugation, in Bioconjugate Techniques. Academic Press, San Diego) or a carbodiimide conjugation procedure [see e.g., J. March. Advanced Organic Chemistry: Reaction's, Mechanism, and Structure, pp. 349-50 & 372-74 (3d ed.), 1985; B. Neises et al. 1978, Angew Chem., Int. Ed. Engl. 17:522; A. Hassner et al. 1978, Tetrahedron Lett. 4475; E. P. Boden et al. 1986. J. Org. Chem. 50:2394 and L. J. Mathias 1979. Synthesis 561].

According to specific embodiments the agent is bound to a detectable moiety.

Examples of detectable moieties that can be used in the present invention include but are not limited to radioactive isotopes, phosphorescent chemicals, chemiluminescent chemicals, fluorescent chemicals, enzymes, fluorescent polypeptides, a radioactive isotope (such as ^{[125]}iodine) and epitope tags. The detectable moiety can be a member of a binding pair, which is identifiable via its interaction with an additional member of the binding pair, and a label which is directly visualized. In one example, the member of the binding pair is an antigen which is identified by a corresponding labeled antibody. In one example, the label is a fluorescent protein or an enzyme producing a colorimetric reaction.

Further examples of detectable moieties, include those detectable by Positron Emission Tomagraphy (PET) and Magnetic Resonance Imaging (MRI), all of which are well known to those of skill in the art.

Any of the proteinaceous agents described herein can be encoded from a polynucleotide. These polynucleotides can be used as therapeutics per se or in the recombinant production of the agent or the peptide.

Thus, according to an aspect of the present invention there is provided a polynucleotide encoding the agent or the peptide.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

To express exogenous peptide or agent in mammalian cells, a polynucleotide sequence encoding the agent is preferably ligated into a nucleic acid construct suitable for mammalian cell expression.

Thus, according to an aspect of the present invention there is provided a nucleic acid construct comprising the isolated polynucleotide.

Such a nucleic acid construct or system includes at least one cis-acting regulatory element for directing expression of the nucleic acid sequence. Cis-acting regulatory sequences include those that direct constitutive expression of a nucleotide sequence as well as those that direct inducible expression of the nucleotide sequence only under certain conditions. Thus, for example, a promoter sequence for directing transcription of the polynucleotide sequence in the cell in a constitutive or inducible manner is included in the nucleic acid construct.

Also provided are cells which comprise the polynucleotides/expression vectors as described herein.

Such cells are typically selected for high expression of recombinant proteins (e.g., bacterial, plant or eukaryotic cells e.g., CHO. HEK-293 cells), but may also be an immune cell (e.g., macrophages, dendritic cells. T cells. B cells or NK cells) when for instance the CDRs of the agent are implanted in a T Cell Receptor or CAR transduced in said cells which are used in adoptive cell therapy.

The expression pattern of the peptides described herein renders the agents that bind them particularly suitable for diagnostic and therapeutic applications.

Thus, according to an aspect of the present invention there is provided a method of eliciting an immune response in a subject in need thereof, the method comprising administering to the subject an effective amount of the agent or an immune cell expressing same, thereby eliciting an immune response in the subject.

As used herein, the term “subject” refers to humans and animals having an MHC system, such as the HLA system in humans. The subject may be of any gender and of any age.

According to specific embodiments, the subject is a human subject.

According to specific embodiments, the subject expresses HLA class I haplotype selected from the group consisting of HLA-A0201. HLA-B5401, HLA-B5101. HLA-A6802, HLA-B4402. HLA-B4403 and HLA-A3101.

According to specific embodiments, the subject is diagnosed with a disease (i.e., cancer) or is at risk of to develop a disease (i.e. cancer).

According to other specific embodiments, the subject is not diagnosed with cancer and is undergoing a routine well-being checkup.

According to specific embodiments, the subject is at risk of having cancer (e.g., a genetically predisposed subject, a subject with medical and/or family history of cancer, a subject who has been exposed to carcinogens, occupational hazard, environmental hazard) and/or exhibits suspicious clinical signs of cancer [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness].

According to specific embodiments, cells of the subject present the peptide at a level above a predetermined threshold.

According to an additional or an alternative aspect of the present invention, there is provided a method of treating cancer in a subject in need thereof, the method comprising administering to the subject a therapeutically effective amount of the agent or the cell expressing same, thereby treating the cancer in the subject.

According to an additional or an alternative aspect of the present invention, there is provided the agent or the cell expressing same, for use in treating cancer in a subject in need thereof.

As used herein the term “treating” refers to inhibiting, preventing or arresting the development of a pathology (disease, disorder, or condition e.g., cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

According to specific embodiments, treatment may be evaluated by a decrease in tumor volume, a decrease in the number of tumor cells, a decrease in the number of metastases, an increase in life expectancy, or amelioration of various physiological symptoms associated with the cancerous condition.

As used herein, the term cancer encompasses both malignant and pre-malignant cancers.

According to specific embodiments, the cancer comprises malignant cancer.

Cancers which can be treated by the methods of some embodiments of the invention can be any solid or non-solid cancer and/or cancer metastasis. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma. Hodgkin lymphoma, chronic lymphocytic leukemia (CLL); acute lymphoblastic leukemia (ALL); Acute myeloid leukemia (AML). Acute promyelocytic leukemia (APL). Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma. Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias. Examples of small or microscopic pre-malignant cancers include HGSIL (High grade squamous intraepithelial lesion of uterine cervix). AIN (anal intraepithelial neoplasia), dysplasia of vocal cord, aberrant crypts (of colon). PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome. C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is selected from the group consisting of glioblastoma, B cell leukemia, meningioma, melanoma, colon cancer and breast cancer.

According to specific embodiments, cancerous cells present the disclosed peptide.

According to specific embodiments, when the modified peptide amino acid sequence is selected from the group consisting of SEQ ID NO: 1-209 and 10819; said cancer is B cell leukemia.

According to specific embodiments, when the modified peptide amino acid sequence is selected from the group consisting of SEQ ID NO: 210-943; the cancer is breast cancer.

According to specific embodiments, when the modified peptide amino acid sequence is selected from the group consisting of SEQ ID NO: 944-1117 and 10820; the cancer is colon cancer.

According to specific embodiments, when the modified peptide amino acid sequence is selected from the group consisting of SEQ ID NO: 1118-1691 and 10817; the cancer is glioblastoma.

According to specific embodiments, when the modified peptide amino acid sequence is selected from the group consisting of SEQ ID NO: 1962-8276; the cancer is melanoma.

According to specific embodiments, when the modified peptide amino acid sequence is selected from the group consisting of SEQ ID NO: 8277-8897; the cancer is meningioma.

According to specific embodiments, when the un-modified peptide amino acid sequence is selected from the group consisting of SEQ ID NO: 10747-10748; the cancer is B cell leukemia.

According to specific embodiments, when the un-modified peptide amino acid sequence is selected from the group consisting of SEQ ID NO: 10749-10756 and 10822; the cancer is breast cancer.

According to specific embodiments, when the un-modified peptide is as set forth in SEQ ID NO: 10757; the cancer is colon cancer.

According to specific embodiments, when the un-modified peptide amino acid sequence is selected from the group consisting of SEQ ID NO: 10758-10796; the cancer is melanoma.

According to specific embodiments, when the un-modified peptide amino acid sequence is selected from the group consisting of SEQ ID NO: 10797-10806; the cancer is meningioma.

According to specific embodiments, cells of the cancer present the peptide at a level above a predetermined threshold.

Such a predetermined threshold can be experimentally determined by comparing presentation levels in a biological sample derived from subjects diagnosed with cancer to a biological sample obtained from healthy subjects (e.g., not having cancer). Alternatively or additionally, such a predetermined threshold can be experimentally determined by comparing presentation levels in cancer cells to presentation levels in healthy cells obtained from the same subject. Alternatively, such a level can be obtained from the scientific literature and from databases.

According to specific embodiments, the level above a predetermined threshold is statistically significant.

According to specific embodiments the increase from a predetermined threshold is at least 5%, at least 10%, at least 20%, at least 30%, at least 40%, at least 50%, at least 60%, at least 70%, at least 80%, at least 90%, at least 95%, at least 100% or more, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more as compared to the control sample as measured using the same assay.

Methods of determining presentation of the peptides are known in the art, and include e.g. flow cytometry, immunohistochemistry and the like.

Alternatively or additionally, the expression pattern of the peptides described herein renders them suitable for therapeutic applications e.g, as anti-cancer vaccines.

Thus, according to an aspect of the present invention there is provided a method of eliciting an immune response in a subject in need thereof, the method comprising administering to the subject an effective amount of a peptide comprising an amino acid sequence selected from the group consisting of SEQ ID NO: 1-10746, 10817, 10819, 10820, 10823, 10824, 10826 and 10827 having the corresponding modification according to Table 3, thereby eliciting an immune response to a cell presenting said amino acid sequence having said corresponding modification in the subject.

Alternatively or additionally, according to an aspect of the present invention there is provided a method of eliciting an immune response in a subject in need thereof, the method comprising administering to the subject an effective amount of a peptide comprising an amino acid sequence having a ubiquitin or a ubiquitin-like (UBL) modifier tail, thereby eliciting an immune response to a cell presenting said amino acid sequence having said ubiquitin or said UBL modifier tail in the subject.

Alternatively or additionally, according to an aspect of the present invention there is provided a method of eliciting an immune response in a subject in need thereof, the method comprising administering to the subject an effective amount of a peptide comprising an amino acid sequence selected from the group consisting of SEQ ID NO: 10747-10816 and 10822, thereby eliciting an immune response to a cell presenting said amino acid sequence in the subject.

Alternatively or additionally, according to an aspect of the present invention there is provided a method of treating cancer in a subject in need thereof, the method comprising administering to the subject a therapeutically effective amount of a peptide comprising an amino acid sequence selected from the group consisting of SEQ ID NO: 1-10746, 10817, 10819, 10820, 10823, 10824, 10826 and 10827 having the corresponding modification according to Table 3, thereby treating the cancer in the subject.

Alternatively or additionally, according to an aspect of the present invention there is provided a method of treating cancer in a subject in need thereof, the method comprising administering to the subject a therapeutically effective amount of a peptide comprising an amino acid sequence having a ubiquitin or a ubiquitin-like (UBL) modifier tail, thereby treating the cancer in the subject.

Alternatively or additionally, according to an aspect of the present invention there is provided a method of treating cancer in a subject in need thereof, the method comprising administering to the subject a therapeutically effective amount of a peptide comprising an amino acid sequence selected from the group consisting of SEQ ID NO: 10747-10816 and 10822, thereby treating the cancer in the subject.

Alternatively or additionally, according to an aspect of the present invention there is provided a peptide comprising an amino acid sequence selected from the group consisting of SEQ ID NO: 1-10746, 10817, 10819, 10820, 10823, 10824, 10826 and 10827 having the corresponding modification according to Table 3, for use in treating cancer in a subject in need thereof.

Alternatively or additionally, according to an aspect of the present invention there is provided a peptide comprising an amino acid sequence having a ubiquitin or a ubiquitin-like (UBL) modifier tail, for use in treating cancer in a subject in need thereof.

Alternatively or additionally, according to an aspect of the present invention there is provided a peptide comprising an amino acid sequence selected from the group consisting of SEQ ID NO: 10747-10816 and 10822, for use in treating cancer in a subject in need thereof.

According to specific embodiments, the amino acid sequence having a ubiquitin or a ubiquitin-like (UBL) modifier tail is selected from the group of sequences listed in Table 5.

According to specific embodiments, the peptide is capable of being presented by a MHC molecule.

According to specific embodiments, the peptide is capable of eliciting an immune response to a cell presenting the specified amino acid sequence.

According to specific embodiments, the peptide is capable of eliciting an immune response to a cell presenting the specified amino acid sequence having the corresponding modification or the ubiquitin or UBL modifier tail.

Methods of determining the ability to elicit an immune response are known in the art and are further described hereinabove.

According to specific embodiments, the peptide is no more than 50 amino acids in length.

According to specific embodiments, the peptide is between 9-50 amino acids, 9-40 amino acids, 9-30 amino acids, 9-20 amino acids, or between 9-13 amino acids long.

According to specific embodiments, the peptide is no more than 20 amino acids in length.

According to specific embodiments, the peptide is no more than 14 amino acids in length.

According to specific embodiments, the peptide amino acid sequence consists of the amino acid sequence specified.

The term “peptide” in the aspects referring to their use encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” in the aspects referring to their use is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 6 and 7 below list naturally occurring amino acids (Table 6), and non-conventional or modified amino acids (e.g., synthetic, Table 7) which can be used with some embodiments of the invention.

TABLE 6

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 7

| Non-conventional amino | | | |
|---|---|---|---|
| acid | Code | Non-conventional amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl)glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| C-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclobexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides or proteinaceous agents of some embodiments of the invention may be synthesized by any techniques that are known to those skilled in the art of peptide synthesis, including, but not limited to solid phase and recombinant techniques. For solid phase peptide synthesis, a summary of the many techniques may be found in J. M. Stewart and J. D. Young. Solid Phase Peptide Synthesis, W. H. Freeman Co. (San Francisco), 1963 and J. Meicnhofer, Hormonal Proteins and Peptides, vol. 2, p. 46, Academic Press (New York), 1973. For classical solution synthesis see G. Schroder and K. Lupke. The Peptides, vol. 1. Academic Press (New York), 1965. A detailed description on recombinant production is provided hereinabove.

The N and C termini of the peptides and proteinaceous agents of some embodiments of the present invention may be protected by function groups. According to specific embodiments, the function group does not compromise the biological activity (e.g. being presented by a MHC molecule; eliciting an immune response to a cell presenting the amino acid sequence specified) of the peptide or agent. Suitable functional groups are described in Green and Wuts. “Protecting Groups in Organic Synthesis”. John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O— CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom. R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃. —N(CH₃)₂, —NH(ethyl), —N(ethyl)₂, —N(methyl) (ethyl), —NH(benzyl), —N(C1-C4 alkyl)(benzyl). —NH(phenyl), —N(C1-C4 alkyl) (phenyl), —OCH₃, —O-(ethyl), —O-n-propyl), —O-(n-butyl), —O-(iso-propyl), —O-(sec-butyl), —O-(t-butyl), —O-benzyl and —O-phenyl.

The present invention further provides peptide conjugates and fusion polypeptides comprising the peptides disclosed herein.

The peptides of some embodiments of the present invention may be used alone or in combination (e.g., other peptide as disclosed herein or with other heterologous moieties e.g., Ig domain). Thus, the peptides may be used in a mixture and/or as a chimeric peptide with one or more additional peptides. As used herein, the term “mixture” is defined as a non-covalent combination of peptides existing in variable proportions to one another, whereas the term “chimeric peptide” is defined as at least two identical or non-identical peptides covalently attached one to the other. Such attachment can be any suitable chemical linkage, direct or indirect, as via a peptide bond, or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched or cyclic side chains, internal carbon or nitrogen atoms, and the like.

Thus, according to an aspect of the present invention there is provided a multimer of the peptides disclosed herein. The multimer may be a homo- or a hetero-multimer.

According to another aspect of the present invention there is provided a fusion protein comprising at least one of peptides disclosed herein.

According to specific embodiments the peptide is complexed with a MHC molecule, such e.g., as disclosed in U.S. Pat. Nos. 7,399,838 and 5,734,023, US Application Publication no. US20050003431 and International Application Publication no. WO2009039854A2.

The peptides and agents of some embodiments may be attached (either covalently or non-covalently) to a penetrating agent.

As used herein the phrase “penetrating agent” refers to an agent which enhances translocation of any of the attached peptide or agents across a cell membrane.

According to one embodiment, the penetrating agent is a peptide and is attached to the peptide or proteinaceous agent (either directly or non-directly) via a peptide bond.

Typically, peptide penetrating agents have an amino acid composition containing either a high relative abundance of positively charged amino acids such as lysine or arginine, or have sequences that contain an alternating pattern of polar/charged amino acids and non-polar, hydrophobic amino acids.

According to specific embodiments, the peptide or agent is provided in a formulation suitable for cell penetration that enhances intracellular delivery of the polypeptide or agent as further described hereinbelow.

By way of non-limiting example, cell penetrating peptide (CPP) sequences may be used in order to enhance intracellular penetration; however, the disclosure is not so limited, and any suitable penetrating agent may be used, as known by those of skill in the art.

Cell-Penetrating Peptides (CPPs) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. Therefore, according to additional exemplary embodiment CPPs can be used to transport the polypeptide or the composition of matter to the interior of cells. TAT (transcription activator from HIV-1), pAntp (also named penetratin, Drosophila antennapedia homeodomain transcription factor) and VP22 (from Herpes Simplex virus) are examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15(11): 7158-7167]. Fawell S. et al. [Proc Natl Acad Sci USA. (1994) 91:664-668], and Jing Bian et al. [Circulation Research (2007) 100: 1626-1633].

According to other specific embodiments of the invention, the peptide or proteinaceous agent is attached to non-amino acid moieties, such as for example, hydrophobic moieties (various linear, branched, cyclic, polycyclic or hetrocyclic hydrocarbons and hydrocarbon derivatives) attached to the peptides; non-peptide penetrating agents; various protecting groups, especially where the compound is linear, which are attached to the compound's terminals to decrease degradation. Chemical (non-amino acid) groups present in the compound may be included in order to improve various physiological properties such as: improve uptake into cells (e.g. cancer cells); decreased degradation or clearance; decreased repulsion by various cellular pumps, improve immunogenic activities, improve various modes of administration; increased specificity, increased affinity, decreased toxicity and the like.

According to specific embodiments, the peptide or proteinaceous agent and the attached non-proteinaceous moiety are covalently or non-covalently attached, directly or through a spacer or a linker. Modes of binding are described hereinabove and below.

Attaching the amino acid sequence component of the peptides or proteinaceous agent to other non-amino acid agents may be by covalent linking, by non-covalent complexion, for example, by complexion to a hydrophobic polymer, which can be degraded or cleaved producing a compound capable of sustained release; by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

Exemplary non-proteinaceous moieties which may be used with specific embodiments of the invention include, but are not limited to a drug, a chemical, a small molecule, a polynucleotide, a detectable moiety, polyethylene glycol (PEG), Polyvinyl pyrrolidone (PVP), poly(styrene comaleic anhydride) (SMA), and divinyl ether and maleic anhydride copolymer (DIVEMA). According to specific embodiments, the non-proteinaceous moiety comprises polyethylene glycol (PEG).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

Bioconjugation of the peptide amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG. PEG p-nitrophenyl carbonates (PEG-NPC, such as methoxy PEG-NPC), PEG aldehydes. PEG-orthopyridyl-disulfide, carbonyldimidazol-activated PEGs, PEG-thiol, PEG-maleimide. Such PEG derivatives are commercially available at various molecular weights [See, e.g., Catalog. Polyethylene Glycol and Derivatives, 2000 (Shearwater Polymers. Inc., Huntsvlle, Ala.)]. If desired, many of the above derivatives are available in a monofunctional monomethoxyPEG (mPEG) form. In general, the PEG added to the peptide of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson. Bioconjugate Techniques, Academic Press San Diego. Calif. (1996), at Chapter 15 and in Zalipsky et al., “Succinimidyl Carbonates of Polyethylene Glycol.” in Dunn and Ottenbrite, eds., Polymeric Drugs and Drug Delivery Systems, American Chemical Society, Washington, D.C. (1991).

Conveniently, PEG can be attached to a chosen position in the peptide or proteinaceous agent by site-specific mutagenesis as long as the activity of the conjugate is retained. A target for PEGylation could be any Cysteine residue at the N-terminus or the C-terminus of the peptide sequence. Additionally or alternatively, other Cysteine residues can be added to the peptide amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS). PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1:acryloyl chloride 1.5:triethylamine 2, at 0.2 gram PEG/mL DCM). Such chemical groups can be attached to linearized, 2-arm, 4-arm, or 8-arm PEG molecules.

Resultant conjugated molecules (e.g., PEGylated or PVP-conjugated polypeptide) are separated, purified and qualified using e.g., high-performance liquid chromatography (HPLC) as well as biological assays.

According to another embodiment, the peptide or proteinaceous agent is attached to a sustained-release enhancing agent. Exemplary sustained-release enhancing agents include, but are not limited to, hyaluronic acid (HA), alginic acid (AA), polyhydroxyethyl methacrylate (Poly-HEMA), polyethylene glycol (PEG), glyme and polyisopropylacrylamide.

According to specific embodiments, the peptide is presented in context of an antigen presenting cell. The most common cells used to load antigens are bone marrow and peripheral blood derived dendritic cells (DC), as these cells express co-stimulatory molecules that help activation of CTL. Nevertheless, the peptide presenting cell can also be a macrophage, a B cell or a fibroblast. According to specific embodiments, the antigen presenting cell is a dendritic cell. Presenting the peptide can be effected by a variety of methods, such as, but not limited to, transforming the presenting cell with the polynucleotide encoding the peptide; loading the presenting cell with the peptide. Loading can be external or internal.

The present invention further encompasses using the peptides in obtaining the agents disclosed herein.

Thus, according to an aspect of the present invention there is provided a method of obtaining an agent of interest, the method comprising using the modified or unmodified peptide disclosed herein for producing or selecting an agent specifically recognizing said peptide, thereby producing the agent of interest.

Thus as non-limiting examples, the method comprising immunization using the modified or unmodified peptide disclosed herein for producing an antibody of interest, or phage display for antibody selection.

The therapeutics agents (e.g. peptides, agents or cells) of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide, agent or cell accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

According to specific embodiments, the pharmaceutical composition comprises an adjuvant.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution. Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agent, cell) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

In addition, existing or induced immune response to the agents and/or cells disclosed herein can be tested using e.g. multimers assays, intracellular cytokines release or CTL assays.

Dosage amount and interval may be adjusted individually to provide that the levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

It will be appreciated that the therapeutic agents of the present invention can be provided to the individual in combination with each other and/or with additional active agents to achieve an improved therapeutic effect as compared to treatment with each agent by itself. Thus, for example, combination of different agents that match the different HLA alleles of the patients can be used.

In such therapy, measures (e.g., dosing and selection of the complementary agent) are taken to adverse side effects which may be associated with combination therapies.

Administration of such combination therapy can be simultaneous, such as in a single capsule having a fixed ratio of these active agents, or in multiple capsules for each agent.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiments, the therapeutic agent disclosed herein (e.g. the peptide, agent and/or cell expressing same) can be administered to a subject with other established or experimental therapeutic regimen to treat cancer including analgetics, chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy, photon beam radiosurgical therapy and other treatment regimens which are well known in the art.

According to an aspect of the present invention there is provided an article of manufacture comprising the peptide, the agent or the cell disclosed herein and a cancer therapy.

According to specific embodiment, the, peptide, the agent or the cell disclosed herein and the cancer therapy are packaged in separate containers.

According to specific embodiment, the peptide, the agent or the cell disclosed herein and the cancer therapy are packaged in a co-formulation.

According to specific embodiments, the article of manufacture is identified for the treatment of cancer.

As the identified MHC presented modified and un-modified peptides have been identified by the present inventors as cancer antigens, specific embodiments of the present invention further propose analyzing for the presence and/or level of such presented peptides for the purpose of diagnosing and/or monitoring treatment efficacy.

Hence, according to an aspect of the present invention, there is provided a method of detecting a cancer cell in a subject, the method comprising determining in a biological sample of the subject a cell surface a level of a peptide having an amino acid sequence selected from the group consisting of SEQ ID NO: 1-10746, 10817, 10819, 10820, 10823, 10824, 10826 and 10827 and the corresponding modification according to Table 3 hereinabove, wherein a level of said peptide above a predetermined threshold and/or increased level relative to a reference biological sample of a healthy subject is indicative of presence of cancer cell in said subject, thereby detecting cancer cell in the subject.

According to an additional or an alternative aspect of the present invention, there is provided a method of detecting a cancer cell in a subject, the method comprising determining in a biological sample of the subject a cell surface a level of a peptide selected from the group consisting of SEQ ID NO: 10747-10816 and 10822, wherein a level of said peptide above a predetermined threshold and/or increased level relative to a reference biological sample of a healthy subject is indicative of presence of cancer cell in said subject, thereby detecting cancer cell in the subject.

According to specific embodiments, the presence of the peptide on the cell surface of a cell is indicative of the cancer.

According to specific embodiments, the level of the peptide on the cell surface of a cell is indicative of the cancer.

According to specific embodiments, a level above a predetermined threshold is indicative of cancer.

According to an additional or an alternative aspect of the present invention, there is provided a method of treating cancer in a subject in need thereof, the method comprising detecting the cancer according to the method, and wherein presence of cancer is indicated, treating the subject with a cancer therapy.

According to specific embodiments, the cancer therapy comprises the peptide, the agent or cells disclosed herein.

According to an additional or an alternative aspect of the present invention, there is provided a method of monitoring efficacy of cancer therapy in a subject, the method comprising determining in a biological sample of the subject a cell surface level of a peptide having an amino acid sequence selected from the group consisting of SEQ ID NO: 1-10746, 10817, 10819, 10820, 10823, 10824, 10826 and 10827 and the corresponding modification according to Table 3 hereinabove following the cancer therapy, wherein a decrease from a predetermined threshold in the level of said peptide following the cancer therapy indicates efficaciousness of the cancer therapy.

According to an additional or an alternative aspect of the present invention, there is provided a method of monitoring efficacy of cancer therapy in a subject, the method comprising determining in a biological sample of the subject a cell surface level of a peptide selected from the group consisting of SEQ ID NO: 10747-10616 and 10822 following the cancer therapy, wherein a decrease from a predetermined threshold in the level of said peptide following the cancer therapy indicates efficaciousness of the cancer therapy.

On the other hand, if there is no change in the cell surface level of the peptide, or in case there is an increase in the level of cell surface amount of the peptide, then the cancer therapy is not efficient in treating the cancer and additional and/or alternative therapies (e.g., treatment regimens) may be used.

According to specific embodiments of the monitoring aspects disclosed herein, the predetermined threshold is in comparison to the level in the subject prior to cancer therapy.

According to specific embodiments, the decrease from a predetermined threshold is statistically significant.

According to specific embodiments of the monitoring aspects disclosed herein, the decrease from a predetermined threshold is at least 1.5 fold, at least 2 fold, at least 3 fold, at least fold, at least 10 fold, or at least 20 fold as compared the level in a control sample prior to the cancer therapy as measured using the same assay.

According to specific embodiments, the decrease from a predetermined threshold is at least 2%, at least 5%, at least 10%, at least 20%, at least 30%, at least 40%, at least 50%, at least 60%, at least 70%, at least 80%, at least 90%, e.g., 100%, at least 200%, at least 300%, at least 400%, at least 500%, at least 60) % the level in a control sample prior to the cancer therapy as measured using the same assay.

According to other specific embodiments of the monitoring aspect of the present invention, the pre-determined threshold can be determined in a subset of subjects with known outcome of cancer therapy.

According to specific embodiments, determining cell surface amount of the peptide is effected in-vitro or ex-vivo.

Non-limiting examples of biological samples include, but are not limited to, a cell obtained from any tissue biopsy, a tissue, an organ, body fluids such as blood, and rinse fluids.

The biological sample can be obtained using methods known in the art such as using a syringe with a needle, a scalpel, fine needle biopsy, needle biopsy, core needle biopsy, fine needle aspiration (FNA), surgical biopsy, buccal smear, lavage and the like. According to specific embodiments, the biological sample is obtained by biopsy.

Methods of determining cell surface amount are known in the art, and include e.g. flow cytometry, immunohistochemistry and the like, which may be effected using e.g. antibodies specific to MHC presented peptide.

According to specific embodiments, the determining is performed by contacting the biological sample with an agent capable of detecting the MHC presented peptide, e.g. an antibody.

According to specific embodiments, the contacting is effected under conditions which allow the formation of a complex comprising MHC presented peptide present in the biological sample and the agent (e.g. immunocomplex).

The complex can be formed at a variety of temperatures, salt concentration and pH values which may vary depending on the method and the biological sample used and those of skills in the art are capable of adjusting the conditions suitable for the formation of each complex.

Thus, according to an additional or an alternative aspect of the present invention, there is provided a composition of matter comprising a biological sample of a subject, and an agent capable of detecting a MHC presented peptide having an amino acid sequence selected from the group consisting of SEQ ID NO: 1-10746, 10817, 10819, 10820, 10823, 10824, 10826 and 10827 and the corresponding modification according to Table 3 hereinabove.

According to an additional or an alternative aspect of the present invention, there is provided a composition of matter comprising a biological sample of a subject, and an agent capable of detecting a MHC presented peptide selected from the group consisting of SEQ ID NO: 10747-10816 and 10822.

According to an aspect of the present invention there is provided an article of manufacture comprising a biological sample of a subject, and in a separate container an agent capable of detecting a MHC presented peptide having an amino acid sequence selected from the group consisting of SEQ ID NO: 1-10746, 10817, 10819, 10820, 10823, 10824, 10826 and 10827 and the corresponding modification according to Table 3 hereinabove.

According to an aspect of the present invention there is provided an article of manufacture comprising a biological sample of a subject, and in a separate container an agent capable of detecting a MHC presented peptide selected from the group consisting of SEQ ID NO: 10747-10816 and 10822.

According to specific embodiments, the methods disclosed herein comprise corroborating the diagnosis using a state of the art technique.

Such methods are known in the art and depend on the cancer type and include, but not limited to, complete blood count (CBC), tumor marked tests (also known as biomarkers), imaging (such as MRI. CT scan. PET-CT, ultrasound, mammography and bone scan), endoscopy, colonoscopy, biopsy and bone marrow aspiration.

An additional or an alternative aspect of some embodiments relates to systems, methods, an apparatus, and/or code instructions (e.g., stored on a memory and executable by one or more hardware processors) for generating a dataset of post translations modifications (PTM) on major histocompatibility complex (MHC) bound peptides. The systems, methods, apparatus, code instructions may generate the dataset of PTMs on MHC bound peptides described herein. A mass spectrometry (MS) dataset is obtained from a sample of cells associated with a target disease for treatment, where exemplary diseases are for example, as described herein. The dataset stores spectra data elements outputted by a MS device analyzing MHC bound peptides to generate amino acid sequences. Each spectra data element for a respective amino acid sequence of the MHC bound peptides. A reference sequence dataset storing amino acid sequences of proteins is received. A variable modification dataset storing modifications each including a respective amino acid and expected mast shift is received. Multiple combinations are generated, where each combination includes a respective amino acid sequence selected from the reference sequence dataset and at least one modification selected from the variable modification dataset. A parallel search task is executed on multiple processors connected in parallel and/or in a distributed processing computational architecture. Each processor searches for a respective spectra element of the combinations to identify multiple best peptide to spectra matches (PSMs). Each respective processor assigns a ranking score to each respective PSM according to the respective search performed by the respective processor. The PSMs from the multiple processors connected in parallel are aggregated to generate a main PSM list. The main PSM list includes main ranking scores, which are computed from the ranking score of each respective PSM of each respective search. Highest ranking PSMs are selected according to respective main ranking scores. In a modified sequence dataset, modified sequences each including the PTM and sequences corresponding to the selected highest ranking PSMs are stored. The modified sequence dataset stores an indication of binding motifs defined by multiple identified PTMs and corresponding sequence. The modified sequence dataset is provided for selecting a certain binding motif having a certain PTM and corresponding amino acid sequence from the modified sequence dataset capable of specifically binding an MHC presented peptide for treatment of the target disease.

Optionally, this highest ranking PSMs are further prioritized for inclusion in the modified sequence dataset. Multiple quality assignment measures may be computed, and one or more of the following may be performed using the quality assignment measures: validating the PTM of each member of the PSM aggregation dataset according to the quality measures, filtering ambiguous assignments and isobaric decoys of the PSM aggregation dataset according to a filtering threshold, ranking members of the PSM aggregation dataset, and selecting the highest ranking PSMs according to the highest ranked member of the PSM aggregation dataset.

Optionally, a training dataset is created by labelling each modified sequence of the modified sequence dataset with an indication of one or more of: an MHC type, parent gene, and position of the motif within a full protein length, and includes an amino acid sequence. PTM type, and position of the PTM on the amino acid sequence. A machine learning (ML) model is trained using the training dataset. For an input of a certain modified sequence defined by a combination of an amino acid sequence and at least one PTM into the ML model, an indication of whether the certain modified sequence is predicted to fit a binding motif that binds to a cell of the MHC type is obtained as an outcome of the ML model. Alternatively or additionally, for an input of an amino acid sequence of a full protein length and PTMs into the ML model, at least one modified sequence predicted to fit a binding motif is obtained as an outcome of the ML model.

Treatments for the target disease may be created using the modified sequence dataset, as described herein.

Exemplary machine learning models, as described herein, may include one or more classifiers, neural networks of various architectures (e.g., fully connected, deep, encoder-decoder), support vector machines (SVM), logistic regression, k-nearest neighbor, decision trees, boosting, random forest, and the like. Machine learning models may be trained using supervised approaches and/or unsupervised approaches.

At least some implementations of the systems, methods, apparatus, and/or code instructions described herein address the technical problem of identifying PTMs in endogenous peptides, optionally, improving spectral assignment rates in mass spectrometry (MS) data of endogenous peptides. At least some implementations of the systems, methods, apparatus, and/or code instructions described herein address the technical problem of identifying motifs that are predicted to bind to MHC of cells. At least some implementations of the systems, methods, apparatus, and/or code instructions described herein improve the technical and/or medical field of immunotherapy, by providing computer implemented methods for predicting motifs that bind to MHC of diseased cells (e.g., cancer) which may be used to create immunotherapy for treating the disease.

At least some implementations of the systems, methods, apparatus, and/or code instructions described herein improve the technical and/or medical field of machine learning, by creating ML models that predict motifs that bind to certain cells, which may be used to create immunotherapy for treating a disease of the cells. For example, in an analysis of patient cohorts (e.g. as described with reference to Bassani-Sternberg. M. et al. Direct identification of clinically relevant neoepitopes presented on native human melanoma tissue by mass spectrometry. Nat. Commun. 7, (2016), Chong, C. et al. High-throughput and Sensitive Immunopeptidomics Platform Reveals Profound Interferon γ-Mediated Remodeling of the Human Leukocyte Antigen (HLA) Ligandome. Mol. Cell. Proteomics 17, 533-548 (2018), and/or Ternette. N. et al. Immunopeptidomic Profiling of HLA-A2-Positive Triple Negative Breast Cancer Identifies Potential Immunotherapy Target Antigens. Proteomics 18, 1700465 (2018), cell lines (e.g., as described with reference to Bassani-Sternberg. M., Pletscher-Frankild, S., Jensen, L. J. & Mann, M. Mass Spectrometry of Human Leukocyte Antigen Class I Peptidomes Reveals Strong Effects of Protein Abundance and Turnover on Antigen Presentation. Mol. Cell. Proteomics 14, 658-673 (2015) and/or Shraibman, B., Kadosh, D. M., Barnea, E. & Admon, A. Human Leukocyte Antigen (HLA) Peptides Derived from Tumor Antigens Induced by Inhibition of DNA Methylation for Development of Drug-facilitated Immunotherapy. Mol. Cell. Proteomics 15, 3058-3070 (2016)), and mono-allelic (e.g., as described with reference to Abelin, J. G. et al. Mass Spectrometry Profiling of HLA-Associated Peptidomes in Mono-allelic Cells Enables More Accurate Epitope Prediction. Immunity 46, 315-326 (2017)) performed by Inventors using embodiments described herein, HLA immunopeptidomics data reveal that modifications generate novel HLA I binding motifs that could not be identified merely by the amino acid sequence. This finding suggests that existing HLA I binding predictors tools (e.g., as described with reference to Abelin, J. G. et al. Mass Spectrometry Profiling of HLA-Associated Peptidomes in Mono-allelic Cells Enables More Accurate Epitope Prediction. Immunity 46, 315-326 (2017), Jurtz, V. et al. NetMHCpan-4.0: Improved Peptide-MHC Class I Interaction Predictions Integrating Eluted Ligand and Peptide Binding Affinity Data. J. Immunol. 199, 3360-3368 (2017), Gfeller, D. et al. The Length Distribution and Multiple Specificity of Naturally Presented HLA-I Ligands. J. Immunol. 201, 3705-3716 (2018), Bulik-Sullivan, B. et al. Deep learning using tumor HLA peptide mass spectrometry datasets improves neoantigen identification. Nat. Biotechnol. 37, 55-71 (2019), and/or O'Donnell, T. J., Rubinsteyn, A. & Laserson, U. MHCflurry 2.0: Improved Pan-Allele Prediction of MHC Class 1-Presented Peptides by Incorporating Antigen Processing. Cell Syst. 11, 42-48.e7 (2020)) are “blind” to those motifs and purely predict epitopes that contain highly modified amino-acid like cysteine (e.g., as described with reference to Rev, A. et al. Immunoinformatics: Predicting Peptide—MHC Binding). An improved HLA I predictor ML tool is established by training a machine learning module based on a training dataset created from the dataset generated by at least some embodiments described herein that include, for example, unique modified HLA I bound peptides dataset. The training dataset may include, for example, peptide-intrinsic features such as the peptide sequence, the modification type, and position. The training dataset may further incorporate extrinsic features such as the HLA type, parent gene, and known modification sites. The ML model classifies the input modified peptide as a predicted binder/nonbinder to specific HLA haplotype, and/or may suggest the modified potential binders out of a full protein length and a list of modification types.

The technical problem of identifying PTMs in endogenous peptides arises since almost all proteins are known to be modified in a specific biological context [27] but in a global PTM discovery analysis, only parts of them will be modified. The relative abundance of PTM is lower as the PTMs are sub-stoichiometric, making the PTMs difficult to detect. One existing approach to overcome the under-representation of modified peptides prior to MS analysis is using biochemical methods to enrich the sample for a specific PTM of interest. However, the disadvantage of this approach is that the enrichment step requires more material to start with (challenging in a clinical setting) and typically enriches only specific modifications, making it less suitable for diverse, global PTM analysis. At least some implementations of the systems, methods, apparatus, and/or code instructions described herein are sensitive enough to allow for rapid and combinatorial detection of multiple PTMs without prior biochemical enrichment. Enrichment steps will identify more modification site for a specific type of PTM while a broad analysis will capture better the biological stoichiometry and potential cross-talk between modification types.

There are major conceptual differences when searching for endogenous peptides (e.g., HLA I peptide) versus performing proteolytic peptide analysis using mass spectrometry (e.g., using the commonly used trypsin, for example, as described with reference to Park, C. Y., Klammer, A. A., Käli, L, MacCoss, M. J. & Noble, W. S. Rapid and accurate peptide identification from tandem mass spectra. J. Proteome Res. 7, 3022-3027 (2008)). In the latter, an expected pattern for cleaved peptides is predicted based on the ability of trypsin to cleave c-terminal to lysine or arginine residues, thereby generating specific termini. Usually, one can settle for two or more unique peptides to infer the existence of a protein in the sample and more than three hits will give a good estimation of the relative abundance of the unique peptide. Most of the time, a protein will have multiple peptides from different regions, which makes the identification more robust against false discoveries. The technical challenge, which is addressed and solved by at least some implementations of the systems, methods, apparatus, and/or code instructions described herein, arises when searching for an endogenous peptide with no known cleavage sites, where the peptide itself the search target. That is why the approach requires a specific search for each potential peptide with an unspecified cleavage.

The challenges of identifying PTMs on mass spectrometry data and its effect on the search space is described, for example, in a review described with reference to Na, S. & Paek, E. Software eyes for protein post-translational modifications. Mass Spectrom. Rev. 34, 133-147 (2015). When combining multiple potential PTMs and endogenous peptides, exponential growth of the search space results, making search times impractical. The enormous search space causes an over-fitting of matched peptides and makes it difficult to distinguish between true and false peptides identification (e.g., as described with reference to Verheggen, K. et al. Anatomy and evolution of database search engines—a central component of mass spectrometry based proteomic workflows. Mass Spectrom. Rev. 1-15 (2017), doi:10.1002/mas.21543). As such, applying a false discovery rate (FDR) of 1%, as often used for bottom-up proteomics, will decrease the total number of peptide identification. Existing tools use de novo mass spectrum interpretations to create short peptide tags and then combine those tags to a full-length sequence by searching against a reference proteomics dataset, prioritizing unmodified solution and relaying on tryptic peptide characteristics (for example, PEAKs, TagGraph (e.g., as described with reference to Devabhaktuni, A. et al. TagGraph reveals vast protein modification landscapes from large tandem mass spectrometry datasets. Nat. Biotechnol. 37. (2019)). Other tools use external datasets of known modification to run a sequential assignment strategy starting with unmodified sequences and follow-up by known modification sites and then match novel modification (e.g., MetaMorpheus as described with reference to Solntsev, S. K., Shortreed, M. R., Frey. B. L. & Smith, L. M. Enhanced Global Post-translational Modification Discovery with MetaMorpheus. J. Proteome Res. 17, 1844-1851 (2018)). Using existing approaches, existing sequence database searching algorithms create all the possible peptide candidates from a given reference sequence (in-silico digestion), convert them to a theoretical spectrum, compare them to the experimental spectra and calculate a matching score. Adding potential modifications and non-canonical sequences to the theoretical search space exponentially increase the number of peptide possibilities, making search times a limiting factor. At least some implementations of the systems, methods, apparatus, and/or code instructions described herein address the technical problem of increased search time, and provide a solution that provide a reasonable search time, even for extremely large number of possible combinations that are being searched, by using a parallel processing architecture while allowing each spectra assignment (also referred to herein as MS data element) to be tested against any other. At least some implementations of the systems, methods, apparatus, and/or code instructions described herein address the technical problem of false identification, by a prioritization phase that uses quality assignment measures that reduce false identification. At least some implementations of the systems, methods, apparatus, and/or code instructions described herein include proteoforms with PTM in the peptide search space.

At least some implementations of the systems, methods, apparatus, and/or code instructions described herein provide improvements over existing approaches. For example, in one approach, multiple PTM searches are performed using a sequential assignment. The first assignment is for unmodified peptides. Only spectra that were not assigned in the first phase are considered for modification assignment. Another approach based on sequential assignment uses an external database of known modification sites to search for those in the first phase. Such approaches miss some PTMs. At least some implementations of the systems, methods, apparatus, and/or code instructions described herein are able to find the PTMs missed by this approach. In particular, sequential assignment is not applied. Inventors compared the identifications using embodiments described herein, to those from a standard search (only n-acetylation and methionine oxidation included). Out of the peptide to spectrum matches (PSMs) which conflicted between the two searches (1.22% of PSMs), 67% received a higher scoring match in the multi-modification search. This is a feature of at least some embodiments described herein that allows for better scoring matches to replace previous assignments which cannot happen in sequential search software. On average, the match score was increased by 13%, although score alone is not a guarantee of a true assignment it does suggest the inclusion of a modification in the predicted peptide better described the spectrum.

Another approach is based only on tryptic digested protein samples, and not HLA peptides. Using trypsin to digest the sample before mass spectrometry analysis allows any matching algorithm to narrow its search space to peptides that are cleaved after lysine or arginine and not before proline. However, when trying to identify endogenous peptides that were not solely cleaved by trypsin, such as in the case of HLA, the cleavage terminus is not restricted and the number of theoretical peptides increases dramatically. Such approaches cannot process peptides cleaved using other approaches.

At least some embodiments described herein enable finding PTM using proteins cleaved with any and/or unknown approaches, using the distributed and/or parallel computational architecture, which is scalable, and provides no known boundaries to the size of the reference data and/or number of PTMs. A conceptually “unlimited” number of PTMs and/or reference dataset sizes enables explore any combination and/or cross-talk between PTMs. The MHC and/or HLA bounded peptides contain a large variety of PMS and some peptides have more than one PMS. At least some embodiments described herein perform a systematic search that identify more of those peptides and their PTMs.

At least some implementations of the systems, methods, apparatus, and/or code instructions described herein address the technical problems described herein, improve the technical field as described herein, and/or improve over existing approaches described herein, for example, using one or more of the following features of at least some embodiments described herein:
Using a two stages, a matching phase, and a prioritizing phase—The matching phase reduces the running time by distributing the matching feature across parallel processing clusters. The merge process of each distributed task allows ranking the peptide to spectra (PSM) assignment from each instance like they were executing on a single search. The prioritizing phase includes several computational steps to validate the PTM identification, filter ambiguous assignment, and isobaric decoys, and help rank the prediction by their quality.
Merge feature—when running multiple instances of a matching process that matches the MS data elements to a reference dataset of combinations of protein sequences and PTM, each instance provides its respective best match. But each instance searches a different subset of the reference data set and for a different combination of PTMs. As a result, each instance generates a different assignment list with a different expectation score, for example, based on the score histogram calculated for the respective search results. The merge feature described herein compares the results from the different instances and reconstructs the score histogram to recalculate the expectation score.
Lower rank identification feature—the increased search space creates overfitting of the data and makes it harder to distinguish between true and false identification. In embodiments described herein, this is shown by getting several good assignments with a very similar score. Other approaches take the best score even if the delta score to the next fit (lower ranks) is negligible. In at least some embodiments, all the matches that are in a 5% (or other defined value, for example, 1%, 3%, 7%, 10%, or other) delta score from the leading hit are identified, and used for computing the quality measurements in the prioritizing features. This feature lowers the negative effect of overfitting of the data.
Modification decoys based on PTM localization window and mass shift—Addresses the technical problem of automating how an expert manually assesses the spectra assignment to a peptide. The manual process is not simply automated, but includes new features that are not and cannot be performed manually, and are not part of any existing automated process. An expert evaluation is one of the most trusted methods to evaluate a spectra assignment and broadly used in research. While an expert invests an average of 30 min per spectra, which is impractical for generating an automated process, at least some embodiments described herein performs them automatically, by includes the one or more of the following features in the prioritizing phase: spectrum annotation, PTM localization, search for mass decoys and/or isobaric masses and search mass boundary effect bias. The annotation feature may implement third-party tools but increases its capabilities dramatically. The annotation is used for PTM validation.
Search for mass decoys or isobaric masses—all alternative theoretical solution for a specific PTM site are considered, even a solution that was not in the original search criteria. Search mass boundary effect bias—a unique problem when searching for PTMs.
Combined weighted scoring—the measurements collected per spectrum in the priority phase may be aggregated and/or considered, to determine whether a certain match is valid a potential decoy.
Enrichment feature—the information gathered during the prioritizing phase enables performing unique enrichment steps when comparing samples.
Predictor on a unique dataset—the quality dataset of modified immunopeptidomics including previously undiscovered PTMs enables creating a new ML predictor process.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk. C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

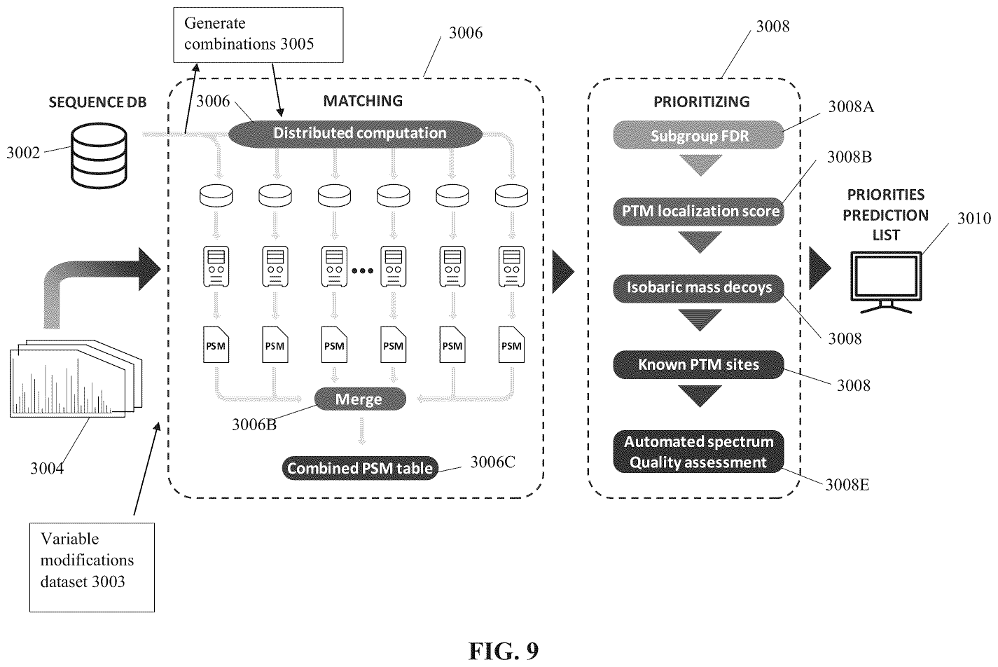

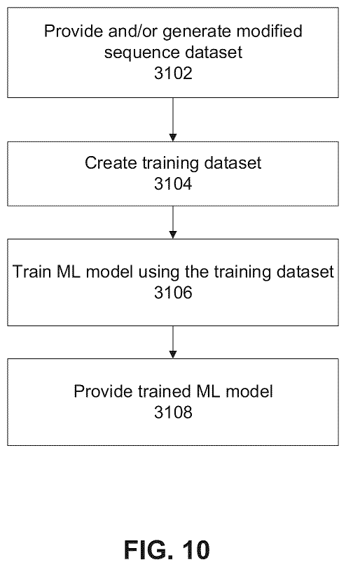

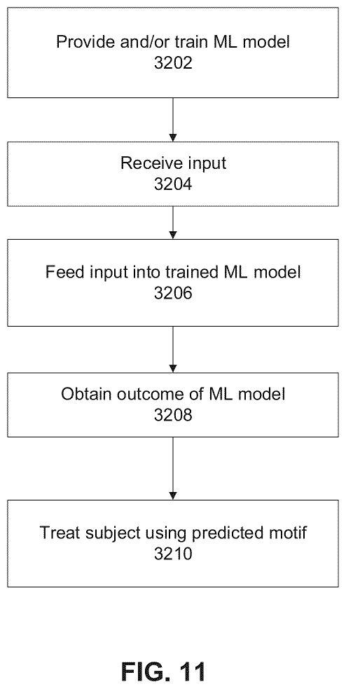

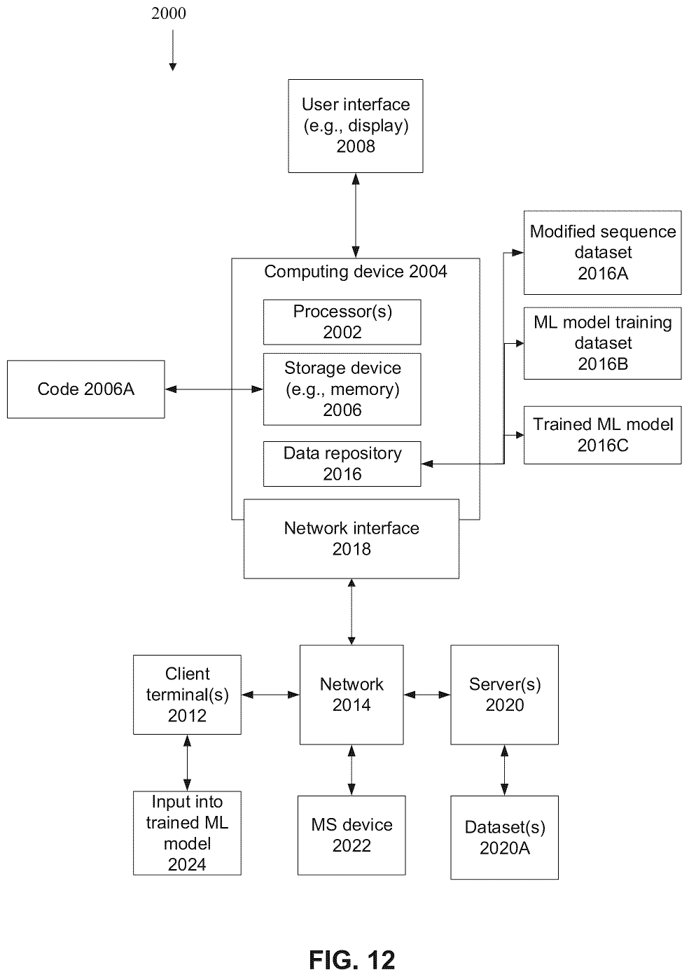

Reference is now made to FIG. 9, which is a flowchart of an exemplary process for generating a modified sequence dataset storing an indication of binding motifs defined by multiple PTM and corresponding sequence, in accordance with some embodiments of the present invention. A certain binding motif having a certain PTM and corresponding amino acid sequence selected from the modified sequence dataset is predicted to be capable of specifically binding an MHC presented peptide for treatment of a target disease. Reference is also made to FIG. 10, which is a flowchart of an exemplary process for generating an ML model using the modified sequence dataset, in accordance with some embodiments of the present invention. Reference is also made to FIG. 11, which is a flowchart of an exemplary process for using the ML model trained using the modified sequence dataset, in accordance with some embodiments of the present invention. Reference is also made to FIG. 12, which is a block diagram of a system 2000 for generating the modified sequence dataset and/or training the ML model on the modified sequence dataset and/or using the ML model trained on the modified sequence dataset, in accordance with some embodiments of the present invention.

System 2000 may implement the acts of the method described with reference to FIGS. 9, 10, and/or 11, by processor(s) 2002 of a computing device 2004 executing code instructions 2006A stored in a storage device 2006 (also referred to as a memory and/or program store).

Computing device 2004 may be implemented as, for example, a client terminal, a server, a computing cloud, a virtual server, a virtual machine, a mobile device, a desktop computer, a thin client, a Smartphone, a Tablet computer, a laptop computer, a wearable computer, glasses computer, and a watch computer.

Multiple architectures of system 2000 based on computing device 2004 may be implemented. In an exemplary implementation, computing device 2004 storing code 2006A, may be implemented as one or more servers (e.g., network server, web server, a computing cloud, a virtual server) that provides services (e.g., one or more of the acts described with reference to FIG. 9. FIG. 10, and/or FIG. 11) to one or more client terminals 2012 over a network 2014, for example, providing software as a service (SaaS) to the client terminal(s) 2012, providing software services accessible using a software interface (e.g., application programming interface (API), software development kit (SDK)), providing an application for local download to the client terminal(s) 2012, and/or providing functions using a remote access session to the client terminals 2012, such as through a web browser. For example, computing device 2004 generates a modified sequence dataset 2106A, which is used to generate an ML model training dataset 2106B for generating a trained ML model 2106C, as described herein. Multiple users use their respective client terminals 2012 to access computing device 2004, which may be remotely located. Client terminal 2012 provides input data for feeding into the trained ML model 2024 to computing device 2004, for example, via the API, and/or via an application locally installed on client terminal 2012, and/or by another file transfer protocol. Computing device 2004 centrally inputs data 2024 into trained ML model 2016C to generate an outcome, as described herein. Computing device 2004 may provide the outcome of trained ML model 2106C to respective client terminal 2012 (corresponding to each data 2024) for presentation on a display associated with client terminal 2012. In another example, computing device 2004 may include locally stored software (e.g., code 2006A) that performs one or more of the acts described with reference to FIG. 9, FIG. 10, and/or FIG. 11, for example, as a self-contained system such as a laboratory server in communication with MS device 2022. Code 2006A may be implemented as a plug-in and/or additional feature set for integration with existing software that controls MS device 2022.

Processor(s) 2002 of computing device 2004 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 2002 may include multiple processors (homogenous or heterogeneous) arranged for parallel processing, as clusters and/or as one or more multi core processing devices. Processor(s) 2002 may be arranged as a distributed processing architecture, for example, in a computing cloud, and/or using multiple computing devices. Processor(s) 2002 may include a single processor, where optionally, the single processor may be virtualized into multiple virtual processors for parallel processing, as described herein.

Data storage device 2006 stores code instructions executable by processor(s) 2002, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Storage device 2006 stores code 2006A that implements one or more features and/or acts of the method described with reference to FIG. 9, FIG. 10, and/or FIG. 11 when executed by processor(s) 2002.

Computing device 2004 may include a data repository 2016 for storing data, for example, storing one or more of a modified sequence dataset 2016A generated as described with reference to FIG. 9 and/or including data as described herein, ML model training dataset 2016B created from modified sequence dataset 2016A as described herein, and/or trained ML model 2016C created as described with reference to FIG. 10 and/or used as described with reference to FIG. 11. Data repository 2016 may be implemented as, for example, a memory, a local hard-drive, virtual storage, a removable storage unit, an optical disk, a storage device, and/or as a remote server and/or computing cloud (e.g., accessed using a network connection).

Computing device 2004 may include a network interface 2018 for connecting to network 2014, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 2014 may be implemented as, for example, the internet, a local area network, a virtual private network, a wireless network, a cellular network, a local bus, a point to point link (e.g., wired), and/or combinations of the aforementioned.

Computing device 2004 may connect using network 2014 (or another communication channel, such as through a direct link (e.g., cable, wireless) and/or indirect link (e.g., via an intermediary computing unit such as a server, and/or via a storage device) with one or more of:
Server(s) 2020 storing one or more dataset(s) 2020A, for example, a MS dataset obtained from a sample of cells associated with a target disease for treatment, a reference sequence dataset storing amino acid sequences of proteins, a variable modification dataset storing modifications each including a respective amino acid and expected mast shift, and a dataset of known PSM of healthy cells and cells with the target disease, as described herein.
Mass spectrometry (MS) device 2022 that generates spectra data elements, as described herein.
Client terminals 2012, which may provide data for input 2024 into trained ML model 2016C, as described herein.

Computing device 2004 and/or client terminal(s) 2012 include and/or are in communication with one or more physical user interfaces 2008 that include a mechanism for a user to enter data (e.g., provide the data 2024 for input into trained ML model 2016C) and/or view the displayed outcome of ML model 2016C, optionally within a GUI. Exemplary user interfaces 2008 include, for example, one or more of, a touchscreen, a display, a keyboard, a mouse, and voice activated software using speakers and microphone.

Referring now back to FIG. 9, at 3002, a reference sequence dataset storing amino acid sequences of proteins is received. The proteome reference sequence file may be represented, for example, in the fasta format.

At 3003, a variable modification dataset storing multiple modifications each including a respective amino acid and expected mast shift is received.

At 3004, a mass spectrometry (MS) dataset obtained from a sample of cells associated with a target disease for treatment is received. Target diseases may be, for example cancer, autoimmune related diseases (e.g., Crohn's, arthritis), and others, as described herein. The MS dataset includes spectra data elements outputted by a MS device analyzing MHC bound peptides to generate amino acid sequences. The peptides may be generated by cleaving proteins using one or more enzymes, which may not be known, for example, including and/or excluding trypsin. Each spectra data element is for a respective amino acid sequence of the MHC bound peptides. The spectra data elements may be represented, for example, as MS raw files such as in the mzML format.

At 3005, multiple combinations are generated. Each combination includes a respective amino acid sequence selected from the reference sequence dataset and at least one modification selected from the variable modification dataset.

At 3006, a search is performed in parallel, using multiple parallel processors, for example, as described with reference to 3006A-C. The search may be divided so that each processor searches through a different search space. The spectra data elements may be divided so that each processor searches a different subset of the spectra data elements. Each processor may search its subset of the spectra data elements on the entire set of generated combination, and/or on a subset of the generated combinations.

Optionally, each processor searches for a respective spectra element of the multiple combinations to identify a set of best peptide to spectra matches (PSMs). Each respective processor assigns a ranking score to the respective PSM according to the respective search performed by the respective processor. It is noted that the technical problem described herein of creating a main PSM list arises since each processor assigns its own ranking score based on its own search, which is performed using different data. The spectra element(s) searched by each processor, may be conceptually through of a puzzle of MHC bound proteins that are cleaved to generate puzzle pieces of the peptides. Each processor searches the puzzle pieces, which makes it technically challenging to arrange the puzzle pieces together without knowing what the puzzle (i.e., protein) is. In other words, the parallel processing is not simply taking a search query and dividing the search task into parallel processing, but taking the search query, splitting it up into different components, and then searching the components without necessarily knowing what the original search query is.

At 3006A, a respective subset of the combinations (or all combinations) may be allocated to processors connected for parallel processing, where each respective processor searches its respective allocated spectra elements on the respective subset of (or all) combinations to identify a respective set of PSM.

A single search task may be distributed into thousands of instances that are performed in parallel on a CPU cluster, for example, a search process that creates all the possible peptide candidates from a given reference sequence (in-silico digestion), converts them to a theoretical spectrum, compares them to the experimental spectra and calculates a matching score, for example, MSFragger, for example, as described with reference to Andy T. Kong1, 2, Felipe V. Leprevost2. Dmitry M. Avtononmov2, D. M. & Nesvizhskii, and A. I. MSFragger: ultrafast and comprehensive peptide identification in shotgun proteomics. 14, 39-46 (2017). The search tasks may be split by dividing the search into batches and the list of variable modifications into each potential combination up to, for example, 5, 6, 7, 8, or other number of mass shifts per instance.

At 3006B, the respective set of PSM of each respective processor is merged to create a PSM aggregation dataset.

As discussed herein, merging the PSM datasets is a technical challenge, where for example, statistical parameters used in a subsequent false discovery rate (FDR) calculation feature (e.g., as described with reference to 3008A) are distorted by multiple searches of a same reference dataset over different software instances executed by the multiple parallel connected processors. To address this technical challenge, in at least some implementations, the merge process uses unmodified hits combined histogram to evaluate the number of duplicated hits and remove the duplicates. The merge process may recalculate the expectation based on the restored score histogram for each PSM. The merge process aggregates the individual search results to help assure accurate FDR calculation in the prioritizing stage (e.g., feature 3008).

The merging may be performed by removing duplicated PSM from the PSM aggregation dataset, for example, by using unmodified hits combined histogram to evaluate a number of duplicated PSM and identify the duplicated PSM for removal thereof. An expectation based on a restored score histogram for each PSM is recalculated. The merge process assembles the different output results obtained from each process executing on each parallel connected processor, prioritizing the best peptide to spectra match (PSM) solution, for example, according to its hyperscore and/or minimum delta masses.

At 3006C, the PSMs results from the processors connected in parallel are aggregated to generate a main PSM list with main ranking score. The main PSM list may be generated by computing the main ranking score from the ranking score of each respective PSM of each respective search performed by each respective parallel connected processor. Highest ranking PSMs are selected according to respective main ranking scores.

The highest ranking PSMs may be selected from the PSM aggregation dataset, for example, PSMs above a selected threshold and/or a top number of PSMs (e.g., top 100, or 500, or 1000 or other number), and/or top percentage of PSMs (e.g., top 1%, or 5%, or 10%, or other percentage).

At 3008, an optional prioritization process, including one or more optional features, is executed. The highest ranking PSMs may be further prioritized for inclusion in the modified sequence dataset.

The prioritization process collects a set of quality assignment measurements and uses the set of quality assignment measures to filter ambiguous assignments and potentially false identifications, for example, as described with reference to 3008A-E. It is noted that one or more of 3008A-E may be included and/or excluded from the process.

Multiple quality assignment measures may be computed, and one or more of the following may be performed using the quality assignment measures: validating the PTM of each member of the PSM aggregation dataset according to the quality measures, filtering ambiguous assignments and isobaric decoys of the PSM aggregation dataset according to a filtering threshold, ranking members of the PSM aggregation dataset, and selecting the highest ranking PSMs according to the highest ranked member of the PSM aggregation dataset.

At 3008A, probabilities may be computed for each PSM based on the expectation score recalculate in the merge feature 3006B, for example, using Peptideprophet (e.g., as described with reference to Keller. A., Nesvizhskii, A. I., Kolker, E. & Aebersold, R. Empirical statistical model to estimate the accuracy of peptide identifications made by MS/MS and database search. Anal. Chem. 74, 5383-5392 (2002)) and/or another suitable process. Optionally, a probability score indicative of match accuracy is computed for each PSM.

Optionally, the PSM aggregation dataset is divided into groups, for example, unmodified, standard search modification types, and other modification types. The division into groups may be using a threshold cutoff based on respective abundance in the PSM aggregation dataset. For each group, the PSM are sorted by probability score, and a threshold may be set for assuring false identification is below a selected FDR limit, for example, about 3%, 5%, 7%, or other value.

Optionally, the highest ranking PSMs are selected according to highest probability. When a difference in probability scores is below a defined percentage of the average probability score, the lower-ranked PSM are obtained and added to the modified sequence dataset. A certain PSM may be identified as the highest ranking PSM when the certain PSM is identified as having a highest probability score in one respective set of PSM and a lower ranked probability score in another respective set of PSM.

Optionally, spectra are annotated. Peaks are extracted from the PSM. For each peak, multiple theoretical fragment ions for an unmodified version of the respective peptide are computed. Each theoretical fragment ion is adjusted according to the modification mass shift. The respective peak is annotated with the theoretical fragment ions. Exemplary theoretical fragment ions include a, b, y precursor and/or diagnostic ions with potential ammonium and water lost in expected peptide charges.

Optionally, for each PSM, a searching for modification reporter ions is performed. A number of b and y ions are provided. A proportion of ion current (PIC) is computed. Unassigned peaks with significant intensity indicate a discrepancy between an observed spectrum defined by the respective spectra element of the plurality of PSMs and a matched peptide of the PSM.

In an exemplary implementation, the Philosopher package (e.g., as described with reference to Leprevost Felipe da Veiga, Haynes Sarah, N. A. Philosopher|A complete toolkit for shotgun proteomics data analysis. Nat. Methods doi:10.1038/s41592-020-0912-y) uses a target-decoy strategy to filter the data generating a combined PSM list for performing FDR calculations (e.g., psm.tsv). The FDR may be set to a suitable value, for example, about 3%, 5%, 7%, or other value, using a subgroup FDR threshold model where identified peptides were split into 3 groups: unmodified, highly abundant modifications and rare modifications. Alternative models for FDR correction may be used, such as for the case of PTM discovery, for example, as descried with reference to Devabhaktuni, A. et al. TagGraph reveals vast protein modification landscapes from large tandem mass spectrometry datasets. Nat. Biotechnol. 37, (2019), Fu. Y. & Qian, X. Transferred Subgroup False Discovery Rate for Rare Post-translational Modifications Detected by Mass Spectrometry <sup/>. Mol. Cell. Proteomics 13, 1359-1368 (2014), and/or n, Z. et al. PTMiner: Localization and quality control of protein modifications detected in an open search and its application to comprehensive post-translational modification characterization in human proteome. Mol. Cell. Proteomics 18, 391-405 (2019). For example, a global FDR may be performed without separating peptides into groups, which do not bias against rare modification types but increase false-positive rates. Alternatively or additionally, other decoy-independent models which avoid FDR entirely may be used, for example, as described with reference to Devabhaktuni, A. et al. TagGraph reveals vast protein modification landscapes from large tandem mass spectrometry datasets. Nat. Biotechnol. 37, (2019). In some embodiments, the choice for a highly stringent FDR increases confidence in the accuracy of identifications.

Optionally, for each spectrum assigned to a modified peptide, differences in scores (e.g., delta hyperscore) between the top-ranking peptide (with modification) and lower-ranked candidates are extracted from the dataset (e.g., psm file). For ambiguous matches, where the score differences are below about 3%, 5%, or 7%, or other value of the average score (e.g., delta score=1), the lower-ranked identifications (e.g., as documented in the MSFragger output files, pepXML) may be extracted. Those identifications are then considered as the potential hits for the following features of the process. Otherwise, only the leading match is used.

Optionally, the peak lists for each PSM is obtained, for example, from the MS raw file. A process, for example, CRUX (e.g., as described with reference to Park, C. Y., Klammer, A. A., Käli, L., MacCoss, M. J. & Noble, W. S. Rapid and accurate peptide identification from tandem mass spectra. J. Proteome Res. 7, 3022-3027 (2008)) version 3.1 or other suitable process, is used to create (e.g., all) possible theoretical fragment ions for the unmodified version of the peptide and adjust them according to the modification mass shift. The ion list may be much more comprehensive than what the matching process (e.g., MSFragger) uses, by optionally contains a, b, y, precursor, internal fragments and/or diagnostic ions with potential ammonium and water lost in all expected peptide charges. The list may then be used to annotate the spectrum peaks. A search for modification reporter ions (e.g., as described with reference to Kuster, B. ProteomeTools: Systematic characterization of 21 post-translational protein modifications by LC-MS/MS using synthetic peptides. (2018)) may be performed. For each PSM, the number of b and y ions may be reported and/or the proportion of ion current (PIC) may be calculated. Unassigned peaks with significant intensity may suggest a discrepancy between the observed spectrum and the matched peptide, and as such may be reported.

At 3008B, for each PTM of each PSM, a window of potential site positions may be created based on the annotated peaks. It is noted that the annotation may be performed in 3008A and/or in 3008B. Alternatively or additionally, site positions may be considered within the position window and/or alternative combination of modification with equivalent mass may be considered (e.g., two methyls are equivalent to a dimethyl, two glycine tails on two lysines are equivalent to a diglycine on one lysine). Potential site positions (e.g., all potential site positions) and/or alternative configurations may be reported, for example, presented on a display, and/or stored in an execution log file.

At 3008C, a search may be performed for identical masses and/or combination of masses that match the respective PTM mass shift indicative of mass decoy and/or isobaric masses. For each identified PTM an alternative solution may be considered by searching for identical masses and/or combination of masses that match the modification mass shift. For example, residues located before or after the identified peptide sequence may be identical in mass to predicted modification mass shifts and cause the matching process to falsely assign them as modifications at the peptide terminus instead of a longer peptide. Isobaric masses based on peptide amino acid sequence alone may be considered potential decoy and in most analysis, the PSM is filtered out as ambiguous. In response to finding the identical masses and/or combination of masses, the ambiguous respective identified PSM corresponding to the respective PTM may be removed from further consideration and/or further processing, i.e., are excluded from the PSM aggregation dataset.

Optionally, PSM with total peptide mass greater than average mass of a maximum peptide length plus a tolerance value are excluded from further consideration and/or further processing, i.e., are excluded from the PSM aggregation dataset. The exclude may be due to the technical problem of the search space having a defined limit for peptide length, which may result in incorrect assignments when a contaminant with a mass higher than max peptide is assigned to a peptide with a high mass shift modification. During the search for PTMs with large mass shifts (e.g., ubiquitin tail with 4 amino acid GGRL—383.228103 Da), this may lead to mis-assigned spectra. When the longer peptide is not part of the search space, a better match existing cannot be ruled out and/or that there is a higher scoring match above length limit cannot be ruled out. Therefore, potential mis-assignments may be filtered out by limiting the total peptide mass to the average mass of max peptide length plus 100 Da.

At 3008D, for each respective PSM, a dataset of known PSM (e.g., of healthy cells and/or cells with the target disease) may be search for a match to determine when the respective PTM site was reported before. Examples of known PSM databases include dbPTM (e.g., as described with reference to Huang, K.-Y. et al, dbPTM 2016: 10-year anniversary of a resource for post-translational modification of proteins. Nucleic Acids Res. 44, D435-D446 (2016)) and PhosphoSitePlus (e.g., as described with reference to Hornbeck, P. V. et al. PhosphoSilePlus, 2014: mutations, PTMs and recalibrations. Nucleic Acids Res. 43, D512-D520 (2015)) databases. Likelihood of the respective PSM being included in the modified sequence dataset is increased when the PSM is found in the dataset of known PSM.

At 3008E, the information collected in the prioritizing feature (e.g., 3008) may be integrated into a weighted score formula that ranks the identifications by their quality assessment. A threshold may be set to determine decoys modifications, which may be filtered out from the final identification list.

Optionally, one or two types of enrichment steps between samples may be implemented. In a rank base enrichment step, when a modified peptide is identified in rank 1 (e.g., top ranked) in at list one sample, any lower rank identification in other samples may be considered a valid hit. In a global FDR enrichment, when a modified peptide successfully passes the sub-group FDR threshold in one sample—any similar identification in other samples that pass the global FDR threshold will be considered a valid hit.

At 3010, modified sequences each including the PTM and sequences corresponding to the selected highest ranking PSMs, optionally after the prioritization process, are included in a modified sequence dataset. The modified sequence dataset stores an indication of binding motifs defined by identified PTM and corresponding sequence.

Optionally, the modified sequence dataset stores peptides selected from the group consisting of SEQ ID NO: 1-10746, 10817, 10819, 10820, 10823, 10824, 10826 and 10827, as described herein.

The modified sequence dataset is provided, for example, presented on a display, stored on a data storage device, forwarded to another device (e.g., server, storage), and/or provided to another process for further processing (e.g., to create the training dataset and/or for training the ML model as described herein).

The modified sequence dataset may be provided for selecting a certain binding motif having a certain PTM and corresponding amino acid sequence. The selected binding motif is capable of specifically binding an MHC (e.g. HLA I) presented peptide for treatment of the target disease.

Referring now back to FIG. 10, at 3102, the modified sequence dataset is received and/or generated. The modified sequence dataset may be generated, for example, as described with reference to FIG. 9.

At 3104, a training dataset may be created, by labelling each modified sequence of the modified sequence dataset with an indication of one or more of: an MHC type, parent gene, and position of the motif within a full protein length. Each modified sequence is for each respective motif of the modified sequence dataset. Each modified sequence including an amino acid sequence. PTM type, and position of the PTM on the amino acid sequence.

At 3106, training a machine learning model using the training dataset.

At 3108, the ML model is provided.

Optionally, for an input of a certain modified sequence defined by a combination of an amino acid sequence and at least one PTM that is fed into the trained ML model, an indication of whether the certain modified sequence is predicted to fit a binding motif that binds to a cell of the MHC type is obtained as an outcome of the ML model. Alternatively or additionally, for an input of an amino acid sequence of a full protein length and PTMs into the ML model, at least one modified sequence predicted to fit a binding motif is obtained as an outcome of the ML model.

Referring now back to FIG. 11, at 3202 the trained ML model is provided and/or generated.

At 3204, receiving an input is received, where the input is one or both of: (i) a certain modified sequence defined by an amino acid sequence and a PTM, and (ii) an amino acid sequence of a full protein length and PTMs.

At 3206, the input is fed into the trained ML model.

At 3208, an outcome of the ML model is obtained in response to the input. For the input of (i) a certain modified sequence defined by an amino acid sequence and a PTM, an outcome of an indication of whether the certain modified sequence is predicted to fit a motif that binds to a cell of the MHC type is obtained. For the input of (ii) an amino acid sequence of a full protein length and PTMs, an outcome of at least one motif predicted to be created from the full protein length and PTMs is obtained.

At 3210, the subject may be treated using the motif predicted to bind to a cell of the MHC type and/or the motif predicted to be created from the full protein length.

Various embodiments and aspects of the present invention as delineated hereinabove and as claimed in the claims section below find experimental and/or computational support in the following examples.

### EXAMPLES

Reference is now made to the following examples, which together with the above descriptions illustrate some embodiments of the invention in a non limiting fashion.

Inventors compared three different proteomics pipelines: 1) MaxQuant (e.g., as described with reference to Cox, J., Michalski, A. & Mann, M. Software Lock Mass by Two-Dimensional Minimization of Peptide Mass Errors. J. Am. Soc. Mass Spectrom. 22, 1373-1380 (2011)) version 1.6.0.16 2) MSFragger version 20180316+Philosopher version 20180924 3) And a pipeline based on embodiments described herein that implement MSFragger version 20180316 and Philosopher version 20180924.

For a search including phosphorylation site on S, T, or Y of endogenous peptides (search space of ˜31 billion potential peptides). MaxQuant arrived at search results within a week while the pipeline based on embodiments described herein produced its result in ˜2 hours.

Table 1 below presents results of the computational experiment comparing different computational process to the parallel processor based computational process described herein, in accordance with some embodiments of the present invention. Where:
(1) (2) denote Cell line HEK293, 3 replicas are without treatment, 3 replicas were stimulated with INF+TNF, for more information see Wolf-Levy. H. et al. Revealing the cellular degradome by mass spectrometry analysis of proteasome-cleaved peptides. Nat. Biotechnol. (2018), doi:10.1038/nbt.4279.
(3) denotes Multiple cancer cell lines HLA class I data, taken from Bassani-Sternberg, M., Pletscher-Frankild, S., Jensen, L. J. & Mann. M. Mass Spectrometry of Human Leukocyte Antigen Class I Peptidomes Reveals Strong Effects of Protein Abundance and Turnover on Antigen Presentation. Mol. Cell. Proteomics 14, 658-673 (2015).
(4) denotes that as reference data, the SwissProt database from UniProtKB, downloaded on the 19 Sep. 2018 without isoform (20,394 sequences), Contaminate data taken from MaxQuant version 1.6.0.16 with additional three entries for protein G and mAb that the MAPP protocol uses (248 sequences)
(5) denotes MaxQuant run on window server, 64-bit OS, with Intel Xeon CPU E5-2699 v4 @ 2.20 GHz (6 processors) with 64 GB RAM
(6) denotes MSFragger+Philosopher run on Linux system: HP type C, 896 GPU cores. GBU: Tesla 52050.

As used herein the term “about” refers to ±10%.

The terms “comprises”. “comprising”. “includes”, “including”. “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”. “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”. John Wiley & Sons, New York (1988); Watson et al., “Recombinant DNA”, Scientific American Books, New York; Birren et al. (eds) “Genome Analysis: A Laboratory Manual Series”. Vols. 1-4, Cold Spring Harbor Laboratory Press. New York (1998); methodologies as set forth in U.S. Pat. Nos. 4,666,828; 4,683,202; 4,801,531; 5,192,659 and 5,272,057; “Cell Biology: A Laboratory Handbook”. Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition). Appleton & Lange. Norwalk, C T (1994); Mishell and Shiigi (eds). “Selected Methods in Cellular Immunology”. W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames. B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press. (1986); “A Practical Guide to Molecular Cloning” Perbal. B., (1984) and “Methods in Enzymology” Vol. 1-317, Academic Press; “PCR Protocols: A Guide To Methods And Applications”, Academic Press. San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

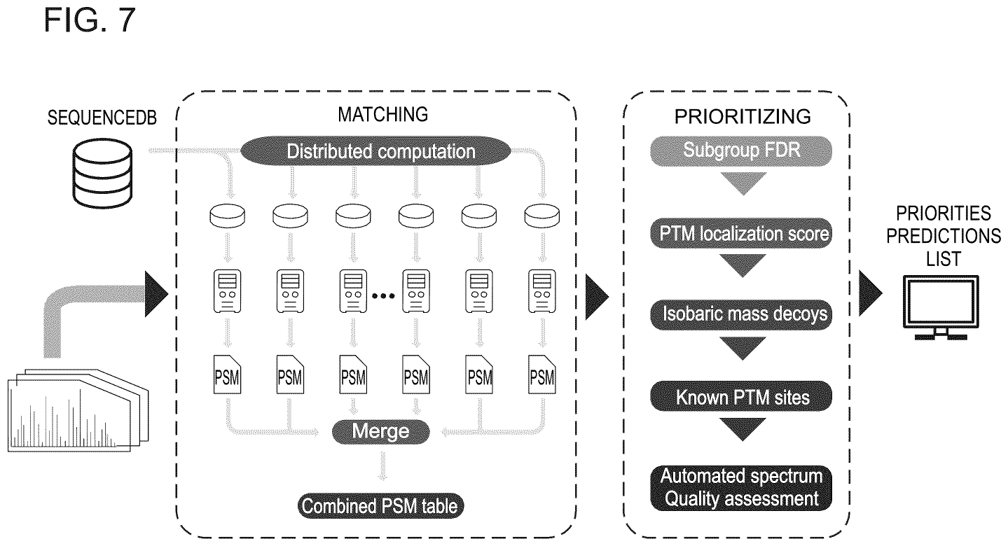

PROtein Modification Integrated Search Engine (PROMISE)—To overcome the challenges of searching for post translational modifications (PTMs) on endogenous peptides in a systematic manner and optimize search efficiency, the present inventors have developed a PROtein Modification Integrated Search Engine (PROMISE). Specifically, this computational pipeline (FIG. 7) was developed to improve spectral assignment rates in mass spectrometry (MS) data of endogenous peptides. This was accomplished by including proteoforms with PTMs in the peptide search space. PROMISE has two stages: a) a matching phase and b) a prioritizing phase (supplementary pipeline documentation). The matching phase reduces the algorithm running time, utilizing the ultrafast MSFragger³⁷ software and parallel computing on a CPU cluster. The prioritizing phase includes several computational steps to distinguish between true and false hits, validate PTM identifications and site position and rank predictions by their biological relevance and antigenic potential. The pipeline was coded in Python 2.7.

Matching phase—The program accepts MS raw files (mzML format), proteome reference sequence file (fasta format) and a list of variable modifications (amino acid and the expected mass shift) as inputs. A single search task can be distributed into thousands of MSFragger [Andy T. et al. MSFragger: ultrafast and comprehensive peptide identification in shotgun proteomics. 14, 39-46 (2017)] instances that are performed in parallel on a CPU cluster. The search tasks are split by dividing the search into batches and the list of variable modifications into each potential combination up to 7 mass shifts per instance. A merge program then assembles the different output results, prioritizing the best peptide to spectra match (PSM) solution according to its hyperseore and minimum delta masses. It also recalculates the statistical parameters needed for further FDR calculation.

Prioritization phase—The pipeline uses Peptideprophet [Keller, A., et al. Anal. Chem. 74, 5383-5392 (2002)] to compute probabilities for each PSM. The Philosopher package (www(dot)philosopher(dot)nesvilab(dot)org/) uses a target-decoy strategy to filter the data generating a combined PSM list (psm.tsv). For the analysis presented hereinbelow, a subgroup FDR whereby the identifications was split into three groups was used: unmodified, standard search modification types (n-acetylation and methionine oxidation) and the other modification types. Cutoff was set to 5%. In cases where subgroup FDR was used across multiple cohorts, any peptide that passed the subgroup FDR in at least one cohort was included. Alternative models exist for FDR correction, specifically in the case of PTM discovery [Devabhaktuni. A. et al. Nat. Biotechnol. 37, 469-479 (2019); Fu, Y. & Qian, X. Mol. Cell. Proteomics 13, 1359-1368 (2014); An, Z. et al. Mol. Cell. Proteomics 18, 391-405 (2019)]. For example, one can perform a global FDR without separating peptides into groups, which do not bias against rare modification types but increases false positive rates. Likewise, there are newer decoy-independent models which avoid FDR entirely [Devabhaktuni. A. et al. Nat. Biotechnol. 37, 469-479 (2019)]. Here the choice for a highly stringent FDR increases confidence in the accuracy of identifications.

For each spectrum assigned to a modified peptide, differences in scores (delta hyperscore) between the top-ranking peptide (with modification) and lower-ranked candidates are extracted from the psm file. For ambiguous matches, where the score differences are below 5% of the average score (delta score=1), the program retrieves the lower-ranked identifications as documented in the MSFragger output files (pepXML). Those identifications are then considered as the potential hits for the following steps of analysis. Otherwise, only the leading match is used.

Spectrum annotation: The program retrieves the peak lists for each PSM from the MS raw file. It uses CRUX [Park, C. Y., et al. J. Proteome Res. 7, 3022-3027 (2008)] version 3.1 to create all possible theoretical fragment ions for the unmodified version of the peptide and adjust them according to the modification mass shift. The ion list is much more comprehensive than what MSFragger uses in its matching algorithm and contains a, b, y, precursor and diagnostic ions with potential ammonium and water lost in all expected peptide charges. The list is then used to annotate the spectrum peaks. The program also searches for modification reporter ions [Kuster, B. ProteomeTools: Systematic characterization of 21 post-translational protein modifications by LC-MS/MS using synthetic peptides. (2018)]. For each PSM, the number of b and y ions will be reported and the proportion of ion current (PIC) is calculated. Unassigned peaks with significant intensity suggest a discrepancy between the observed spectrum and the matched peptide, and as such will be reported.

PTM localization: For each modification, a window of potential site positions is created based on the annotated peaks from the previous step. Alternative site positions are considered within the position window and alternative combination of modification with equivalent mass are also considered (e.g. two methyls are equivalent to a dimethyl, two glycine tails on two lysines are equivalent to a diglycine on one lysine). All potential site positions and alternative configurations are reported.

Search for mass decoys or isobaric masses: For each identified PTM an alternative solution is considered by searching for identical masses or combination of masses that match the modification mass shift. For example, residues located before or after the identified peptide sequence can be identical in mass to predicted modification mass shifts and cause the matching algorithm to falsely assign them as modifications at the peptide terminus instead of a longer peptide. Isobaric masses based on peptide amino acid sequence alone are considered potential decoy and in most analysis, the PSM will be filtered out as ambiguous.

Known site search: The program scans dbPTM [Huang, K.-Y. et al. Nucleic Acids Res. 44, D435-D446 (2016)] and PhosphoSitePlus [Hornbeck. P. V. et al. Nucleic Acids Res. 43, D512-D520 (2015)] databases to determine if the PTM site was reported before. The results of the search are documented in the final output report.

Performance—To evaluate pipeline performance, the full human proteome from UniProtKB was used as reference data and endogenous proteasome-cleaved peptides⁶⁰ (length between 6 and 40 amino acids) with 5 variable modifications were searched for, creating a search space of ˜31 billion potential peptides. In a comparison of PROMISE to MaxQuant³⁸ (see table 1 hereinbelow), it was found that the former reached results in around two hours (1:55 hours) while MaxQuant produced its result in around a week (169:50 hours). To assess the reproducibility of the identified peptides by the distributed version and the standalone one the spectral assignments from identical sets of data were compared, indicating that 99.2% were identical.

TABLE 1
PROMISE pipeline performance comparison to MSFragger and MaxQuant

| | | | | | | | | | MSFragger + | | | |
|---|---|---|---|---|---|---|---|---|---|---|---|---|
| | | | | | | | | | Philosopher | | | |
| | | | | | | | MaxQuant | | Standalone | | PROMISE | |
| | | | | | | Theoretical | | PSM | | PSM | | PSM |
| | | | | | | peptides | | Peptides | | Peptides | | Peptides |
| | | | | | | (search | Running | Proteins | Running | Proteins | Running | Proteins |
| | | | | | Peptide | space) | time | (FDR = | time | (FDR = | time | (FDR = |
| | digestion | Data | MS/MS | Modification | length | (Millions) ⁽⁴⁾ | (H) ⁽⁵⁾ | 0.01) | (H) ⁽⁶⁾ | 0.01) | (H) ⁽⁶⁾ | 0.01) |
| 1 | tryptic | HEK293 ⁽²⁾ | 276,149 | Oxidized | 6-40 | 4.44 | 3:10 | 156,723 | 0:58 | 162,137 | 0:48 | 161,231 |
| | | | | Methionine + N | | | | (57%) | | (59%) | | (58%) |
| | | | | terminus | | | | 32,801 | | 30,931 | | 31,134 |
| | | | | acetylation | | | | 4,967 | | 5,290 | | 5,286 |
| 2 | tryptic | HEK293 ⁽²⁾ | 276,149 | Oxidized | 6-40 | 81.23 | 7:18 | 156,772 | 1:15 | 161,314 | 1:10 | 159,897 |
| | | | | Methionine + N | | | | (57%) | | (58%) | | (58%) |
| | | | | terminus | | | | 32,779 | | 30,748 | | 30,959 |
| | | | | acetylation + | | | | 4,921 | | 5289 | | 5273 |
| | | | | phosphorylation | | | | | | | | |
| | | | | on STY | | | | | | | | |
| 3 | Non- | MAPP- | 137,241 | Oxidized | 6-40 | 1,219.28 | 20:09 | 29,632 | Not tested | | 0:18 | 31,476 |
| | specific | HEK293 ⁽¹⁾ | | Methionine + N | | | | (22%) | | | | (23%) |
| | | | | terminus | | | | 7472 | | | | 9068 |
| | | | | acetylation | | | | 1286 | | | | 1503 |
| 4 | Non- | MAPP- | 137,241 | Oxidized | 6-40 | 31,101.01 | 169:50 | 28,096 | Fail (to many | | 1:55 | 33,273 |
| | specific | HEK293 ⁽¹⁾ | | Methionine + N | | | | (20%) | theoretical peptides) | | | (24%) |
| | | | | terminus | | | | 7125 | | | | 7574 |
| | | | | acetylation + | | | | 1233 | | | | 1183 |
| | | | | phosphorylation | | | | | | | | |
| | | | | on STY | | | | | | | | |
| 5 | Non- | HLA- | 1,081,814 | Oxidized | 8-15 | 213.38 | 9:28 | 76,125 | Not tested | | 0:49 | 176,107 |
| | specific | multi | | Methionine + N | | | | (7%) | | | | (16%) |
| | | cell | | terminus | | | | 24,250 | | | | 37,679 |
| | | lines ⁽³⁾ | | acetylation | | | | 8,142 | | | | 10,060 |
| 6 | Non- | HLA- | 1,081,814 | Full list-27 | 8-15 | ~1,000,000 | Not practical | | Fail | | ~48:00 | 142,591 |
| | specific | multi | | modifications () | | | | | | | | (13%) |
| | | cell | | | | | | | | | | 29,586 |
| | | lines ⁽³⁾ | | | | | | | | | | 9615 |

^{(1) (2)} Cell line HEK293, 3 replicas are without treatment, 3 replicas were stimulated with INF + TNF, for more information see Ref ¹⁴
⁽³⁾ Multiple cancer cell lines HLA class I data, taken from Bassani et al ¹⁵
⁽⁴⁾ As reference data, the SwissProt database from UniProtKB, downloaded on the 19 Sep. 2018 without isoform (20,394 sequences), Contaminate data taken from MaxQuant version 1.6.0.16 with additional three entries for protein G and mAb that the MAPP protocol uses (248 sequences)
⁽⁵⁾ MaxQuant run on window server, 64-bit OS, with Intel Xeon CPU E5-2699 v4 @ 2.20 GHz (6 processors) with 64 GB RAM
⁽⁶⁾ MSFragger + Philosopher run on Linux system: HP type C, 896 GPU cores, GBU: Tesla S2050

Modification Annotation and Classification—In order to assess the effects of modifications in a holistic manner, modifications that may arise during sample processing (“experimental”) were differentiated from biological modifications that reflect the cellular state (“biological”). This was effected using the UNIMOD classification system (unimod.org) which defines modifications as post-translational or multiple (here termed “biological”) or artifact (here termed “experimental”). Including experimental modifications in the search allowed matching spectra to a presented peptide that would otherwise have remained unassigned. However, some of the types of modifications that were termed as experimental also occur biologically. Because they are chemically identical they cannot be distinguished, the present inventors consider that peptides identified with an experimental PTM may exist in the cell in either their modified or unmodified form. Therefore, both the experimental and biological types of modifications were include in the analysis for maximum enrichment of immunopeptide identification. When a peptide contains multiple modification types, a leading modification was defined, prioritizing biological modifications over experimental ones.

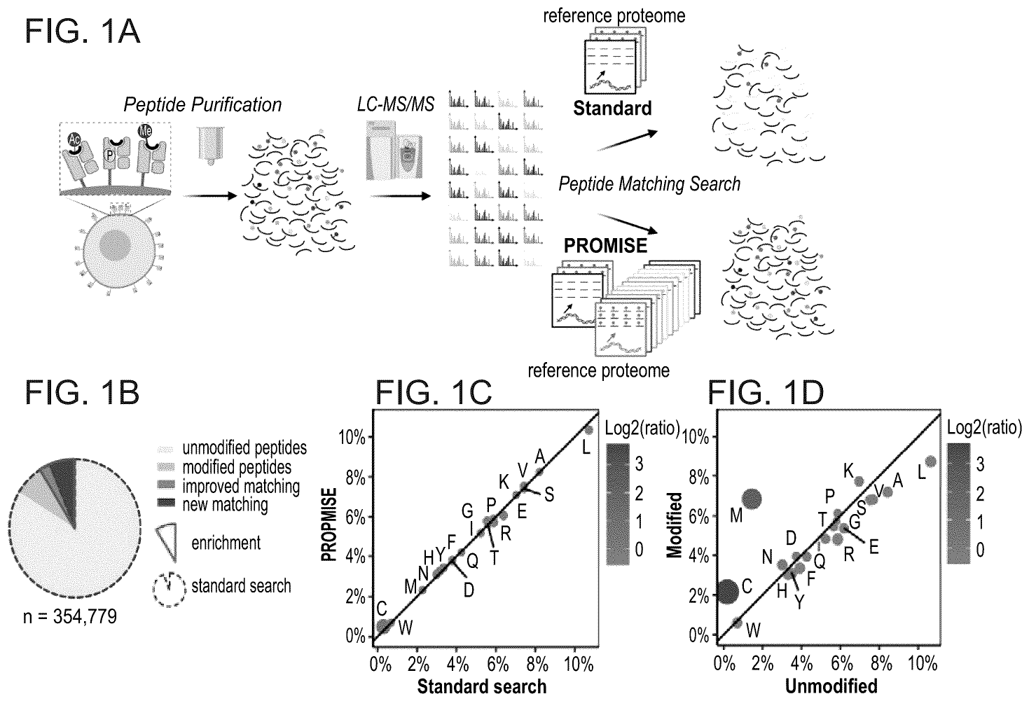

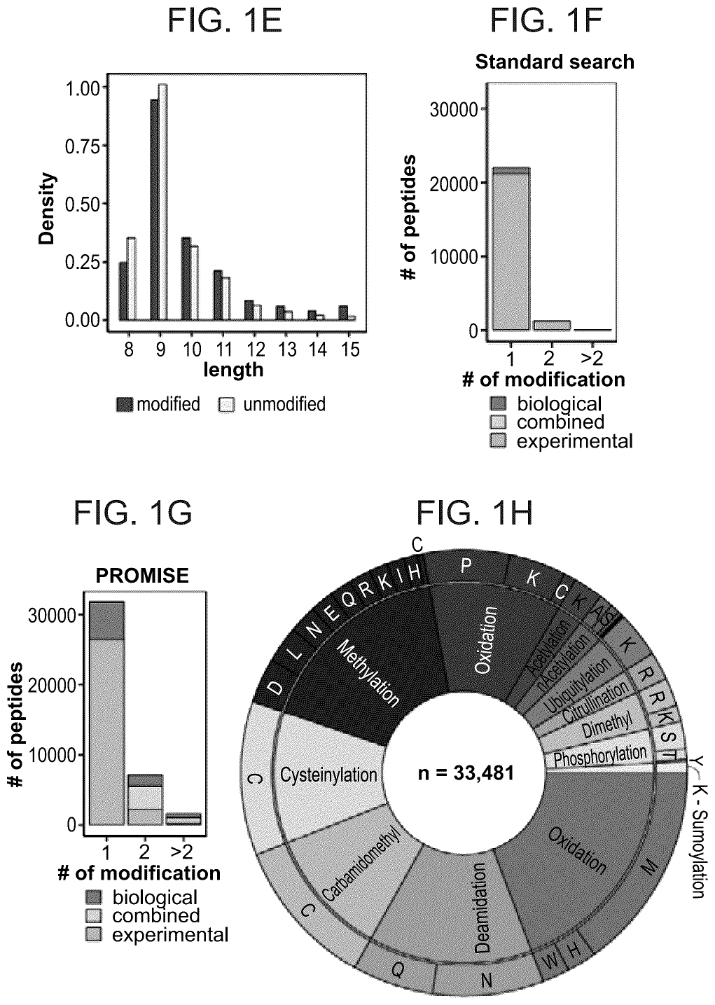

Search mass boundary effect correction—The search space in the analysis is bounded by a 15 amino acid peptide length. This can result in incorrect assignments when a contaminant with a mass higher than 15 AA is assigned to a 15-mer peptide with a high mass shift modification. As we search for PTMs with large mass shifts (e.g. ubiquitin tail with 4 amino acid GGRL—383.228103 Da), this can lead to missasigned spectra. Because the longer peptide is not part of our search space we cannot rule out that a better match exists or that there is a higher scoring match above 15 AA. Therefore, to avoid a bias we filter out potential mis-assignments by limiting the total peptide mass to the average mass of 15 amino acid peptide plus 100 Da when comparing peptide lengths (FIG. 1E).

HLA motif—HLA I motif presentation was designed to capture both the main anchor position 2 and C-terminus and the TCR recognition area (position 3-7). The presented motif was created by collecting all the epitopes reported for the specific HLA haplotype from the IEDB 4 database. Epitopes with length less than 8 amino acids were discarded. To correct for discrepancies in length, the motif was constructed from positions 1 to 7 starting from the N terminus followed by the C terminus and its preceding position. For 9 mer epitopes, the motif is taken from all 9 positions, for 8-mer epitopes the 7^{th} position is duplicated and presented as both positions 7 and 8/C-1. For epitopes longer than 9 residues, the motif skips positions 8 till C-terminus-1. Motif logos were plotted using Seq2Logo 2.0⁶¹ with default parameters. The comparable motif was created using Two-Sample-Lo⁶².

Site score—The score was designed to determine if a PTM tends to fall within the peptide anchor positions or the center positions (3-7) of the peptide; by summing up the differences between the distribution values of modified amino acids vs. the background in the anchor positions (2, C-terminus) and subtracting the sum of distribution differences in the center positions (3-7). In this manner, an enrichment in the anchor positions will result in a high positive score while enrichment in the center of the peptide will result in a negative score. In case both the center and anchor positions are enriched or under-represented, the score will be close to zero and the modification tendency cannot be classified to be in a specific area.

Modeling the Peptide-Receptor Complex—

General modeling scheme—The FlexPepBind scheme used^{63,64} allows the structure-based evaluation of the relative binding affinities of different peptides for a given receptor, using a solved structure of a representative peptide-protein interaction as template. Structures of peptide-MHC complexes were generated by “threading” candidate peptide sequences onto this template, followed by refinement using Rosetta FlexPepDock⁵⁰. The top-scoring models were selected to discriminate stronger from weaker binders and inspected for the structural details of an interaction.

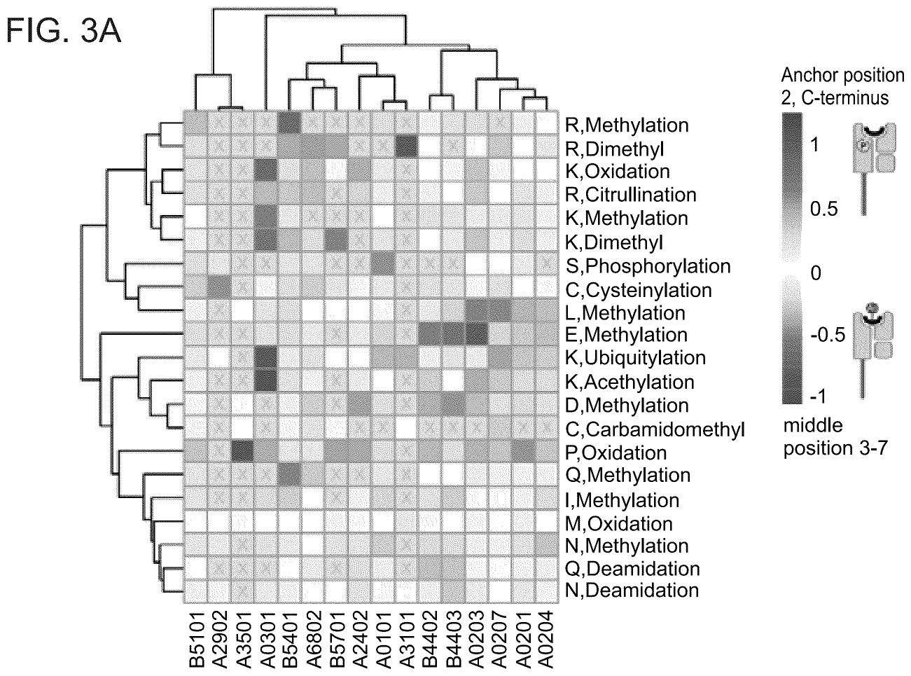

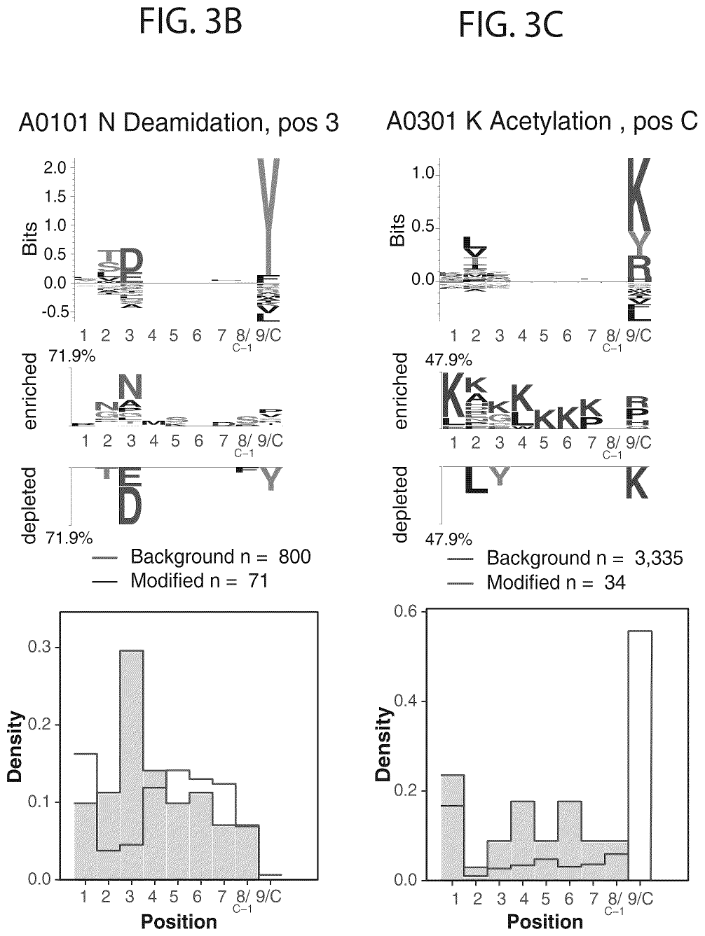

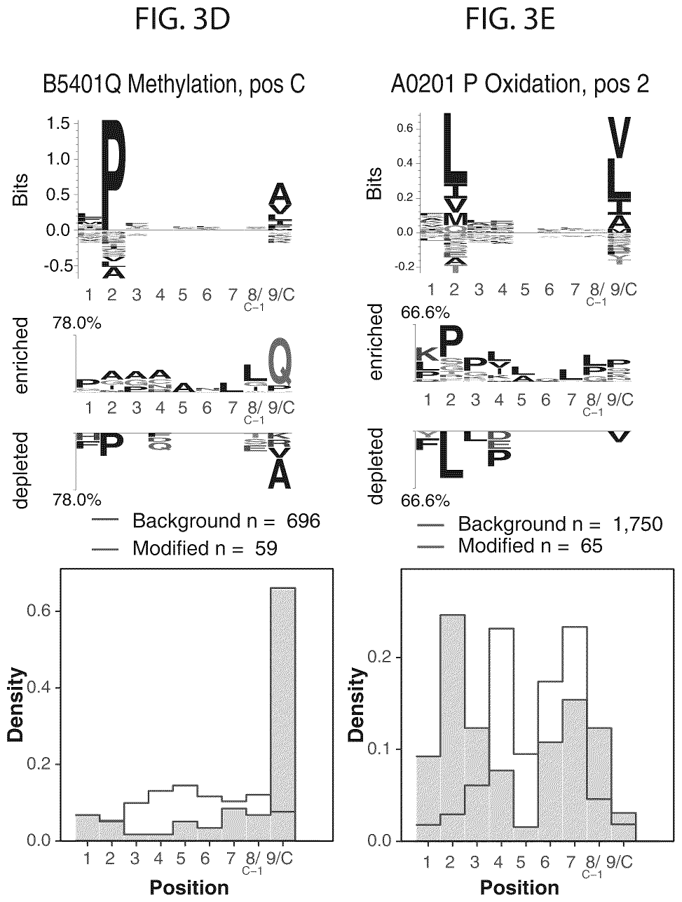

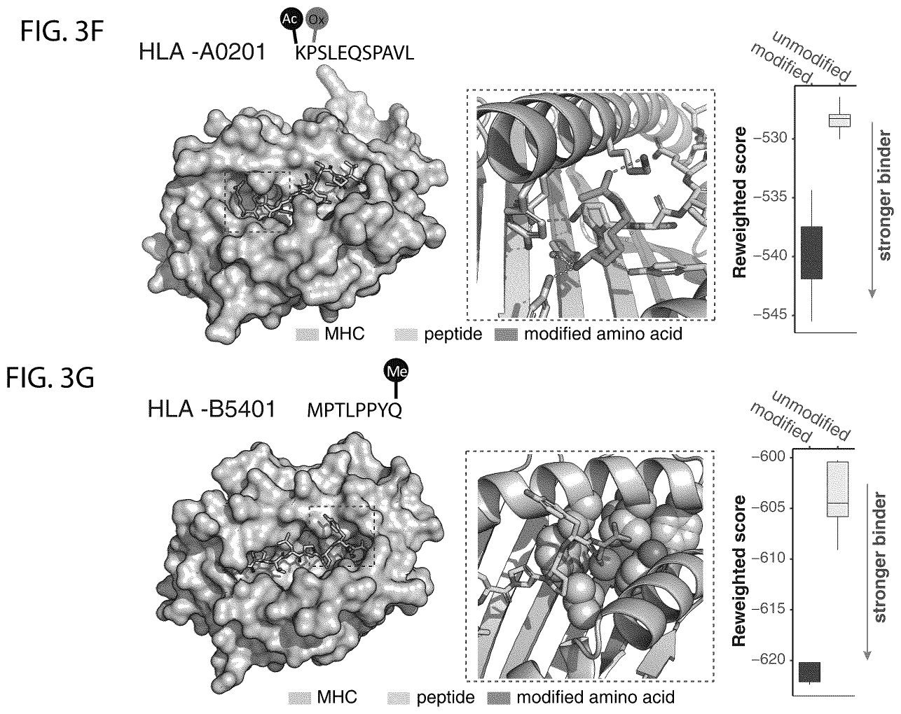

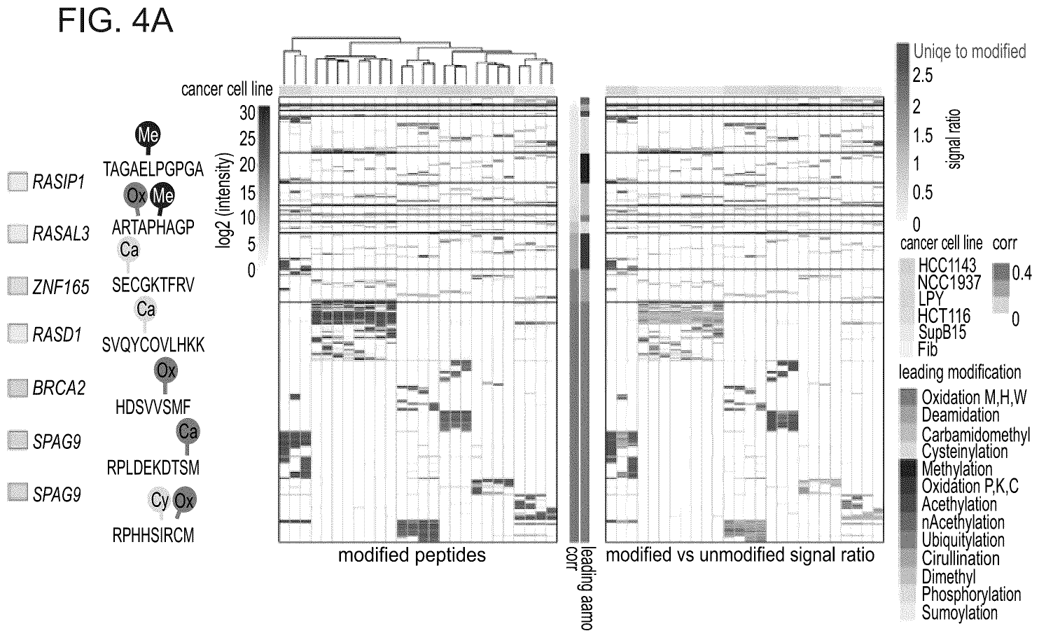

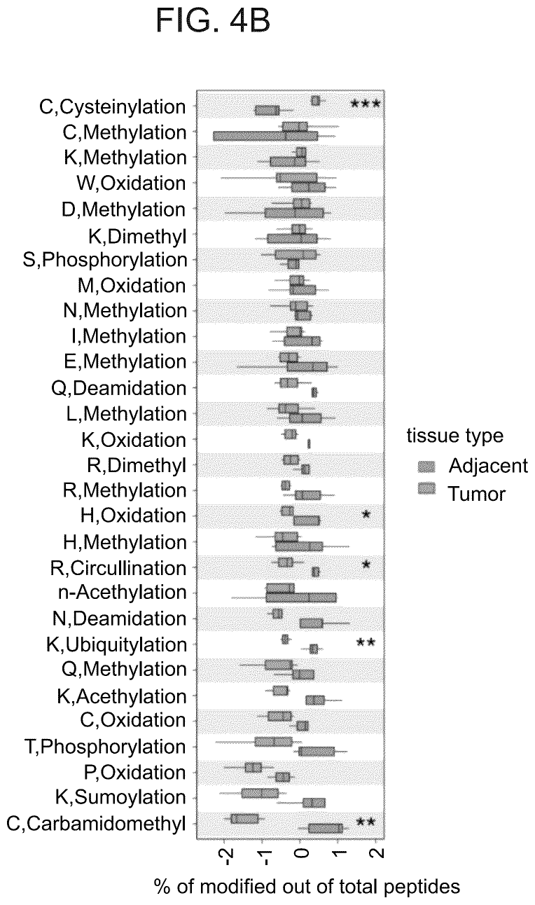

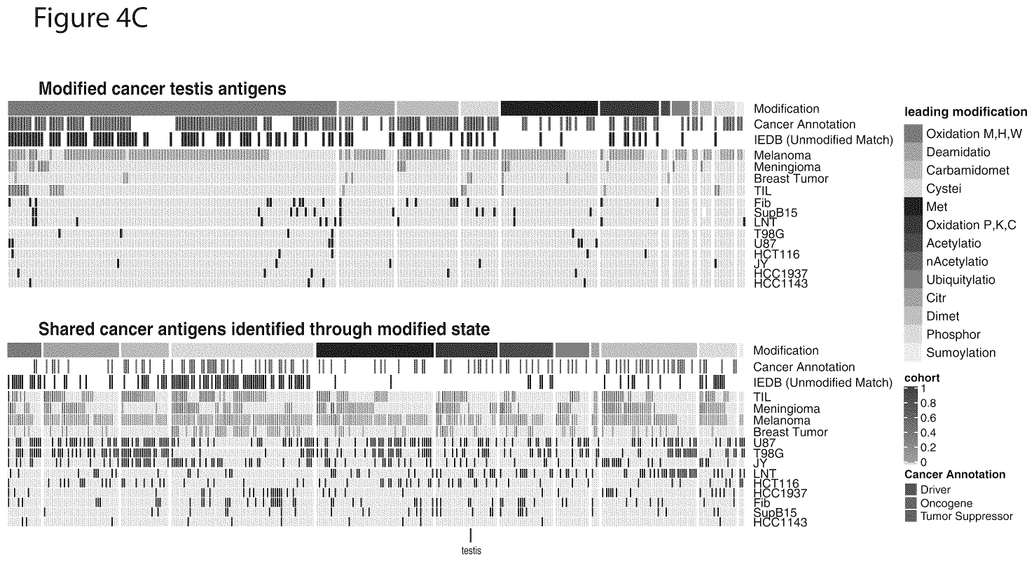

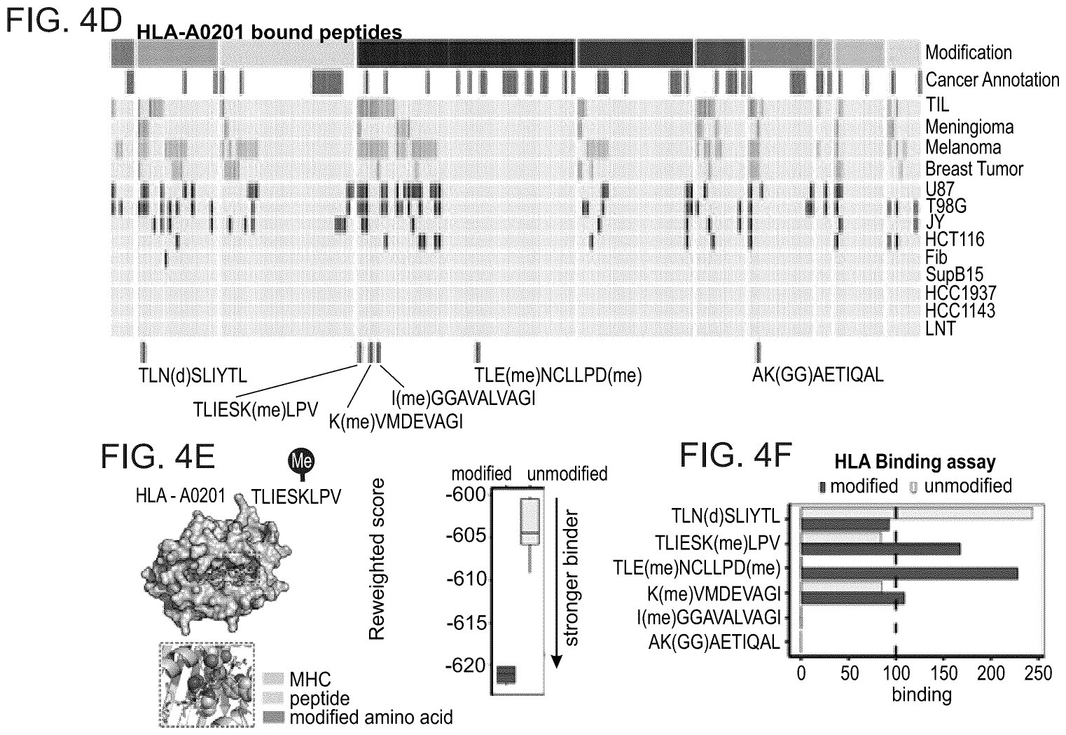

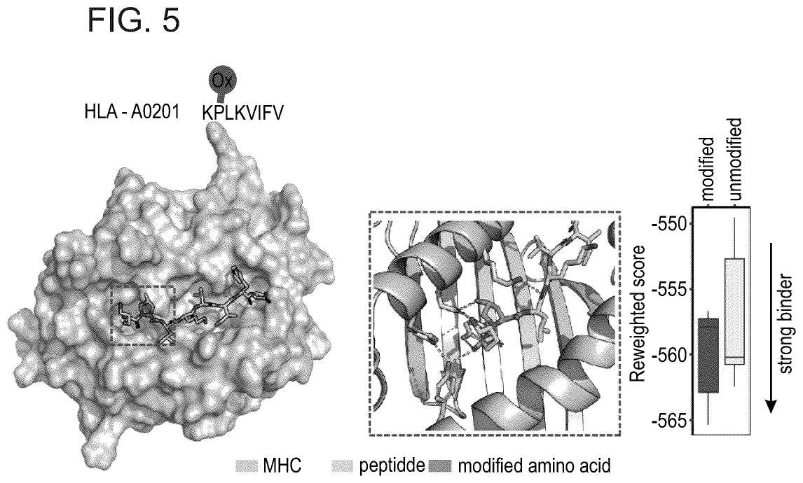

Selection of templates for modeling—For each of the MHC alleles (receptors) and peptides, different available PDB structures we evaluated to serve as templates for the modeling of the structure and relative binding affinities of different peptides. Screening for relevant PDB templates was guided by 3 main requirements: (1) matching MHC allele, (2) matching peptide length, and (3) similarity of peptide anchor residues. Specifically, for peptide K(ac)P(ox)SLEQSPAVL (SEQ ID NO: 10817 having the recited modifications) bound to HLA-A02 (FIG. 3F) PDB id 5D9S⁶⁵ [HLA-A02 bound to FVLELEPEWTV (SEQ ID NO: 10828)] was used; for peptide KP(ox)LKVIFV (SEQ ID NO: 10827 having the recited modification) bound to HLA-A02 (FIG. 5), the peptide backbone from PDB id 4F7T⁶⁶ [HLA-A24 bound to RYGFVANF (SEQ ID NO: 10829)] and the same MHC receptor structure (from PDB id 5D9S) were used; for peptide MPTLPPYQ(me) (SEQ ID NO: 10818 having the recited modification) bound to HLA-B54 (FIG. 3G), PDB id 3BWA⁶⁷ [HLA-B35 bound to FPTKDVAL (SEQ ID NO: 10830)] was used. Residues that differ between the MHC alleles were “mutated” using the fix backbone protocol (Rosetta fix_bb; [8]); for peptide TLIESK(me)LPV (SEQ ID NO: 10823 having the recited modification) bound to HLA-A02 (FIG. 4F), PDB id 3MRK [HLA-A02 bound to PLFQVPEPV (SEQ ID NO: 10831)] was used.

Modeling peptide onto MHC receptor using the selected template—Using the Rosetta fixbb protocol for fixed backbone design⁶⁸, the desired peptide sequence was modeled onto the template peptide, while keeping the side chains of the receptor fixed. Following, Rosetta FlexPepDock refinement in full-atom mode was used to optimize the structure of the complex with the threaded target peptide (all peptide atoms, as well as the receptor interface sidechains were allowed to move). For each sequence, 200 models were generated. These were scored, and the 5 top-models were selected to represent the MHC-peptide interaction of interest. Comparison of the top scoring models of the modified peptides and corresponding non-modified peptides allowed inspection of the atomic details of their differential binding.

Scoring function—The standard Rosetta score function was used, and models were assessed according to their FlexPepDock reweighted score (sum of Total score, Interface score and Peptide score; where Total score is the overall Rosetta energy score for the complex. Interface score is the energy of pair-wise interactions across the peptide-protein interface and Peptide score is the sum of the Rosetta energy function over the peptide residues). This score was shown to discriminate well near-native structures in previous FlexPepDock modeling studies⁷⁰.

MSFragger search parameters—Search parameters were set to default for close search with the following changes: Precursor true tolerance was set to 10 ppm; fragment mass tolerance was set to 20 ppm. Search enzyme was set to nonspecific enzyme with cleavage after ARNDCQEGHILKMFPSTWYV (SEQ ID NO: 10832). Peptide lengths were set between 8 and 15. Num enzyme termini=0, clip nTerm M=1, allow multiple variable mods on residue=0, max variable mods per mod=3, max variable mods combinations=65000.

ProImmune binding assay—ProImmune (www(dot)proimmune(dot)com) Module 2 REVEAL Binding Assay measure the yield of correctly conformed MHC-peptide complex following incubation of the recombinant MHC allele and peptide of interest using a conformational-dependent antibody in an immunoassay. Each peptide is given a score relative to the positive control peptide, which is a known T cell epitope.

Bioinformatics and data analysis—Statistical analyses were performed in R v 3.6.1. heatmap was drawn with pheatmap 1.0.12 and ComplexHeatmap 2.2.0 R package with Euclidean distances for clustering where relevant. Experimental schematics were generated using BioRender.

### Example 1

### Identification of PTMS on HLA I-Bound Peptides Using a Novel Protein Modification Integrated Search Engine

Establishment of a novel PROtein Modification Integrated Search Engine (PROMISE)—Current proteomics software focuses on data from samples where an exogenous enzyme, like trypsin, was used to digest the proteins into peptides. This reduces the potential search space to only peptides with either lysine (K) or arginine (R) terminal residues. By contrast, HLA class I peptides are cleaved by the proteasome and a number of endopeptidases, generating peptides that are between 8 and 15 amino acid residues and with any potential terminal residue. Computationally, this means that the search space for endogenously-cleaved peptides with modifications must contain every potential protein fragment with multiple potential mass shifts, leading to an exponential growth of the search space and making search times impractical³⁶. To overcome the challenges of searching for post translational modifications (PTMs) on endogenous peptides in a systematic manner, the present inventors developed a PROtein Modification Integrated Search Engine (PROMISE). PROMISE utilizes distributed computing with an adapted version of MSFragger³⁷ to enable efficient search against combinatorial reference data with multiple modifications. To evaluate pipeline performance PROMISE was compared to MaxQuant³⁸ showing a 100-fold decrease in search time (Table 1 hereinabove). Further, results obtained by PROMISE and standalone MSFragger were 99.2% identical, confirming that the distributed computing has not affected peptide identification. In the next step PROMISE was applied to search for multiple types of PTMs on HLA I-bound peptides, looking for insight into PTM-driven antigenicity.

Analysis by PROMISE increases identification of modified peptides, enriching the identified immunopeptidome by 11%—To identify a broad range of PTMs, 29 modification combinations of 12 modification types (36 mass shifts; Table 2 hereinbelow) were defined as a variable modification on 16 different amino acids and protein termini (termed hereafter ‘multi-modification search’). These include biological modifications such as methylation, acetylation, phosphorylation, citrullination, ubiquitination, and sumoylation along with multiple technical modifications such as oxidation, deamidation, carbamidomethylation and cysteinylation. Subsequently. PROMISE (FIG. 1A) was used to analyze previously published high-resolution HLA immunopeptidomics data^{11,18,19,39,40} of patient tumors tissues (35), healthy adjacent tissue (5), cancer cell lines (13) and TILs (2). To identify peptides for which the modified state was a better match to the spectrum, the results were compared to the original search criteria, which only included methionine oxidation and protein N-terminus acetylation (termed hereafter ‘standard search’). In both cases, a subgroup FDR at 5% was used by splitting spectra into three different groups based on modification state, ensuring identifications was not increased merely by altering the false positive rate. The multi-modification search identified 32,798 modified peptides, 12.228 of the peptides identified were unique to the multi-modification search, thereby enriching the pool of immunopeptides identified (data not shown).

Out of the peptide to spectrum matches (PSMs) which conflicted between the two searches (1.34% of PSMs; 10,019 peptides), 86% received a higher scoring match in the multi-modification search. On average, the match score was increased by 15%, suggesting the inclusion of a modification in the predicted peptide better described the spectrum, and the unmodified peptide assignment was a false identification. In total, 10.94% of the peptides identified were unique to the multi-modification search, thereby enriching the pool of immunopeptides identified (FIG. 1B).

While the amino acid composition of the immunopeptidome was similar between the standard search and PROMISE, an enrichment in amino acids that carry modifications were observed when comparing the modified and unmodified peptide subsets (FIGS. 1C-D). For example, as previously described³⁵, cysteines are consistently under-represented in immunopeptidomics analyses, yet constitute 2% of the modified immunopeptidome. When comparing the distribution of peptide lengths between the modified and unmodified peptides a shift towards longer peptides was observed in the modified subset [p value=2.2e-16; Wilcoxon](FIG. 1E). The UNIMOD database classification was used to differentiate between two general types of modifications: modifications that may arise during sample processing (“technical”) and modifications that reflect the cellular state (“biological”). PROMISE increased the identification of modified peptides, in particular those with biological modifications (FIGS. 1F-G). In addition, identification of peptides with two or more modification was increased six-fold as compared to a standard search (FIGS. 1F-G). In total, 19.630 modification sites were identified that were unique to PROMISE, 88% of which were not included in a standard search (FIG. 1H).

TABLE 2
List of PTMs

| Modification | UNIMOD | Mass | | UNIMOD | |
|---|---|---|---|---|---|
| name | Accession # | shift | Amino acid | classification | remark |
| Methionine | 35 | 15.99490 | M | Artefact | Common |
| oxidation | | | | | chemical non- |
| | | | | | enzymatic |
| | | | | | modification. |
| | | | | | Appears in |
| | | | | | most MS |
| | | | | | searches ⁷² |
| protein N- | 1 | 42.01060 | [X]@N- | Multiple | |
| termini | | | terminus | | |
| acetylation | | | | | |
| Phosphorylation | 21 | 79.96633 | YTS | PTM | |
| Acetylation | 1 | 42.01060 | K | Multiple | ⁷³ |
| Methylation | 34 | 14.01565 | C, H, N, Q, K, R, I, | PTM | |
| | | | L, D, E | | |
| Di-methylation | 36 | 28.0312 | K, R | PTM | |
| Oxidation | 35 | 15.99490 | W, H, K, P, C | KPC-PTM | ⁷⁴ |
| | | | | WH- | |
| | | | | Artefact | |
| Deamidation | 7 | 0.98402 | NQ | Artefact | NQ-Artefact |
| Citrullination | 7 | 0.98402 | R | PTM | Enzymatic |
| | | | | | modification |
| Ubiquitination | 1263 | 57.0215 | K | Other | |
| | 121 | (G) | | Other | |
| | 535 | 114.0429 | | Chemical | |
| | | (GG) | | derivative | |
| | | 270.144 | | | |
| | | (GGR) | | | |
| | | 383.228103 | | | |
| | | (GGRL) | | | |
| Sumoylation | 1293 | 215.0906 | K | Other | G and GG- |
| | | (GGT) | | | cannot |
| | | 343.149184 | | | distinguish |
| | | (GGTQ) | | | between |
| | | | | | ubiquitin, |
| | | | | | Sumo or |
| | | | | | FAT10 |
| FAT10 | 1990 | 227.127 | K | PTM | |
| | | (GGI) | | | |
| | | 330.136176 | | | |
| | | (GGIC) | | | |
| Cysteinylation | 312 | 119.004099 | C | Multiple | |
| Carbamidomethyl | 4 | 57.021464 | C | Chemical | Artefact-used |
| | | | | derivative | as fix |
| | | | | | modification |
| | | | | | in trypsin |
| | | | | | digestion |

### Example 2

### Characterization of the Identified Modified HLA I-Bound Peptides

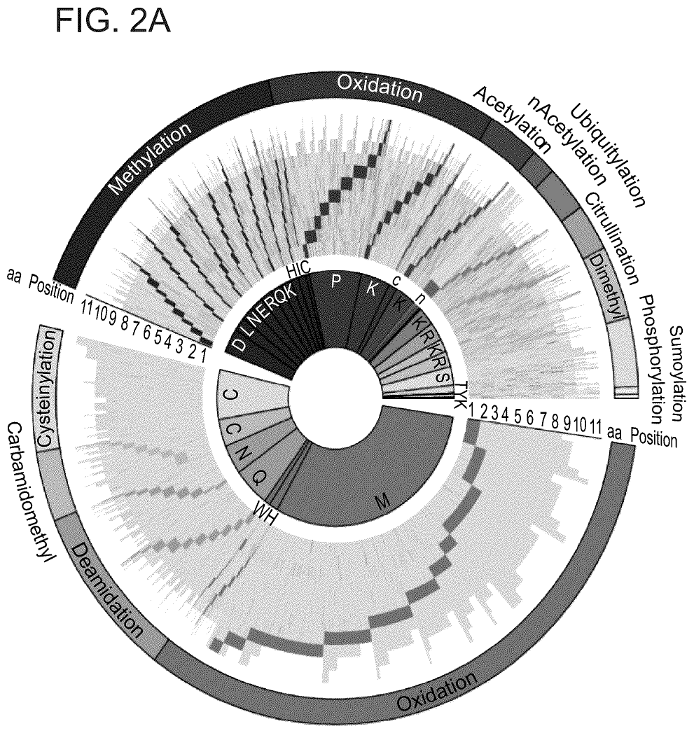

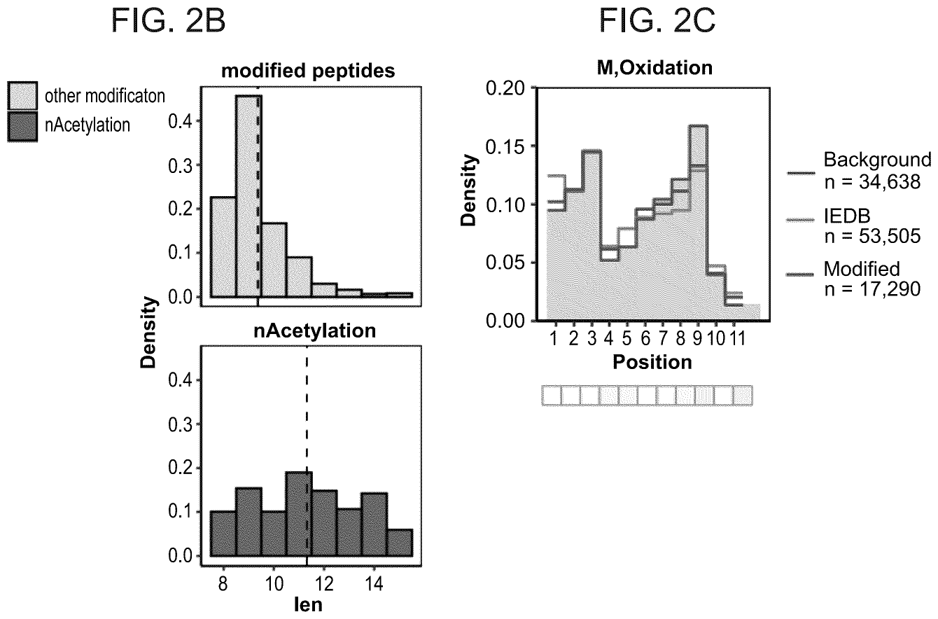

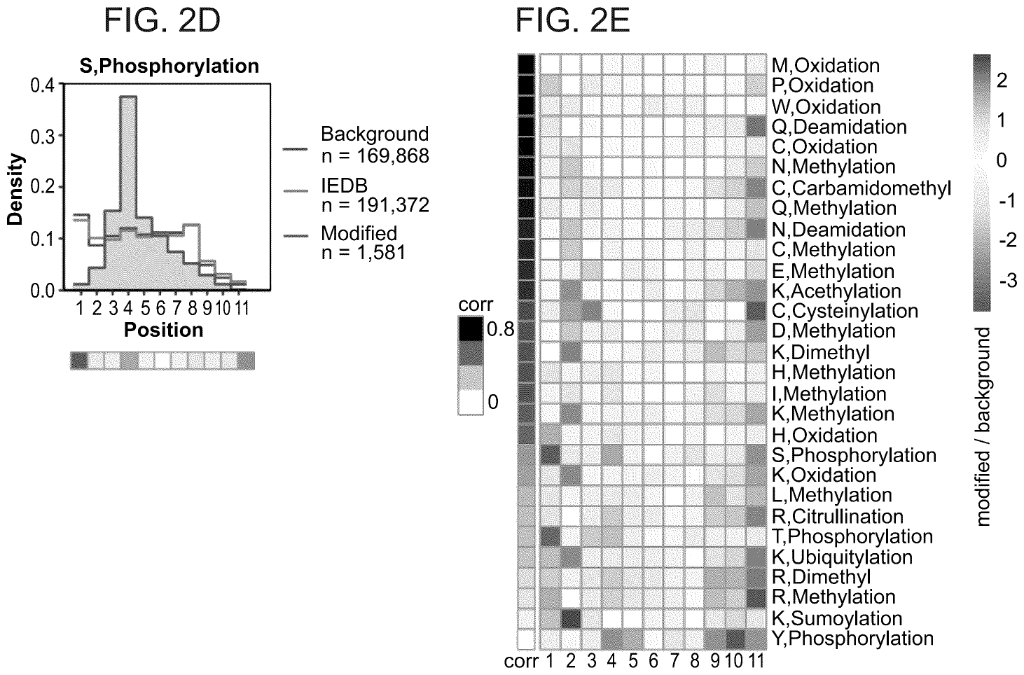

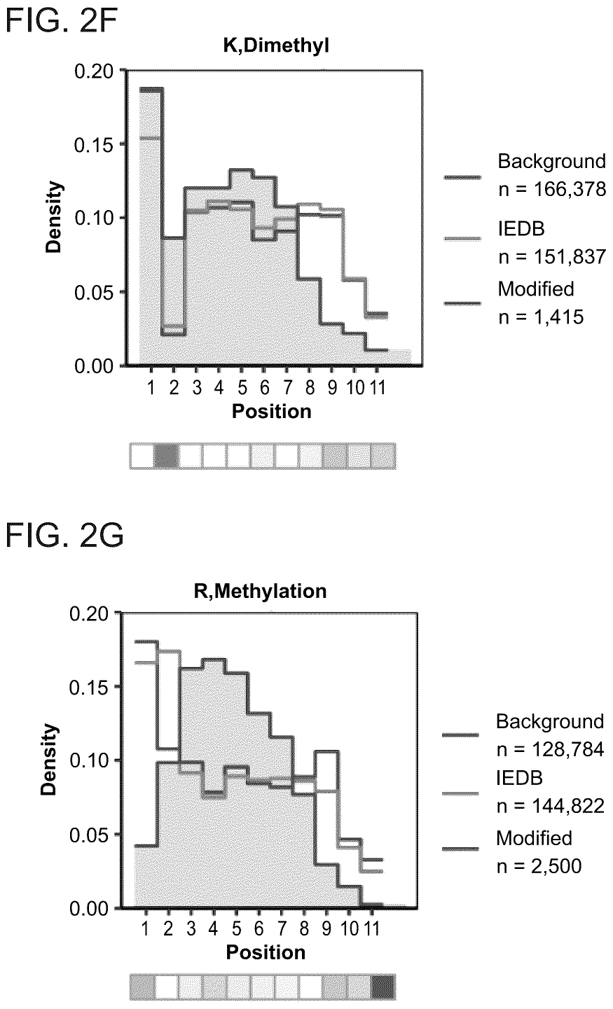

An unbiased search of 29 modifications in the immunopeptidome highlighted PTM-driven binding preferences—Peptide binding to major histocompatibility complex (MHC) molecules depends on the biochemical properties of both the peptide and MHC structure. The most critical residues for MHC binding are the ones that fit into the anchor pockets in the MHC groove, typically the second and carboxy-terminal positions⁴¹. By contrast. T-Cell receptors recognition motif is determined by the MHC-peptide complex and therefore most strongly influenced by the residues in position 3 to 7 of the HLA peptide^{42,43}. Given the generated global view of post-translationally modified peptides, whether a given PTM has the tendency to be in certain positions within the HLA peptide was explored. To capture the motifs of the full peptide repertoire, the criteria were loosened and a global FDR correction was used. A broad view across different types of modifications revealed that some modifications have a distinct site preference (FIG. 2A). For example, as previously shown^{10,11}, serine phosphorylation predominantly falls in the 4^{th} position of the HLA-bound peptide. Further, oxidation and cysteinylation are enriched at the end of the peptide (towards the c-terminus), cysteinylation is underrepresented at the second position, and carbamidomethyl is enriched in the third position. By contrast, other technical modifications, which are mainly due to processing, like deamidation, distribute evenly across the peptide. Furthermore, peptides with n-terminus acetylation, meaning they originate from the n-terminus of their parent protein, are longer on average from other peptide subsets (FIG. 2B).

Following, whether the distribution of these PTMs is distinct from the underlying distributions of the amino acid residues that they modify was explored. In addition, an unbiased and broader background distribution was also examined by collectively defining all of the reported epitopes in the IEDB⁴⁴ database. As expected, when examining a known technical modification, like methionine oxidation, the correlation between the oxidized methionine position distribution and the un-modified methionine distribution was very high (Pearson 0.96, p value=1.05e-6) (FIG. 2C). This suggests that the modification occurred randomly across the peptide during sample preparation or that it does not affect the binding motif at all (F-test; p value=0.543). Known motifs, such as the tendency of serine phosphorylation modification at position 4^{10,11}, were also emphasized as low correlation in this analysis (Pearson 0.41, p value=0.21) as there was a strong deviation between the phosphorylation and underlying serine distributions (FIG. 2D; F-test; p value=2.2e-16). This is despite any experimental or computational enrichments for specific modifications, as a broad search was used that was not modification-specific.

Given that the correlation between the distributions of the modified and unmodified sites is a good indicator of novel PIM-driven motifs, all of the PTMs detected were ordered based on the correlation of their distribution to the background (FIG. 2E). This metric was used to highlight PTM-driven motifs. For example, lysine residues at the second position of the peptide, in the HLA binding pocket, are under-represented. However, modified lysine residue distributions (e.g. acetylated and methylated lysine) do not produce the same pattern (FIG. 2F). This suggests that unmodified lysine residues in the anchoring position are unfavorable for HLA binding and that the modified state of a lysine residue may be preferred. In contrast, modified arginine such as di/methylated arginine and citrullination are over-represented in positions 3 to 7, and therefore may impact the T-cell receptor recognition⁴² (FIG. 2G), as was previously shown to for other types of modifications. Interestingly, while cysteine modifications on peptides in MS analyses are considered to be introduced by sample processing, in the current analysis of the HLA landscape they have a distinct distribution motif where cysteine carbamidomethyl is enriched in positions 3-4 and cysteinylation is enriched in positions 7-8 (FIG. 2E).

MHC binding properties are altered by the modification state of the presented peptide—The biochemical binding properties of specific HLA haplotypes are the strongest determinants of peptide motifs. To examine whether the PTM-driven motif detected is associated with specific haplotypes, mono-allelic HLA immunopeptidomics data from Abelin et al⁶ were re-analyzed. The same multi-modification search as described above (Table 2 hereinabove) was conducted on the spectra obtained. Indeed, unique motifs that were haplotype-dependent were identified, using the unmodified amino acid distribution as a background. To focus on the most prominent features, a ‘site score’ was defined such that enrichment in the anchor positions will result in a positive score while enrichment in the middle of the peptide will result in a negative score. In case the PTM is present in many positions in the peptide, the score will be close to zero the tendency of the modification cannot be classified to be in a specific area. The PTMs and haplotypes contained in the dataset were then clustered by their site score (FIG. 3A). This analysis revealed that the same PTM might affect peptide-MHC-TCR interactions differently for different haplotypes. Intriguingly, among the specific HLA haplotypes that were analyzed, several HLA associations with human diseases were found. For example, HLA A*0301 was linked to increased risk for multiple sclerosis 4 and HLA B*5101 was linked to Behcet disease⁴⁶. The current analysis identified both haplotypes to be highly enriched with PTMs in the region that is predicted to affect TCR recognition. HLA-A*201 was previously reported to show a protective effect in EBV-related Hodgkin lymphoma patients⁴⁷ and in the current analysis was enriched with modifications on the anchoring position of the peptide. While it remains to be examined whether certain PTMs play a role in disease-associated manifestations, it has been reported that low HLA binding of disease associated epitopes can be increased by PTM⁴⁸.

Based on analysis of the detected peptide modifications, the resulting interactions could be classified into three groups: The first group is comprised of chemical mimics, where the modified amino acid is biochemically similar to a different amino acid that was known to be part of the motif. For example, an enrichment of deamidated asparagine in position 3 of the haplotype A0101 motif was identified. Deamidated asparagine is chemically similar to aspartic acid which appears in the A0101 binding motif at position 3 (FIG. 3B). As no unmodified peptide carrying asparagine bound to this haplotype was detected, this result suggests that the modification occurred on the peptide before being bound to the MHC, possibly due to removal of a glycosylation⁴⁹; and the modified asparagine enables the binding of the peptide to the HLA.

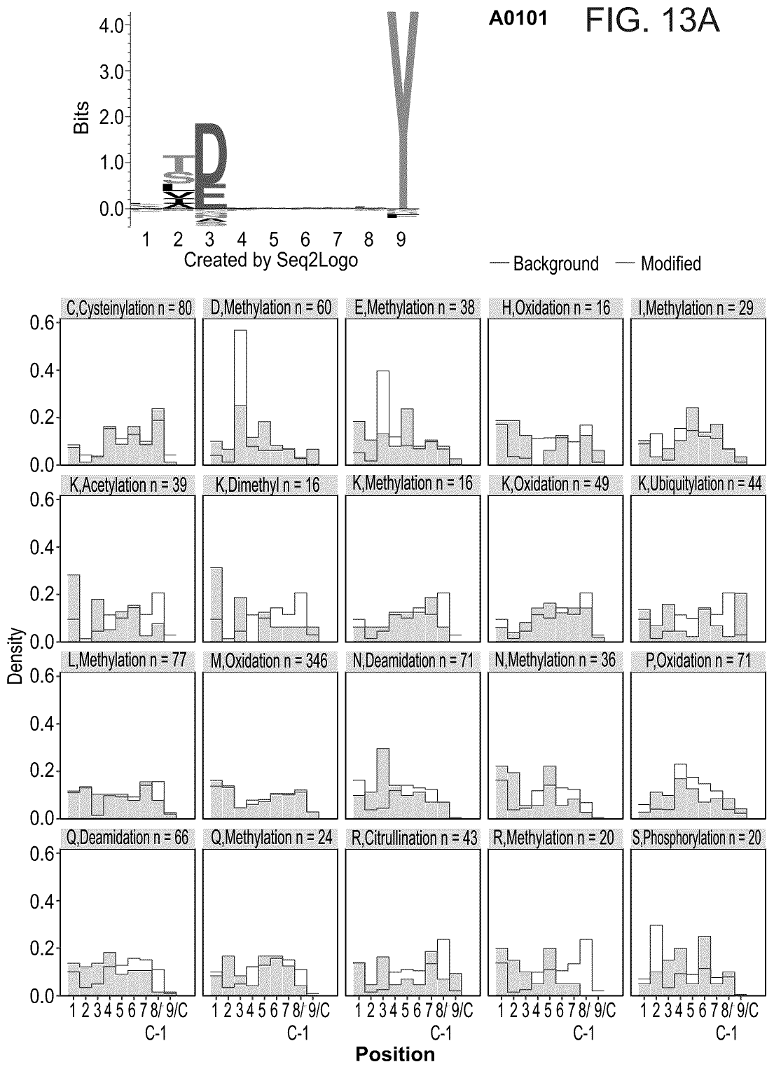

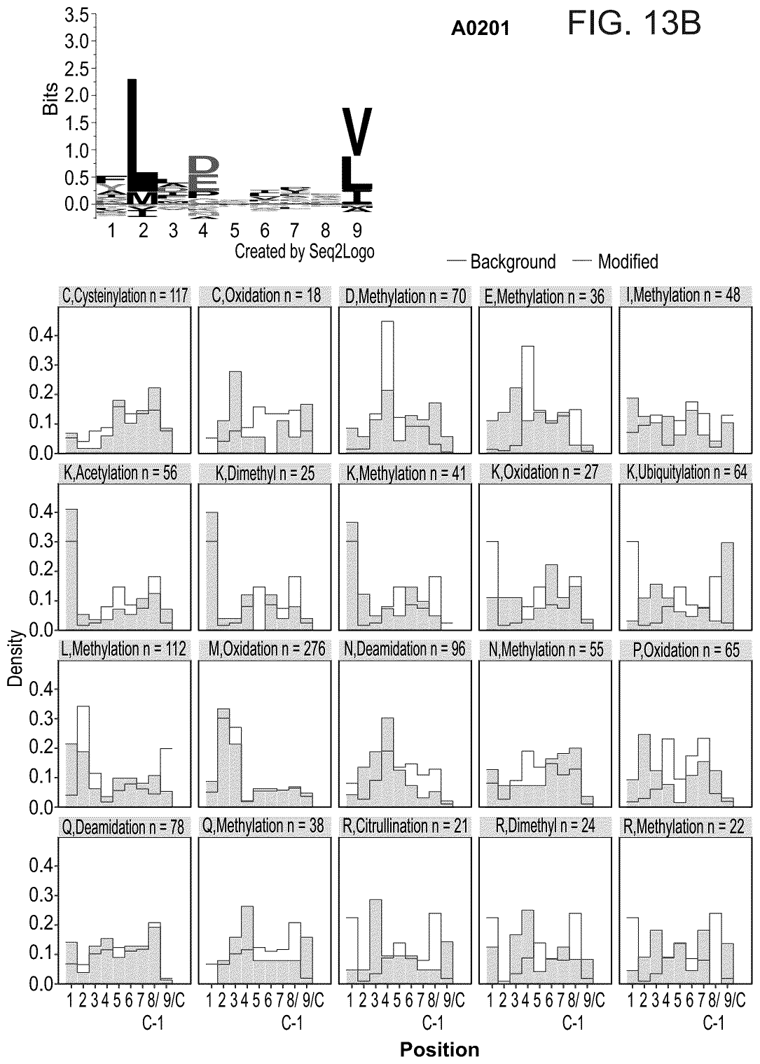

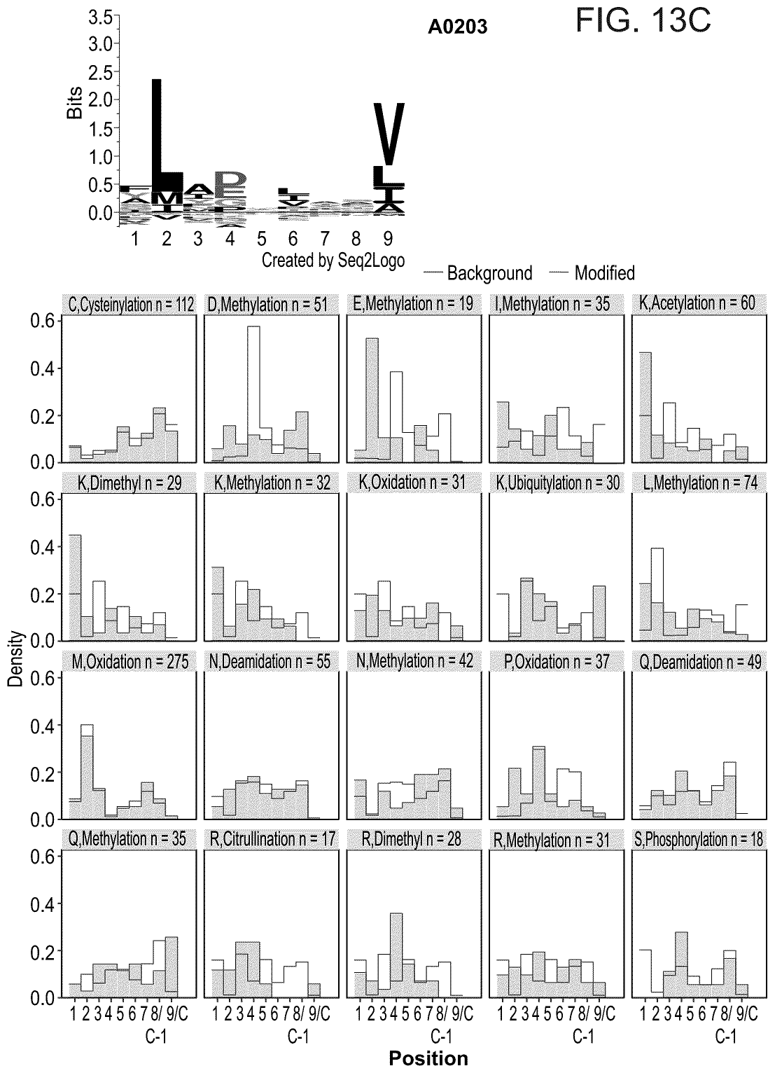

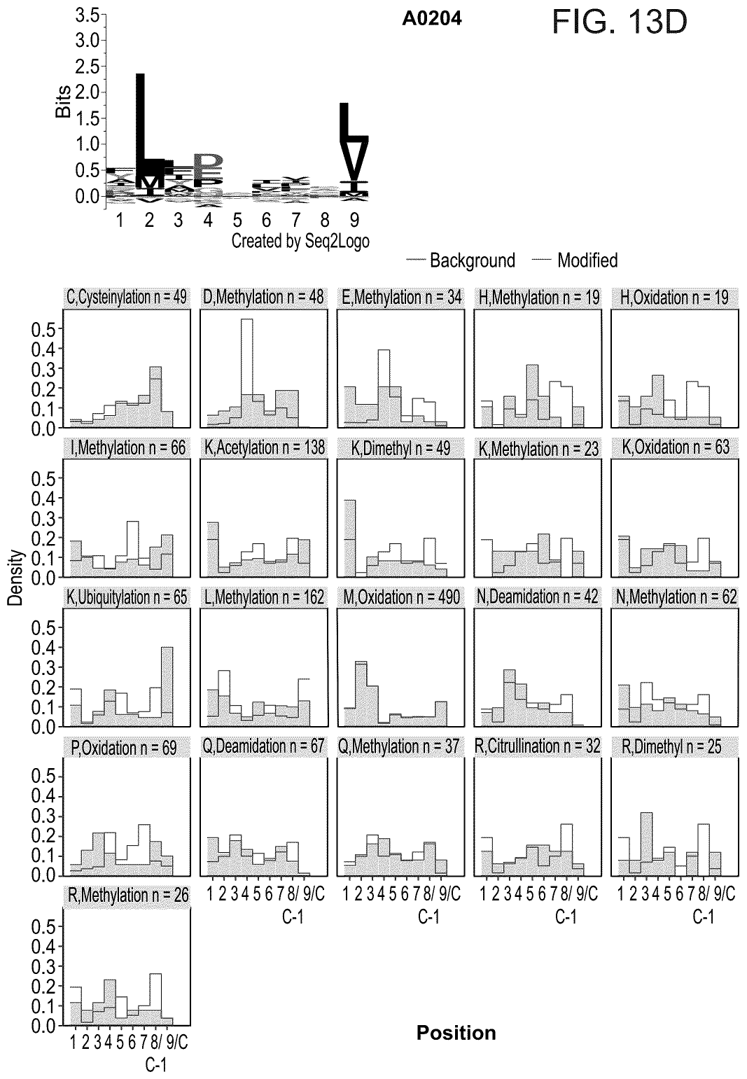

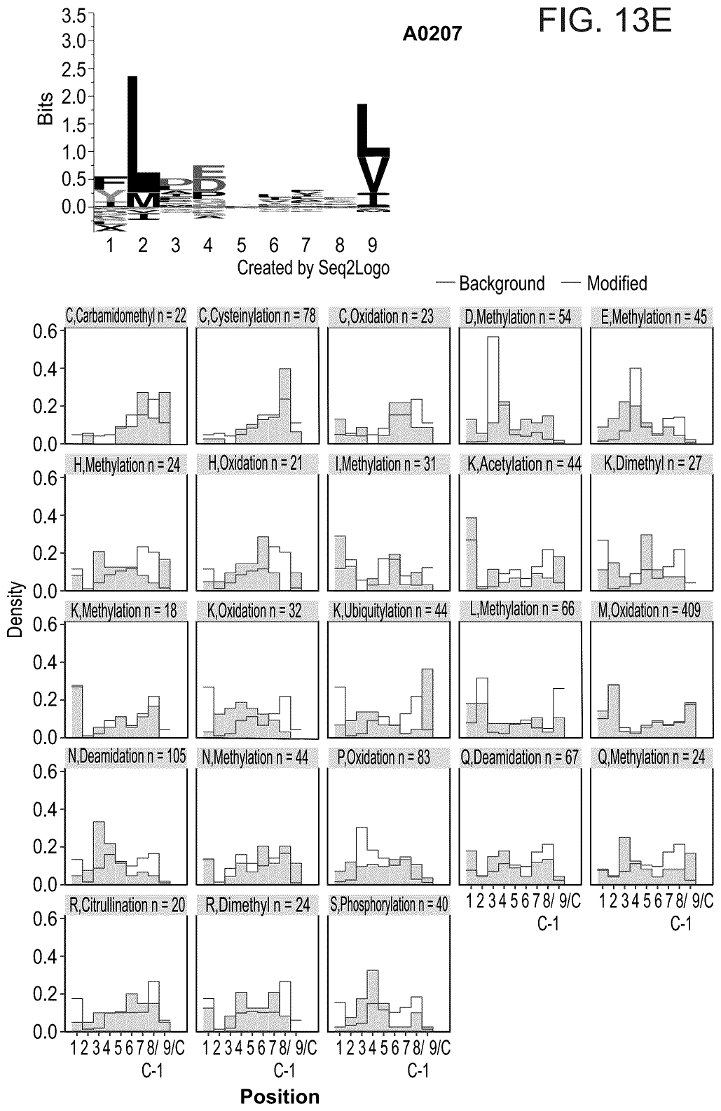

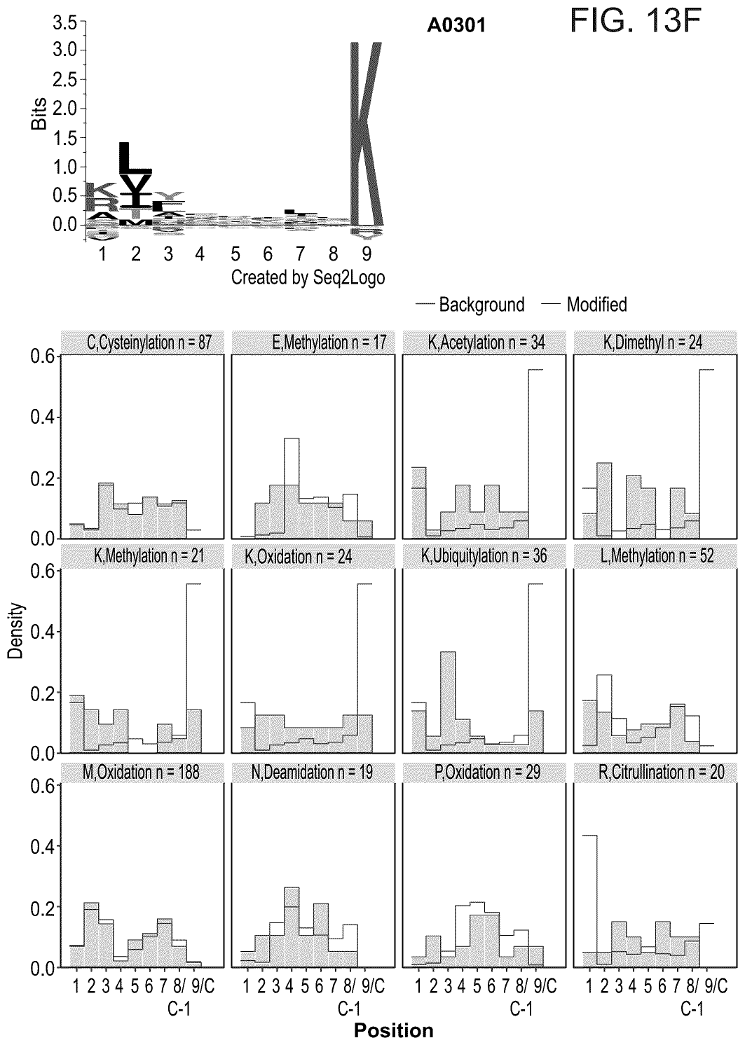

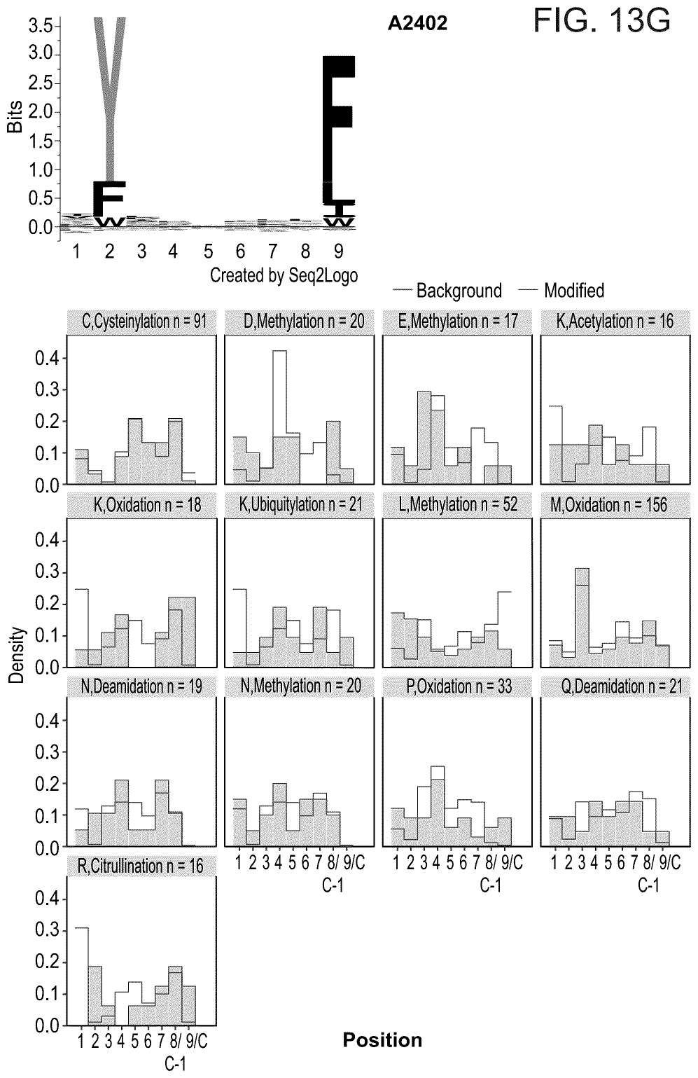

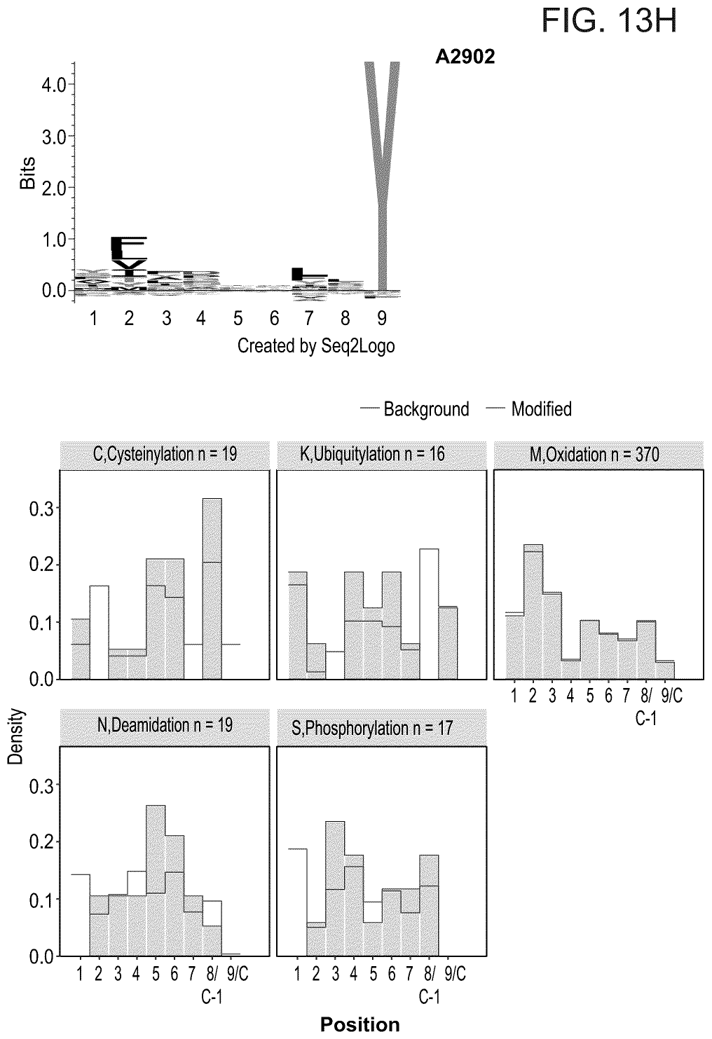

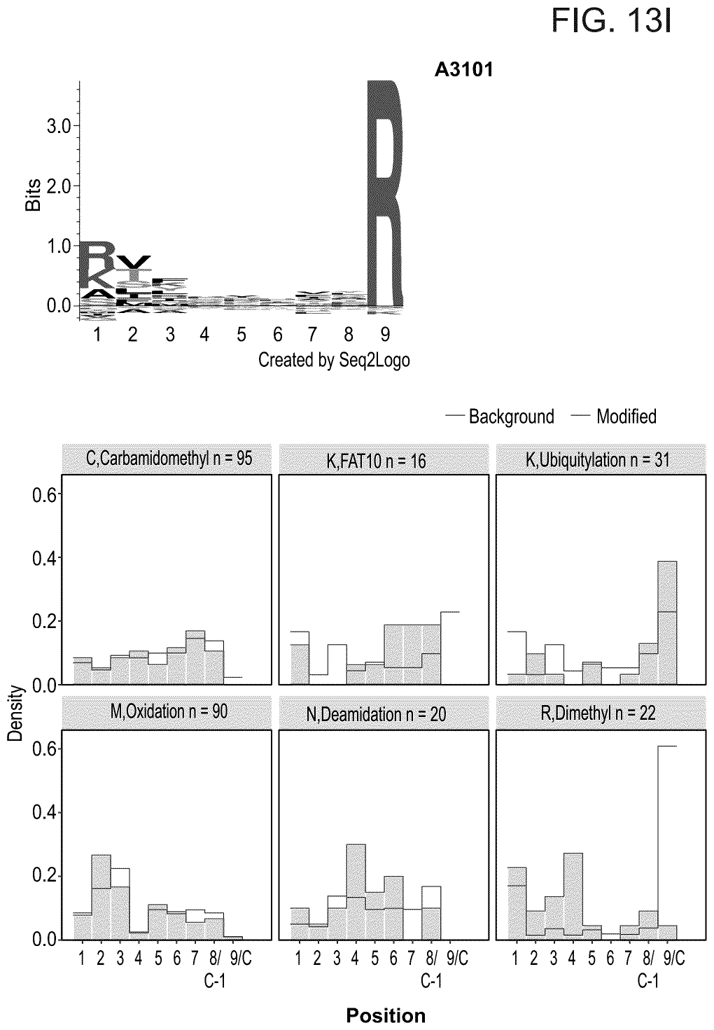

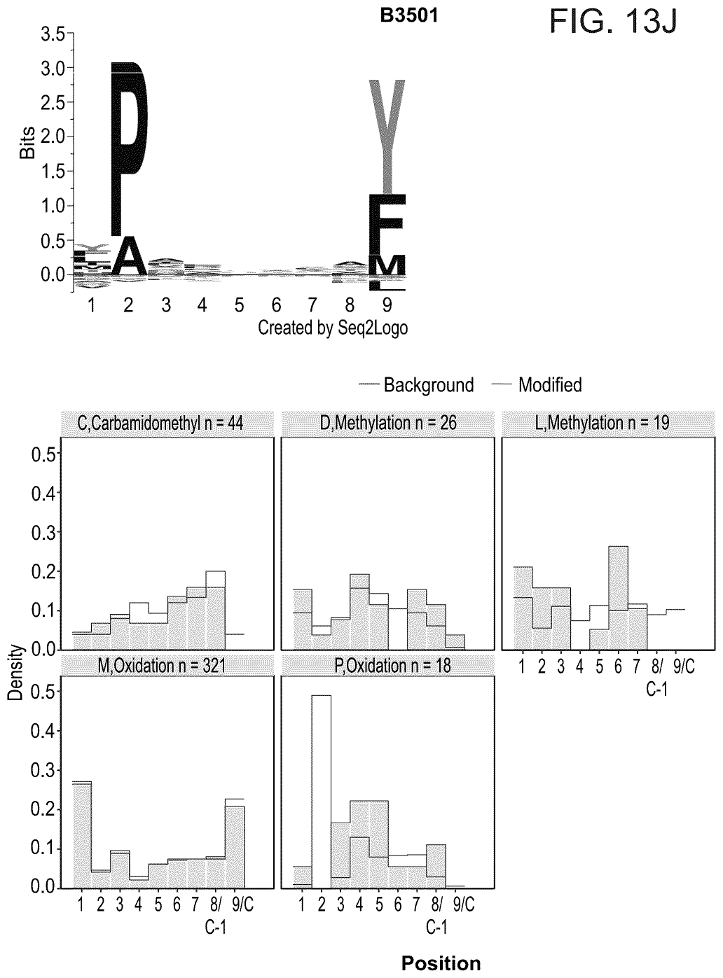

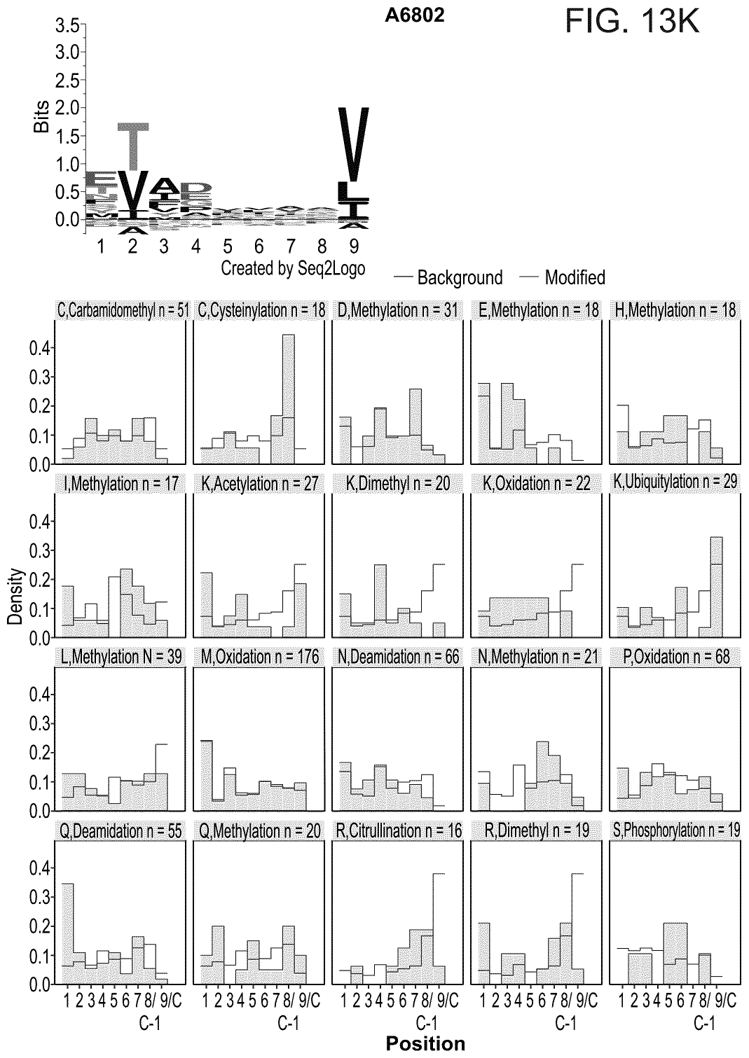

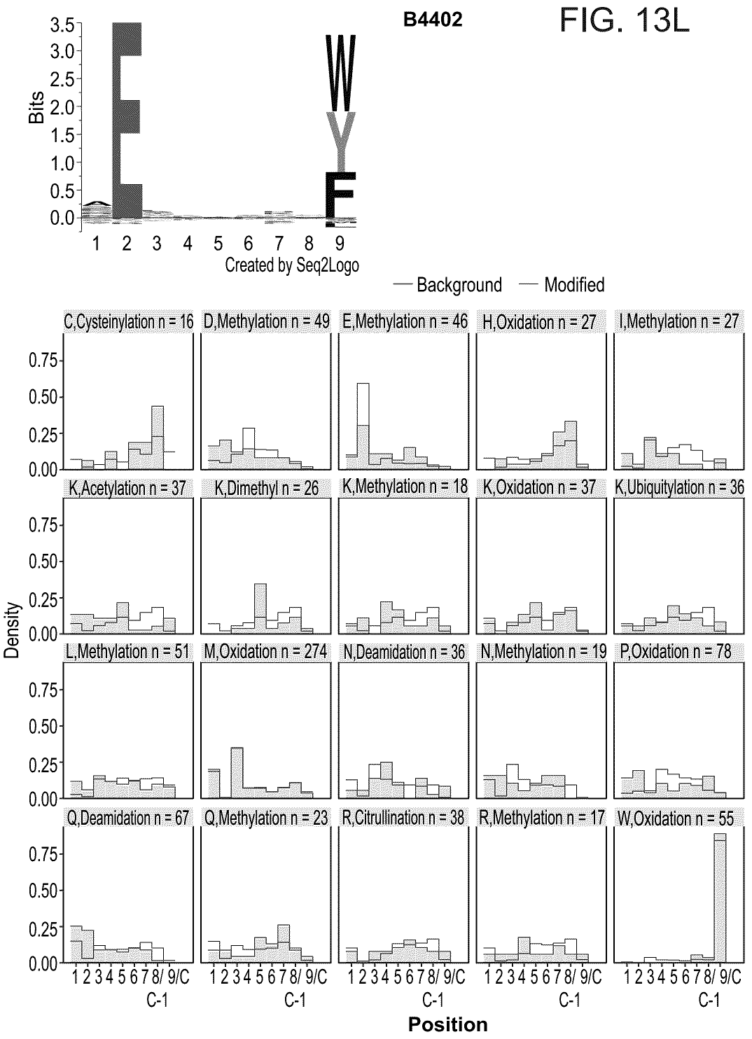

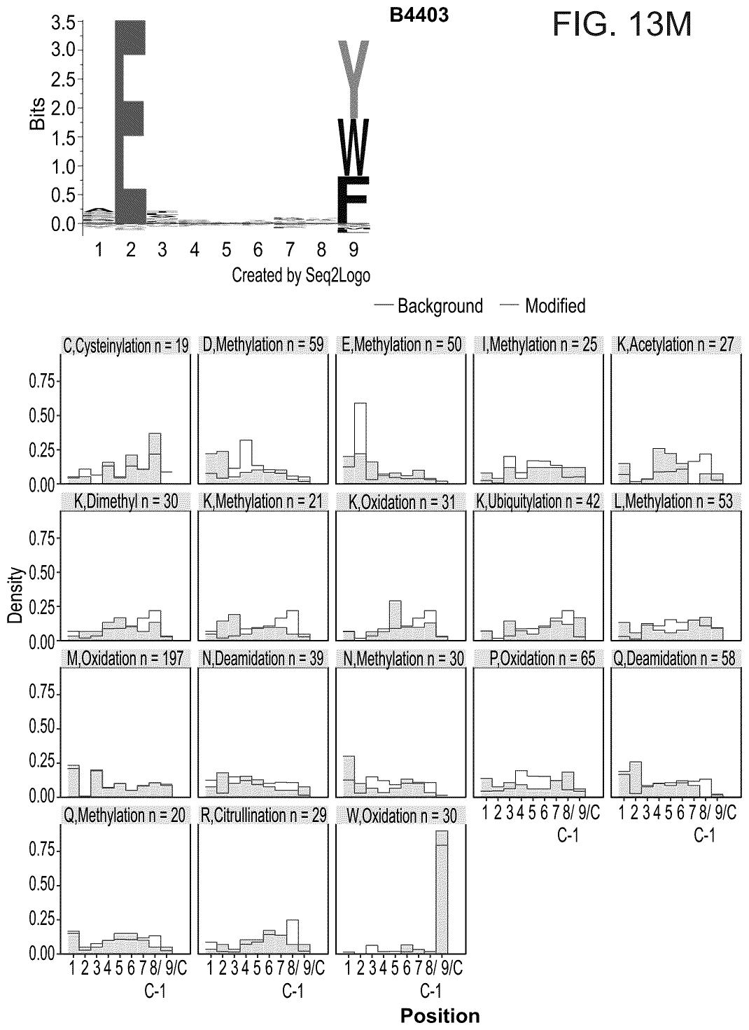

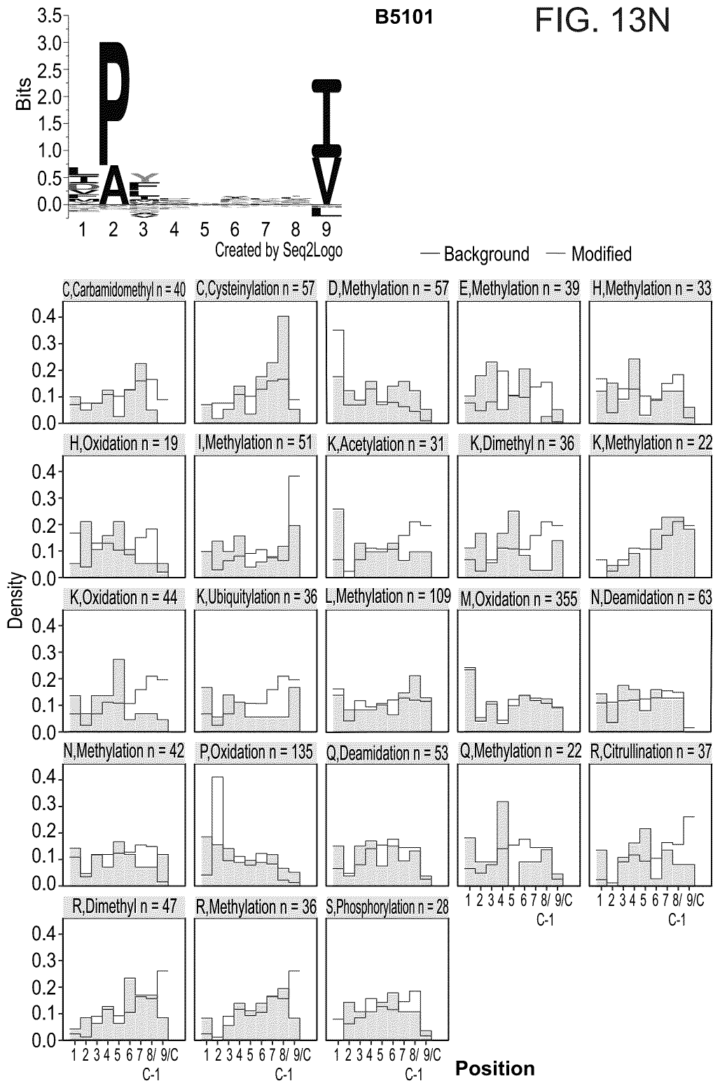

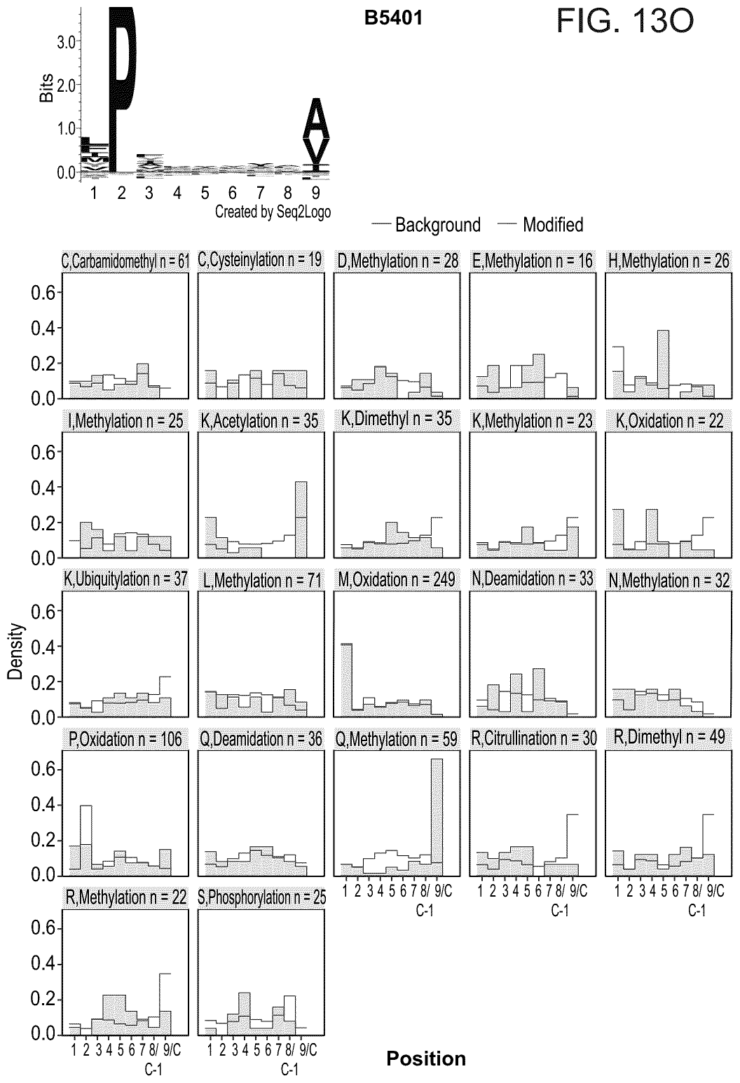

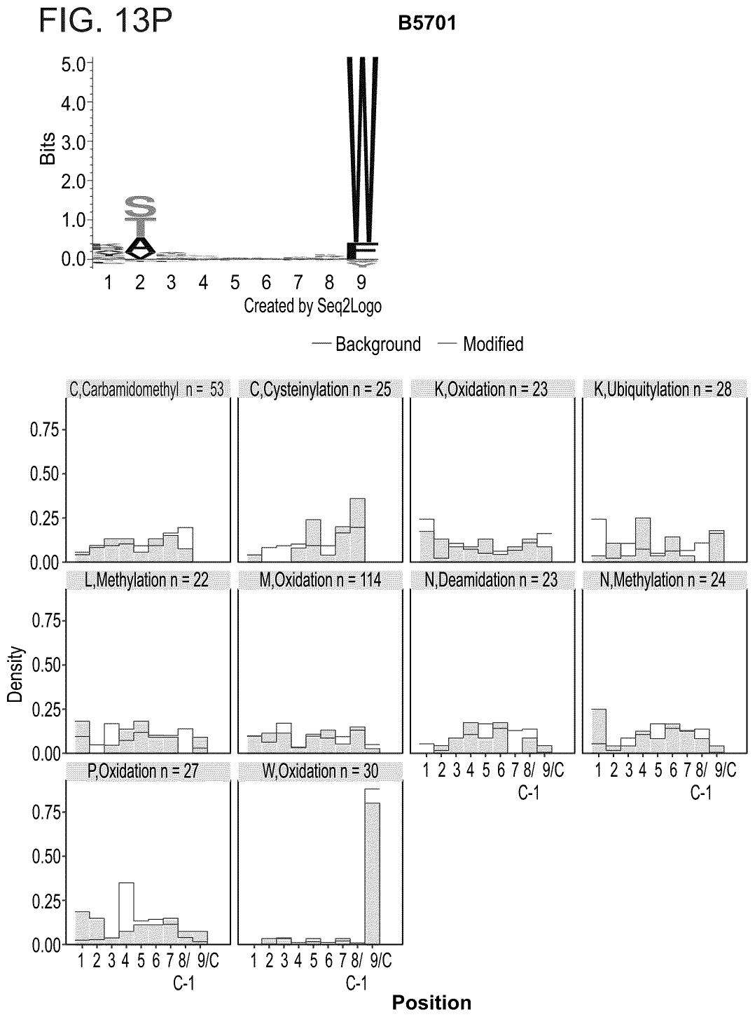

Enrichment of deamidated asparagine and glutamine at HLA haplotype A6802, B4402 and B4403 (FIGS. 13A-P) are additional examples of chemical mimics.

The second group contains PTMs that cause binding interference. This group is defined by PTMs that sterically hinder the interaction of the peptide with the MHC haplotype, creating an unfavorable binder. For example, acetylated lysine is under-represented in the C-terminus of haplotype A0301 (FIG. 3C) compared to the unmodified background. Importantly, this observation was applied for all of the modified lysines detected in this haplotype, suggesting that the modification of the carboxy-termini could be an immune evasion mechanism. Other examples for binding interference are methylated glutamic acid at anchor position 2 of haplotype B4402/3, and dimethylated arginine at the C-terminus position of haplotype A3101 (FIGS. 13A-P).

The third group are novel motifs where the modified amino acid creates a favorable binder peptide that is different from the known unmodified motif. It was shown that phosphoserine can replace glutamic acid at anchor position 2 of haplotype B4002¹³. In the generated dataset, methylated glutamine was detected at the peptide C-terminus in haplotype B5401 (FIG. 3D) and oxidized proline was observed at the anchor position two of haplotype A0201 (FIG. 3E). The latter observation is common to the whole haplotype superfamily A02 (FIGS. 13A-P).

Following, the possibility of a novel PTM binding motif was evaluated using structural modeling. To this end, two representative modified epitopes identified as binders of haplotype A0201 and one representative epitope identified as a binder to haplotype B5401 were chosen. All of them are shared across cancer cell lines and patient's tumor samples. Rosetta FlexPepDock⁵⁰ was used to model the structure of the interactions of these novels MHC-binding PTM motifs. K(ac)P(ox)SLEQSPAVL (SEQ ID NO: 10817 having the recited modifications), KP(ox)LKVIFV (SEQ ID NO: 10827 having the recited modification) and MPTLPPYQ(me) (SEQ ID NO: 10818 having the recited modification). For each such motif, both the modified and unmodified peptides were modeled and their calculated binding energies and structures (“Reweighted score”) were compared. In both cases, the interactions between the MHC and the modified peptide interactions were predicted to be considerably stronger, suggesting the complex is more stable than the non-modified counterpart (FIGS. 3F-G and 5) in agreement with the predictions from PROMISE immunopeptidomics analysis. In the case of peptide K(ac)P(ox)SLEQSPAVL (SEQ ID NO: 10817, having the recited modifications) binding to HLA-A*0201, the model suggests that the hydroxyl group of peptide P(ox)-2 forms a stabilizing hydrogen bond with receptor E-87 (FIG. 3F). Overall, our models recapitulate an interaction similar to a solved structure of HLA-A2 in which T-2 forms hydrogen bonds with receptor K-90 and E-87 (1TVB⁵¹). As for K(ac)-1, in some of the models it interacts with the aliphatic part of receptor K-90, while in others it further stabilizes the peptide. In the case of peptide MPTLPPYQ(me) (SEQ ID NO: 10818 having the recited modification) binding to HLA-5401, Q-8 is positioned in the highly hydrophobic pocket that binds the canonical aliphatic c-terminal peptide position. Methylation allows the otherwise polar (negative) side chain of glutamine to approach (“fill”) the pocket and thereby stabilize the complex (FIG. 3G).

### Example 3

### Identification of Modified HLA I-Bound Peptides Expressed on Cancer Cells

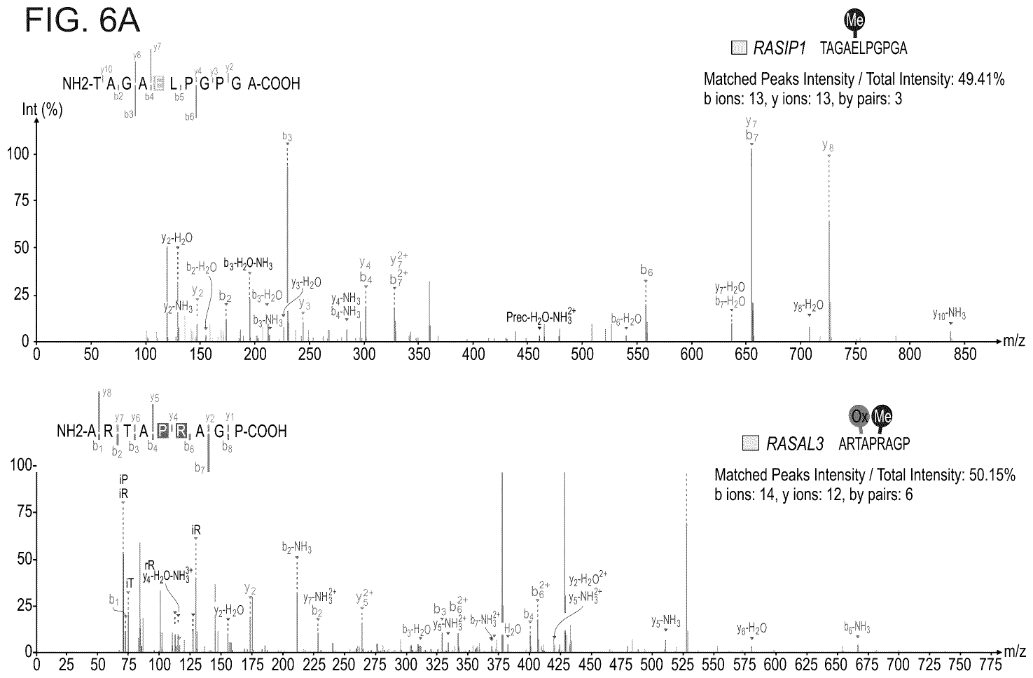

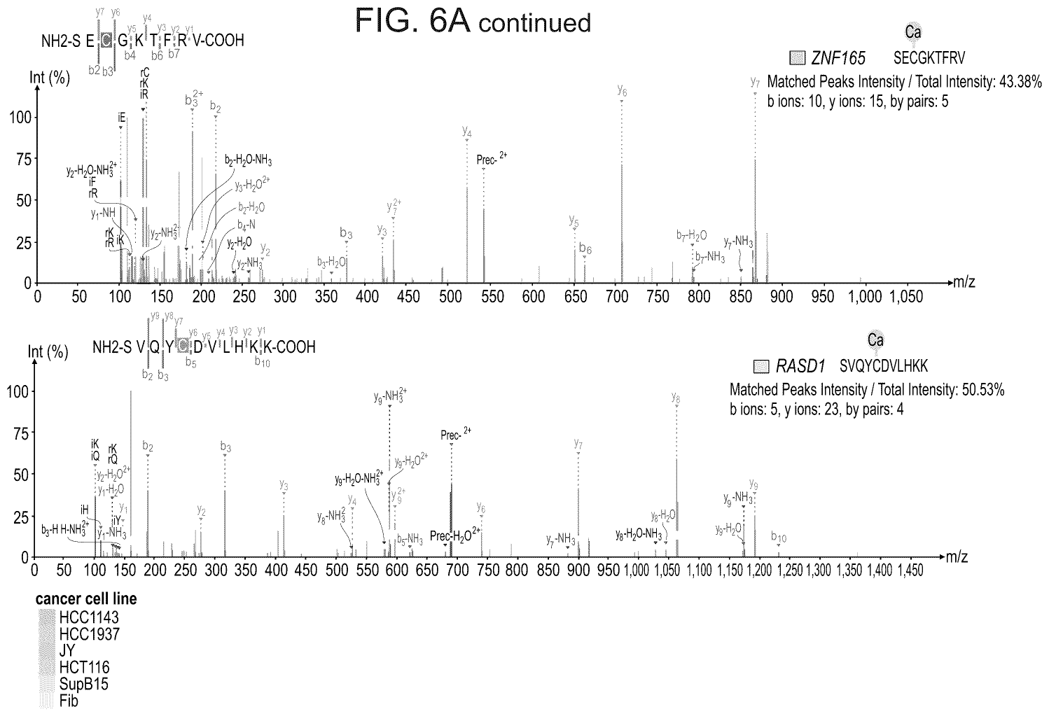

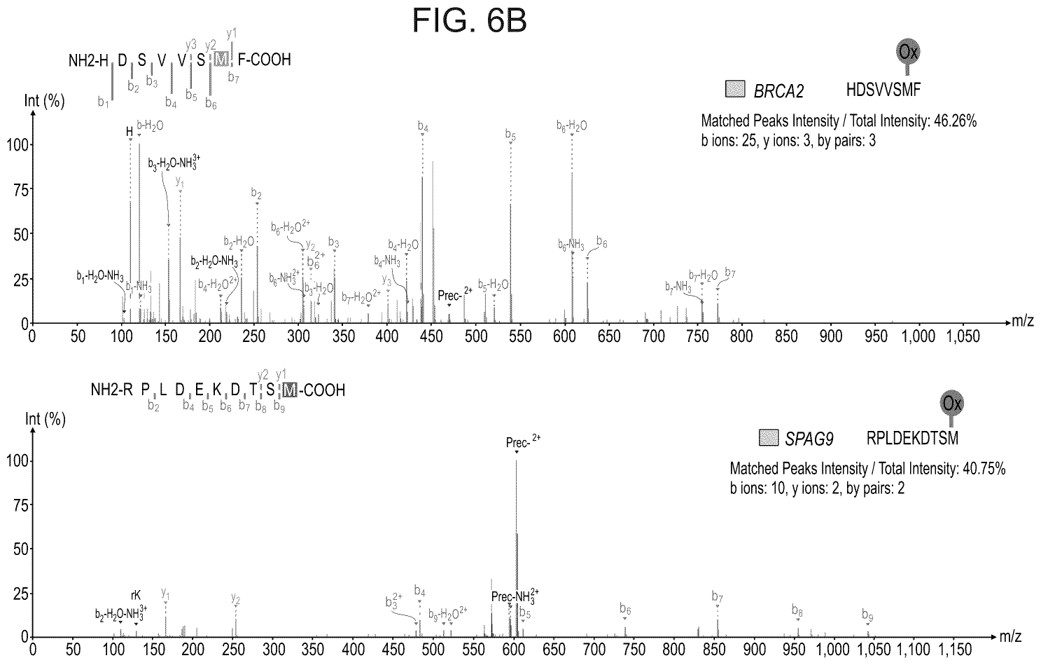

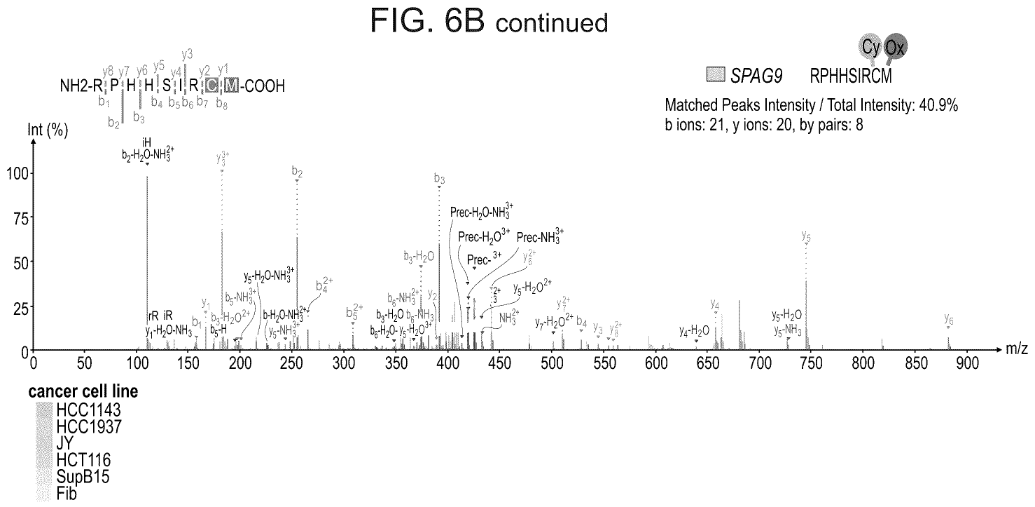

Among the identified modified peptides, cancer-specific signatures, across different cancer cell lines, were identified. Overall, the modified HLA-1 bound peptides detected on tumor cells are presented in Table 3 hereinabove. In addition, in numerous cases the presented modified peptides were unique to a specific cancer type (FIG. 4A, Table 3 hereinabove). It was hypothesized that this analysis may be influenced by the different protein composition in each cell line or the HLA haplotype and cancer-specific modification pathways. Furthermore, the dataset was searched for matching unmodified peptide, a peptide with the same amino acid sequence without the corresponding PTM (FIG. 4A—right panel). Next, the correlation score for the modified and unmodified peptide pairs was calculated (FIG. 4A; green scale bar). As expected, in a known technically produced modification, like oxidized methionine, 40% of the modified peptides had an unmodified match in the dataset and therefore a higher correlation score. At the top of the heatmap are modifications with a low correlation such as acetylation and citrullination which generally did not have unmodified counterparts. By contrast, some peptides with phosphorylation, dimethylation, and ubiquitination had a matching unmodified version, possibly highlighting their reversible nature and the fact that many proteins exist in the cell in both modified and unmodified states. While some modification types have higher correlation scores than others, peptides without unmodified counterparts in all PTM categories were revealed. For example, peptides from SPAG9 and ZNF165 with oxidations, cysteinylation, and carbamidomethylation were identified. Both proteins are examples of cancer-testis antigens that are not expressed in healthy adult tissues, and therefore may serve as putative targets for cancer immunotherapies (FIG. 4A). For all of these examples, the MS spectra ions had high confidence and matched the claimed peptide sequence including the identified PTM (FIGS. 6A-B).

To determine whether the signatures are also specific to the cancer state in clinical settings, immunopeptidomics data from a cohort of triple-negative breast cancer and adjacent tissue⁴⁰ were analyzed (Table 3 hereinabove). This analysis revealed that several modifications are significantly reduced in abundance in the tumor immunopeptidome, including carbamidomethyl and citrullination (FIG. 4B). Further, cysteinylated peptides are significantly increased in the tumor immunopeptidome. These changes may reflect alterations in metabolic pathways or peptide processing. For example, it is known that triple-negative breast cancer is addicted to cysteine^{52,53}, potentially explaining the increase in cysteinylated immunopeptides.

Given the growing interest in identifying antigenic targets for immunotherapy, whether the identified modified peptides originated from cancer-associated or testis antigens was examined. 244 peptides that originated from a protein annotated as a testis antigen (from CT Antigens Database⁵⁴) and 400 peptides that were highly shared across cancer cohorts (FIG. 4C) were identified, indicating the identified modified peptides presented in Table 3 hereinabove may be good targets for therapies. Many of these proteins are also annotated as oncogenes, cancer drivers or tumor suppressors⁵⁵, suggesting that the modifications may modulate the disease pathogenesis.

To validate that the modified peptides identified with PROMISE are able to bind to HLA, the subset of modified peptides that were identified in immunopeptidomics of an HLA-A0201 cell line and that were not identified in IEDB in their unmodified form were filtered (FIG. 4D). Further, whether the difference in the detection of the modified peptides and their unmodified counterparts was due to their relative ability to bind HLA-A0201 was examined. Structural modeling demonstrated that the methylation on the lysine in position 6 of TLIESKLPV (SEQ ID NO: 10823) is located between 3 other positively charged residues (H-98, R121, and H-138; FIG. 4E). Methylation of K-6 removes its positive charge and thereby alleviates electrostatic repulsion. In addition, the methyl group is nicely packed into the hydrophobic MHC groove. This then causes a more stable peptide-MHC interaction as reflected in a lower reweighted score. To assess the role of peptide modification in altering MHC binding 6 modified peptides and their unmodified counterparts were synthesized and their binding was examined using a binding assay (ProImmune). In these setting 4 of the synthesized modified peptides were confirmed as HLA binders. Of these, three were shown to bind more strongly than their unmodified counterparts (FIG. 4F). Specifically, TLIESK(me)LPV (SEQ ID NO: 10823 having the recited modification) was shown to bind more strongly in its modified form as predicted by the structural model. Of note, the fact that 2 of the synthesized modified peptides did not bind HLA in these experimental settings may be due to absence of all chaperones supporting loading of the peptides to the MHC molecule in this in-vitro settings.

Of note, the data have also suggested that remnants of ubiquitin tails on peptides, after proteasome degradation, may be detected on peptides bound to MHC molecules. Recently it was found that a proximal ubiquitin modification may undergo degradation with its substrate^{57,59}. As a consequence, a couple of residues from the ubiquitin tail remain attached to the proteasome-cleaved peptide. Here the present inventors report, for the first time, that remnants from ubiquitin and ubiquitin-like (UBL) modifiers remain on the peptide substrate following proteasome cleavage and can be identified in immunopeptidomics (Table 2 hereinabove and FIG. 14).

### Example 4

### Identification of Novel HLA I-Bound Peptides Using the Novel Protein Modification Integrated Search Engine

Using the above described methodology, the present inventors have identified several novel modified peptides in which the modification is suspected to be technical and hypothesized that they are presented on cancerous cells in an un-modified state (Table 4 hereinabove).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Obara, W. et al. Present status and future perspective of peptide-based vaccine therapy for urological cancer. Cancer Sci. 109, 550-559 (2018).
2. Jiang. D., Niwa. M., Koong. A. C. & Diego. S. Cancer immunotherapy: moving forward with peptide T cell vaccines. Eur. J. Vasc. Endovasc. Surg. 49, 48-56 (2016).
3. Xia. A.-L., Wang, X.-C., Lu, Y.-J., Lu, X.-J. & Sun, B. oncotarget Chimeric-antigen receptor T (CAR-T) cell therapy for solid tumors: challenges and opportunities. Oncotarget 8, 90521-90531 (2017).
4. Finn. O. J. & Rammensee. H. G. Is it possible to develop cancer vaccines to neoantigens, what are the major challenges, and how can these be overcome?: Neoantigens: Nothing new in spite of the name. Cold Spring Harb. Perspect. Biol. 10. (2018).
5. Jurtz, V. et al. NetMHCpan-4.0: Improved Peptide-MHC Class I Interaction Predictions Integrating Eluted Ligand and Peptide Binding Affinity Data. J. Immunol. 199, 3360-3368 (2017).
6. Abelin, J. G. et al. Mass Spectrometry Profiling of HLA-Associated Peptidomes in Mono-allelic Cells Enables More Accurate Epitope Prediction. Immunity 46, 315-326 (2017).
7. O'Donnell, T. J. et al. MHCflurry: Open-Source Class I MHC Binding Affinity Prediction. Cell Syst. 7, 129-132.e4 (2018).
8. Gfeller. D. et al. The Length Distribution and Multiple Specificity of Naturally Presented HLA-I Ligands. J. Inmunol. 201, 3705-3716 (2018).
9. Bulik-Sullivan, B. et al. Deep learning using tumor HLA peptide mass spectrometry datasets improves neoantigen identification. Nat. Biotechnol. 37, 55-71 (2019).
10. Alpizar. A. et al. A molecular basis for the presentation of phosphorylated peptides by HLA-B antigens. Mol. Cell. Proteomics 16, 181-193 (2017).
11. Bassani-Sternberg, M. et al. Direct identification of clinically relevant neoepitopes presented on native human melanoma tissue by mass spectrometry. Nat. Commun. 7, 13404 (2016).
12. Mohammed. F. et al. The antigenic identity of human class I MHC phosphopeptides is critically dependent upon phosphorylation status. Oncotarget 8, 54160-54172 (2017).
13. Marcilla, M. et al. Increased diversity of the hla-b40 ligandome by the presentation of peptides phosphorylated at their main anchor residue. Mol. Cell. Proteomics 13, 462-474 (2014).
14. Marino. F. et al. Arginine (Di)methylated Human Leukocyte Antigen Class I Peptides Are Favorably Presented by HLA-B*07. J. Proteome Res. 16, 34-44 (2017).
15. Malaker, S. A. et al. Identification of glycopeptides as posttranslationally modified neoantigens in Leukemia. Cancer Inmunol. Res. 5, 376-384 (2017).
16. Petersen, J., Purcell, A. W. & Rossjohn, J. Post-translationally modified T cell epitopes: Immune recognition and immunotherapy. Journal of Molecular Medicine vol. 87 1045-1051 (2009).
17. Mommen. G. P. M. et al. Expanding the detectable HLA peptide repertoire using electron-transfer/higher-energy collision dissociation (EThcD). Proc. Natl. Acad. Sci. U.S.A. 111, 4507-4512 (2014).
18. Bassani-Stemberg. M., Pletscher-Frankild. S., Jensen. L. J. & Mann. M. Mass spectrometry of human leukocyte antigen class I peptidomes reveals strong effects of protein abundance and turnover on antigen presentation. Mol Cell Proteomics 14, 658-673 (2015).
19. Chong, C. et al. High-throughput and Sensitive Immunopeptidomics Platform Reveals Profound Interferonγ-Mediated Remodeling of the Human Leukocyte Antigen (HLA) Ligandome. Mol. Cell. Proteomics 17, 533-548 (2018).
20. Ott, P. A. et al. An immunogenic personal neoantigen vaccine for patients with melanoma. Nature 547, 217-221 (2017).
21. Sahin. U. & Türeci, Ö. Personalized vaccines for cancer immunotherapy. Science (80-.). 359, 1355-1360 (2018).
22. Keskin, D. B. et al. Neoantigen vaccine generates intratumoral T cell responses in phase Ib glioblastoma trial. Nature 565, 234-239 (2019).
23. Chu. Y., Liu, Q., Wei, J. & Liu, B. Personalized cancer neoantigen vaccines come of age. Theranostics 8, 4238-4246 (2018).
24. Schumacher, T. N., Scheper. W. & Kvistborg, P. Cancer Neoantigens. Annu. Rev. Immunol. 37, 173-200 (2019).
25. Vizcaino, J. A. et al. The human immunopeptidome project: A roadmap to predict and treat immune diseases. Molecular and Cellular Proteomics vol. 19 31-49 (2020).
26. Sulzer, D. et al. T cells from patients with Parkinson's disease recognize α-synuclein peptides. Nature 546, 656-661 (2017).
27. Karasaki. T. et al. Prediction and prioritization of neoantigens: integration of RNA sequencing data with whole-exome sequencing. Cancer Sci. 108, 170-177 (2017).
28. Hoof. I. et al. NetMHCpan, a method for MHC class i binding prediction beyond humans. Immunogenetics 61, 1-13 (2009).
29. Peters, B. & Sette, A. Generating quantitative models describing the sequence specificity of biological processes with the stabilized matrix method. BMC Bioinformatics 6, 1-9 (2005).
30. Lundegaard, C. et al. NetMHC-3.0: accurate web accessible predictions of human, mouse and monkey MHC class I affinities for peptides of length 8-11. Nucleic Acids Res. 36, 509-512 (2008).
31. Pinkse. M. W. H., Uitto, P. M., Hilhorst, M. J., Ooms, B. & Heck, A. J. R. Selective isolation at the femtomole level of phosphopeptides from proteolytic digests using 2D-NanoLC-ESI-MS/MS and titanium oxide precolumns. Anal. Chenm. 76, 3935-3943 (2004).
32. Zhou, H. et al. Enhancing the Identification of Phosphopeptides from Putative Basophilic Kinase Substrates Using Ti (IV) Based IMAC Enrichment. Mol. Cell. Proteomics 10. M110.006452 (2011).
33. Rush, J. et al. Immunoaffinity profiling of tyrosine phosphorylation in cancer cells. Nat. Biotechnol. 23, 94-101 (2005).
34. Wagner, S. A. et al. A proteome-wide, quantitative survey of in vivo ubiquitylation sites reveals widespread regulatory roles. Mol. Cell. Proteomics 10, M111.013284 (2011).
35. Solleder. M. et al. Mass spectrometry based immunopeptidomics leads to robust predictions of phosphorylated HLA class I ligands. Mol. Cell. Proteomics mcp.TIR119.001641 (2019) doi:10.1074/mcp.TIR119.001641.
36. Na, S. & Pack. E. Software eyes for protein post-translational modifications. Mass Spectrom. Rev. 34, 133-147 (2015).
37. Kong. A. T., Leprevost. F. V. Avtonomov, D. M., Mellacheruvu. D. & Nesvizhskii. A. I. MSFragger: ultrafast and comprehensive peptide identification in mass spectrometry-based proteomics. Nat. Methods 14, 513-520 (2017).
38. Cox, J., Michalski, A. & Mann, M. Software Lock Mass by Two-Dimensional Minimization of Peptide Mass Errors. J. Am. Soc. Mass Spectrom. 22, 1373-1380 (2011).
39. Shraibman, B., Kadosh, D. M., Barnea, E. & Admon. A. Human Leukocyte Antigen (HLA) Peptides Derived from Tumor Antigens Induced by Inhibition of DNA Methylation for Development of Drug-facilitated Immunotherapy. Mol. Cell. Proteomics 15, 3058-3070 (2016).
40. Ternette, N. et al. Immunopeptidomic Profiling of HLA-A2-Positive Triple Negative Breast Cancer Identifies Potential Immunotherapy Target Antigens. Proteomics 18, 1700465 (2018).
41. Deres, K., Beck, W., Faath. S., Jung. G. & Rammensee, H. G. MHC/peptide binding studies indicate hierarchy of anchor residues. Cell. Immunol. 151, 158-167 (1993).
42. MacLachlan, B. J. et al. Using X-ray Crystallography. Biophysics, and Functional Assays to Determine the Mechanisms Governing T-cell Receptor Recognition of Cancer Antigens. J. Vis. Exp 120, 54991 (2017).
43. Wang, Y. et al. How an alloreactive T-cell receptor achieves peptide and MHC specificity, doi:10.1073/pnas.1700459114.
44. Vita, R. et al. The Immune Epitope Database (IEDB): 2018 update. Nucleic Acids Res. 47. D339-D343 (2019).
45. Fogdell-Hahn. A., Ligers, A., Gronning. M., Hillert, J. & Olerup. O. Multiple sclerosis: a modifying influence of HLA class I genes in an HLA class II associated autoimmune disease. Tissue Antigens 55, 140-148 (2000).
46. Wallace, G. R. HLA-B*51 the primary risk in Behçet disease. Proceedings of the National Academy of Sciences of the United States of America vol. 11 8706-8707 (2014).
47. Hjalgrim, H. et al. HLA-A alleles and infectious mononucleosis suggest a critical role for cytotoxic T-cell response in EBV-related Hodgkin lymphoma. Proc. Natl. Acad. Sci. U.S.A 107.6400-6405 (2010).
48. Sidney, J. et al. Low HLA binding of diabetes-associated CD8+ T-cell epitopes is increased by post translational modifications. BMC Immunol. 19, 12 (2018).
49. Skipper. J. C. A. et al. An HLA-A2-restricted tyrosinase antigen on melanoma cells results from posttranslational modification and suggests a novel pathway for processing of membrane proteins. J. Exp. Med. 183, 527-534 (1996).
50. Raveh, B., London, N. & Schueler-Furman. O. Sub-angstrom modeling of complexes between flexible peptides and globular proteins. Proteins Struct. Funct. Bioinforna. 78, 2029-2040 (2010).
51. Borbulevych, O. Y., Baxter, T. K., Yu. Z., Restifo, N. P. & Baker, B. M. Increased Immunogenicity of an Anchor-Modified Tumor-Associated Antigen Is Due to the Enhanced Stability of the Peptide/MHC Complex: Implications for Vaccine Design. J. Immunol. 174, 4812-4820 (2005).
52. Timmerman. L. A. et al. Glutamine Sensitivity Analysis Identifies the xCT Antiporter as a Common Triple-Negative Breast Tumor Therapeutic Target. Cancer Cell 24, 450-465 (2013).
53. Tang, X. et al. Cystine addiction of triple-negative breast cancer associated with EMT augmented death signaling. Oncogene 36.4235-4242 (2017).
54. Almeida, L. G. et al. CTdatabase: A knowledge-base of high-throughput and curated data on cancer-testis antigens. Nucleic Acids Res. 37, D816 (2009).
55. Lever. J., Zhao. E. Y., Grewal. J., Jones, M. R. & Jones, S. J. M. CancerMine: a literature-mined resource for drivers, oncogenes and tumor suppressors in cancer. Nat. Methods 16, 505-507 (2019).
56. Schuster. H. et al. Data Descriptor: A tissue-based draft map of the murine MHC class I immunopeptidome. Sci. Data 5, 1-11 (2018).
57. Sun. H. et al. Diverse fate of ubiquitin chain moieties: the proximal is degraded with the target, and the distal protects the proximal from removal and recycles. Proc. Natl. Acad. Sci. U.S.A 116, 7805-7812 (2019).
58. Ljunggren. H. G. et al. Empty MHC class I molecules come out in the cold. Nature 346, 476-480(1990).
59. Singh. S. K. et al. Synthetic Uncleavable Ubiquitinated Proteins Dissect Proteasome Deubiquitination and Degradation, and Highlight Distinctive Fate of Tetraubiquitin. J. Am. Chem. Soc. 138, 16004-16015 (2016).
60. Wolf-Levy, H. et al. Revealing the cellular degradome by mass spectrometry analysis of proteasome-cleaved peptides. Nat. Biotechnol. 36, 1110-1116 (2018).
61. Thomsen, M. C. F. & Nielsen, M. Seq2Logo: a method for construction and visualization of amino acid binding motifs and sequence profiles including sequence weighting, pseudo counts and two-sided representation of amino acid enrichment and depletion. Nucleic Acids Res. 40, W281-W287 (2012).
62. Vacic. V., Iakoucheva. L. M. & Radivojac. P. Two Sample Logo: A graphical representation of the differences between two sets of sequence alignments. Bioinformatics 22, 1536-1537 (2006).
63. Alam, N. & Schueler-Furman, O. Modeling peptide-protein structure and binding using monte carlo sampling approaches: Rosetta flexpepdock and flexpepbind, in Methods in Molecular Biology vol. 1561 139-169 (Humana Press Inc., 2017).
64. London. N., Lamphear, C. L., Hougland, J. L., Fierke, C. A. & Schueler-Furman, O. Identification of a novel class of famesylation targets by structure-based modeling of binding specificity. PLoS Comput. Biol. 7, (2011).
65. McMurtrey, C. et al. Toxoplasma gondii peptide ligands open the gate of the HLA class I binding groove. Elife 5, 1-19 (2016).
66. Liu. J. et al. Cross-Allele Cytotoxic T Lymphocyte Responses against 2009 Pandemic H1N1 Influenza A Virus among HLA-A24 and HLA-A3 Supertype-Positive Individuals. J. Virol. 86, 13281-13294 (2012).
67. Wynn, K. K. et al. Impact of clonal competition for peptide-MHC complexes on the CD8+ T-cell repertoire selection in a persistent viral infection. Blood 111, 4283-4292 (2008).
68. Kuhlman, B. et al. Design of a Novel Globular Protein Fold with Atomic-Level Accuracy. Science (80-.). 302, 1364-1369 (2003).
69. Alford, R. F. et al. The Rosetta All-Atom Energy Function for Macromolecular Modeling and Design. J. Chem. Theory Comput. 13, 3031-3048 (2017).
70. Alam. N. et al. High-resolution global peptide-protein docking using fragments-based PIPER-FlexPepDock. PLoS Comput. Biol. (2017) doi:10.1021/cm0020051.
71. Li, K., Vaudel. M., Zhang. B., Ren, Y. & Wen, B. PDV: an integrative proteomics data viewer. Bioinformatics 35, 1249-1251 (2019).
72. Kim. M., Zhong, J. & Pandey, A. Common errors in mass spectrometry-based analysis of posttranslational modifications. 16, 700-714 (2017).
73. Li, Y. et al. Mass spectrometry-based detection of protein acetylation Yu. 1077, 81-104 (2013).
74. Verrastro. I., Pasha. S., Jensen, K. T., Pitt, A. R. & Spickett, C. M. Mass spectrometry-based methods for identifying oxidized proteins in disease: Advances and challenges. Biomolecules 5, 378-411 (2015).

LENGTHY TABLES
The patent application contains a lengthy table section. A copy of the table is available in electronic form from the USPTO web site (https://seqdata.uspto.gov/?pageRequest=docDetail&DocID=US20240029819A1). An electronic copy of the table will also be available from the USPTO upon request and payment of the fee set forth in 37 CFR 1.19(b)(3).

What is claimed is:

1. A computer implemented method for generating a dataset of post translations modifications (PTM) on major histocompatibility complex (MHC) bound peptides, comprising:
receiving a mass spectrometry (MS) dataset obtained from a sample of cells associated with a target disease for treatment, the MS dataset storing a plurality of spectra data elements outputted by a MS device analyzing MHC bound peptides to generate a plurality of amino acid sequences, each spectra data element for a respective amino acid sequence of the MHC bound peptides;
receiving a reference sequence dataset storing amino acid sequences of proteins;
receiving a variable modification dataset storing a plurality of modifications each including a respective amino acid and expected mast shift;
generating a plurality of combination, each combination including a respective amino acid sequence selected from the reference sequence dataset and at least one modification selected from the variable modification dataset;
searching using a plurality of processors connected in parallel, wherein each processor searches for a respective spectra element on the plurality of combinations to identify a plurality of best peptide to spectra matches (PSMs), wherein each respective processor assigns a ranking score to respective PSM according to the respective search performed by the respective processor;
aggregating the plurality of PSMs from the plurality of processors connected in parallel to generate a main PSM list with main ranking score by computing the main ranking score from the ranking score of each respective PSM of each respective search;
selecting highest ranking PSMs according to respective main ranking scores;
storing in a modified sequence dataset, a plurality of modified sequences each including the PTM and sequences corresponding to the selected highest ranking PSMs, wherein the modified sequence dataset stores an indication of binding motifs defined by a plurality of identified PTM and corresponding sequence; and
providing the modified sequence dataset for selecting a certain binding motif having a certain PTM and corresponding amino acid sequence from the modified sequence dataset capable of specifically binding an MHC presented peptide for treatment of the target disease.

2. The method of claim 1, further comprising:
creating a training dataset by labelling each modified sequence for each respective motif of the modified sequence dataset, each modified sequence including an amino acid sequence, PTM type, and position of the PTM on the amino acid sequence, each label including an indication of one or more of: an MHC type, parent gene, and position of the motif within a full protein length; and
training a machine learning (ML) model using the training dataset,
wherein for an input of a certain modified sequence defined by a combination of an amino acid sequence and at least one PTM into the ML model, an indication of whether the certain modified sequence is predicted to fit a binding motif that binds to a cell of the MHC type is obtained as an outcome of the ML model, and
for an input of an amino acid sequence of a full protein length and PTMs into the ML model, at least one modified sequence predicted to fit a binding motif is obtained as an outcome of the ML model.

3. The method of claim 1, wherein at least one of:
the modified sequence dataset stores peptides selected from the group consisting of SEQ ID NO: 1-10746, 10817, 10819, 10820, 10823, 10824, 10826 and 10827,
the target disease comprises cancer, and the certain binding motif is selected for treating the cancer using immunotherapy, and
the MHC comprises HLA I.

4. The method of claim 1, wherein searching comprises:
allocating a respective subset of the plurality of combinations to a plurality of processors connected for parallel processing, each respective processors searching the respective spectra element on the respective subset to identify a respective set of PSM,
merging the respective set of PSM of each respective processor to create a PSM aggregation dataset,
wherein the highest ranking PSMs are selected from the PSM aggregation dataset.

5. The method of claim 4, wherein statistical parameters used in a subsequent false discovery rate (FDR) calculation are distorted by a plurality of searches of a same reference dataset over different software instances executed by the plurality of processors, and wherein merging further comprises:
removing duplicated PSM from the PSM aggregation dataset by using unmodified hits combined histogram to evaluate a number of duplicated PSM and identify the duplicated PSM for removal thereof, and
recalculating an expectation based on a restored score histogram for each PSM.

6. The method of claim 4, further comprising:
computing a plurality of quality assignment measures, and performing the following using the quality assignment measures:
validating the PTM of each member of the PSM aggregation dataset according to the quality measures;
filtering ambiguous assignments and isobaric decoys of the PSM aggregation dataset according to a filtering threshold;
ranking members of the PSM aggregation dataset; and
selecting the highest ranking PSMs according to the highest ranked member of the PSM aggregation dataset.

7. The method of claim 4, further comprising:
computing a probability score indicative of match accuracy for each PSM, wherein the highest ranking PSMs are selected according to highest probability.

8. The method of claim 1, further comprising:
dividing the PSM aggregation dataset into groups including: unmodified, standard search modification types, and other modification types, using a threshold cutoff based on respective abundance in the PSM aggregation dataset;
for each group the PSM are sorted by probability score and a threshold is set for assuring false identification is below the FDR limits.

9. The method of claim 8, when a difference in probability scores is below a defined percentage of the average probability score, the lower-ranked PSM are obtained and added to the modified sequence dataset.

10. The method of claim 8, wherein a certain PSM is identified as the highest ranking PSMs when the certain PSM is identified as having a highest probability score in one respective set of PSM and a lower ranked probability score in another respective set of PSM.

11. The method of claim 1, further comprising:
extracting the peaks from the PSM;
for each peak, computing a plurality of theoretical fragment ions for an unmodified version of the respective peptide and adjust each theoretical fragment ion according to the modification mass shift, and annotating the respective peak with the theoretical fragment ions.

12. The method of claim 11, wherein the plurality of theoretical fragment ions includes a, b, y precursor and diagnostic ions with potential ammonium and water lost in expected peptide charges.

13. The method of claim 12, further comprising:
for each PSM, searching for modification reporter ions, providing a number of b and y ions, and computing a proportion of ion current (PIC),
wherein unassigned peaks with significant intensity indicate a discrepancy between an observed spectrum defined by the respective spectra element of the plurality of PSMs and a matched peptide of the PSM.

14. The method of claim 11, further comprising:
for each PTM of each PSM, creating a window of potential site positions based on the annotated peaks, wherein at least one of: (i) including alternative site positions within the window, and (ii) including alternative combinations of modifications with equivalent mass.

15. The method of claim 1, wherein for each respective PTM of each identified PSM:
searching for identical masses or combination of masses that match the respective PTM mass shift indicative of mass decoy and/or isobaric masses, and in response to finding the identical masses or combination of masses, removing the ambiguous respective identified PSM corresponding to the respective PTM.

16. The method of claim 1, further comprising excluding PSM with total peptide mass greater than average mass of a maximum peptide length plus a tolerance value.

17. The method of claim 1, further comprising, for each respective PSM, searching in a dataset of known PSM of healthy cells and cells with the target disease for a match, and increasing likelihood of the respective PSM being included in the modified sequence dataset when the PSM is found in the dataset of known PSM.

18. A method for creating a ML model for predicting when a modified sequence binds to MHC, comprising:
creating a training dataset by labelling each modified sequence for each respective motif of the modified sequence dataset, each modified sequence including an amino acid sequence, PTM type, and position of the PTM on the amino acid sequence, the modified sequence dataset created as in claim 1, each label including an indication of one or more of: an MHC type, parent gene, and position of the motif within a full protein length; and
training a machine learning (ML) model using the training dataset,
wherein for an input of a certain modified sequence defined by a combination of an amino acid sequence and at least one PTM into the ML model, an indication of whether the certain modified sequence is predicted to fit a binding motif that binds to a cell of the MHC type is obtained as an outcome of the ML model, and
for an input of an amino acid sequence of a full protein length and PTMs into the ML model, at least one modified sequence predicted to fit a binding motif is obtained as an outcome of the ML model.

19. A computer implemented method of predicting a motif on a target HLA complex, comprising
receiving an input of one of: (i) a certain modified sequence defined by an amino acid sequence and a PTM, and (ii) an amino acid sequence of a full protein length and PTMs;
feeding the input into an ML model created as in claim 1; and
obtaining as an outcome of the ML model, for the input of (i) an indication of whether the certain modified sequence is predicted to fit a motif that binds to a cell of the MHC type, and for the input of (ii) obtaining at least one motif predicted to be created from the full protein length and PTMs.
